(19) United States
(12) Patent Application Publication
(10) Pub. No.: US 2007/0191333 A1
(43) Pub. Date: Aug. 16, 2007

(54) Antibacterial agents

(75) Inventors: Kim Marie Hutchings, Ann Arbor, MI (US); Jackie Diane Kendall, Newmarket (NZ); Sean Timothy Murphy, Ypsilanti, MI (US); Tuan Phong Tran, Canton, MI (US); Edmund Lee Ellsworth, Pinckney, MI (US); Denton Wade Hoyer, Dexter, MI (US); Jeremy Tyson Starr, Ann Arbor, MI (US)

Correspondence Address: PFIZER INC., PATENT DEPARTMENT, MS8260-1611, EASTERN POINT ROAD, GROTON, CT, 06340, US

(73) Assignee: Warner Lambert Company LLC

(21) Appl. No.: 10/580,088

(22) PCT Filed: Nov. 18, 2004
(86) PCT No.: PCT/IB04/03645

§ 371 Date: Feb. 27, 2007

Related U.S. Application Data
(60) Provisional application No. 60/523,072, filed on Nov. 18, 2003.
(60) Provisional application No. 60/606,442, filed on Sep. 2, 2004.

(51) Int. Cl. A61K 31/675; A61K 31/495; A61K 31/517; C07F 9/6512; C07D 239/88

(52) U.S. Cl. 514/210.05; 544/253; 514/81; 544/244; 514/266.3; 544/286

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

### FIELD OF THE INVENTION

The invention relates to compounds bearing a aminoquinazolinedione core structure which exhibit antibacterial activity, methods for their preparation, as well as pharmaceutically acceptable compositions comprising such compounds.

### BACKGROUND OF THE INVENTION

Antibacterial resistance is a global clinical and public health problem that has emerged with alarming rapidity in recent years. Resistance is a problem in the community as well as in health care settings, where transmission of bacteria is greatly amplified. Because multiple drug resistance is a growing problem, physicians are now confronted with infections for which there is no effective therapy. The morbidity, mortality, and financial costs of such infections pose an increasing burden for health care systems worldwide. As a result, alternative and improved agents are needed for the treatment of bacterial infections, particularly for the treatment of infections caused by resistant strains of bacteria.

### SUMMARY OF THE INVENTION

These and other needs are met by the present invention, which is directed to a compound of formula I: 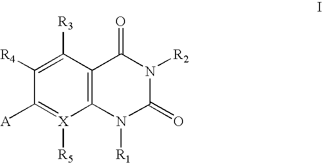
or a pharmaceutically acceptable salt thereof, wherein:
X is N or C, provided that when X is N, R₅ is absent at that position;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
   CH₂(C₃-C₆)cycloalkyl;
R₂ is H,
   NH₂, 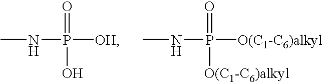
   NH(C₁-C₆)alkyl,
   NH(C₃-C₆)cycloalkyl,
   NH-aryl,
   NH-heteroaryl,
   NHSO₂—(C₁-C₆)alkyl,
   NHSO₂-aryl,
   NHSO₂-heteroaryl, 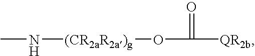
   wherein g is an integer from 1 to 10, Q is O, NH, or is absent, and R₂ₐ and R_{2A ′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 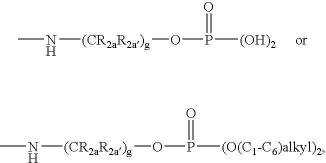
   wherein R₂ₐ and R_{2a′}are as defined above, 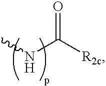
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 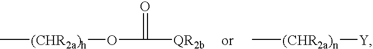
         wherein R₂ₐ, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 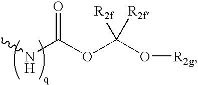
         wherein q is 0 or 1, R_{2f} and R_{2f} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy
   nitrile; or
R₁ and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
A is 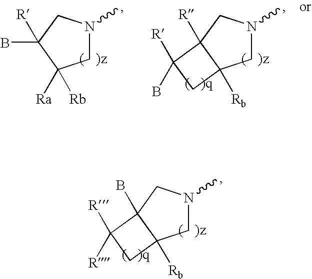
wherein z is 0, 1, or 2 and q is 0, 1, 2, or 3;
Rₐ and R_{b} are each independently H, (C₁-C₆)alkyl, (C₁-C₆)alkoxy, halo(C₁-C₆)alkyl, halo, or Rₐ and R_{b} taken together with the carbon to which they are attached form C═O, C═NO(C₁-C₆)alkyl, or a 3, 4, 5 or 6-membered substituted or unsubstituted ring;
   R′, R″, R′″, and R″″ are each independently H,
      (C₁-C₆)alkyl,
      —O(C₁-C₆)alkyl,
      halo(C₁-C₆)alkyl,
      aryl, or
      heteroaryl;
and B is 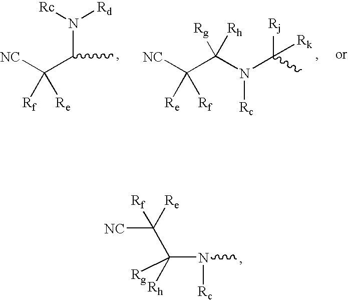
provided that when B is 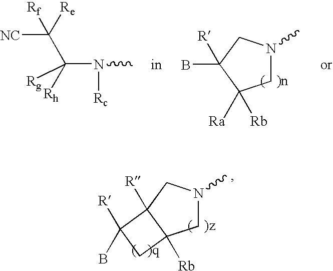
R′ is not —O(C₁-C₆)alkyl, and wherein [symbol] indicates the point of attachment;
R_{c} and R_{d} are each independently H,
   (C₁-C₆)alkylnitrile; 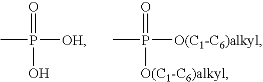
   (C₁-C₆)alkyl,
   (C₃-C₆)cycloalkyl,
   heteroaryl,
   SO₂—(C₁-C₆)alkyl,
   SO₂-aryl,
   SO₂-heteroaryl, 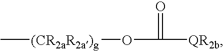
   wherein g is an integer from 1 to 10, Q is as defined above, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 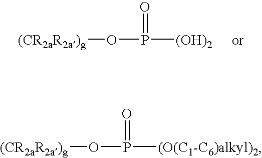
   wherein R₂ₐ and R_{2a′} are as defined above, 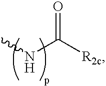
   wherein [symbol] indicates the point of attachment, p is 0 or 1, and
      R₂ is H,
         (C₁-C₆)alkyl,
         O(C₁-C₆)alkyl,
         (C₃-C₇)cycloalkyl,
         aryl,
         heterocyclo,
         heteroaryl, or 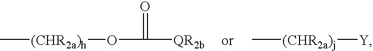
         wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 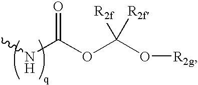
         wherein q is 0 or 1, R_{2f} and R_{2f} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
         heteroaryl;
Rₑ and R_{f} are each independently H, C₁-C₆ alkyl, haloalkyl, halo, or Rₑ and R_{f} taken together with the carbon to which they are attached form a 3, 4, 5 or 6-membered substituted or unsubstituted ring;
R_{g} and Rₕ are each independently H, C₁-C₆ alkyl, haloalkyl, or taken together with the carbon to which they are attached to form a 3, 4, 5 or 6-membered substituted or unsubstituted ring; and
Rⱼ and Rₖ are each independently H, (C₁-C₆)alkyl, haloalkyl, (C₁-C₆)alkyl-NR_{c}R_{d}, (C₁-C₆)alkyl-OR_{c}, aryl, heteroaryl, heterocycle, 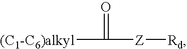
wherein Z is O or NR_{c}, or Rⱼ and Rₖ taken together with the carbon to which they are attached to form a 3, 4, 5 or 6-membered substituted or unsubstituted ring.

What is also provided is a compound of formula II: 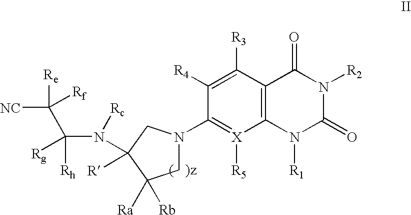
or a pharmaceutically acceptable salt thereof, wherein:
X is N or C, provided that when X is N, R₅ is absent at that position;
n is 0, 1, or 2;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
   CH₂(C₃-C₆)cycloalkyl;
R₂ is H,
   NH₂, 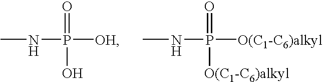
   NH(C₁-C₆)alkyl,
   NH(C₃-C₆)cycloalkyl,
   NH-aryl,
   NH-heteroaryl,
   NHSO₂—(C₁-C₆)alkyl,
   NHSO₂-aryl,
   NHSO₂-heteroaryl, 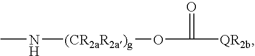
   wherein g is an integer from 1 to 10, Q is O, NH, or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 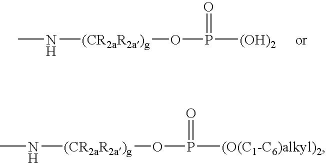
   wherein R₂ₐ and R_{2a′} are as defined above, 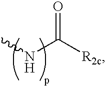
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 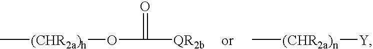
         wherein R₂ₐ, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 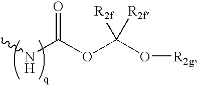
         wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy
   nitrile; or
R₁ and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
z is 0, 1, or 2;
R′ is H,
   (C₁-C₆)alkyl,
   halo(C₁-C₆)alkyl,
   aryl, or
   heteroaryl;
Rₐ and R_{b} are each independently H, (C₁-C₆)alkyl, (C₁-C₆)alkoxy, halo(C₁-C₆)alkyl, halo, or Rₐ and R_{b} taken together with the carbon to which they are attached form C═O, C═NO(C₁-C₆)alkyl, or a 3, 4, 5 or 6-membered substituted or unsubstituted ring;
R_{c} is H,
   (C₁-C₆)alkylnitrile; 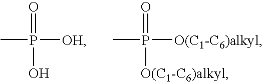
   (C₁-C₆)alkyl,
   (C₃-C₆)cycloalkyl,
   heteroaryl,
   SO₂—(C₁-C₆)alkyl,
   SO₂-aryl,
   SO₂-heteroaryl, 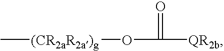
   wherein g is an integer from 1 to 10, Q is as defined above, and R₂ₐ and R_{2a′}, are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 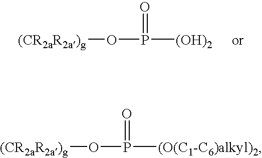
   wherein R₂ₐ and R_{2a′} are as defined above, 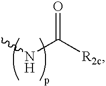
   wherein [symbol] indicates the point of attachment, p is 0 or 1, and
      R₂ is H,
         (C₁-C₆)alkyl,
         O(C₁-C₆)alkyl,
         (C₃-C₇)cycloalkyl,
         aryl,
         heterocyclo,
         heteroaryl, or 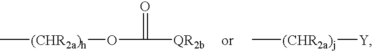
         wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 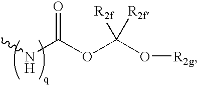
         wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
         heteroaryl;
Rₑ and R_{f} are each independently H, C₁-C₆ alkyl, haloalkyl, halo, or Rₑ and R_{f} taken together with the carbon to which they are attached form a 3, 4, 5 or 6-membered substituted or unsubstituted ring; and
R_{g} and Rₕ are each independently H, C₁-C₆ alkyl, haloalkyl, or taken together with the carbon to which they are attached to form a 3, 4, 5 or 6-membered substituted or unsubstituted ring.

What is also provided is a compound which is 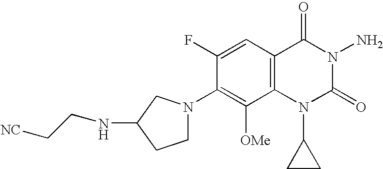
3-[1-(3-Amino-1-cyclopropyl-6-fluoro-8-methoxy-2,4-dioxo-1,2,3,4-tetrahydro-quinazolin-7-yl)-pyrrolidin-3-ylamino]-propionitrile; 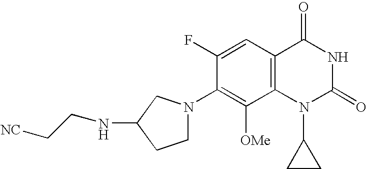
3-[1-(1-Cyclopropyl-6-fluoro-8-methoxy-2,4-dioxo-1,2,3,4-tetrahydro-quinazolin-7-yl)-pyrrolidin-3-ylamino]-propionitrile; 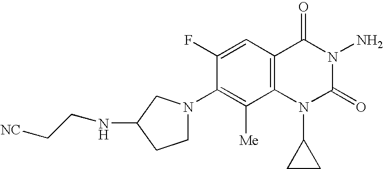
3-[1-(3-Amino-1-cyclopropyl-6-fluoro-8-methyl-2,4-dioxo-1,2,3,4-tetrahydro-quinazolin-7-yl)-pyrrolidin-3-ylamino]-propionitrile; 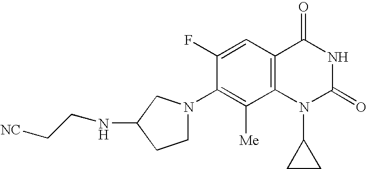
3-[1-(1-Cyclopropyl-6-fluoro-8-methyl-2,4-dioxo-1,2,3,4-tetrahydro-quinazolin-7-yl)-pyrrolidin-3-ylamino]-propionitrile; 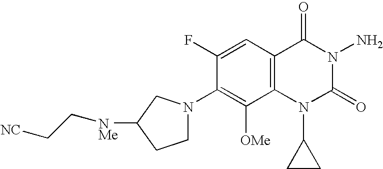
3-{[1-(3-Amino-1-cyclopropyl-6-fluoro-8-methoxy-2,4-dioxo-1,2,3,4-tetrahydro-quinazolin-7-yl)-pyrrolidin-3-yl]-methyl-amino}-propionitrile; 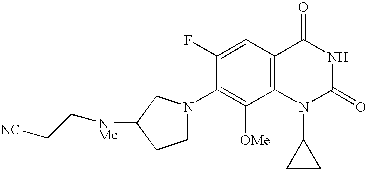
3-{[1-(1-Cyclopropyl-6-fluoro-8-methoxy-2,4-dioxo-1,2,3,4-tetrahydro-quinazolin-7-yl)-pyrrolidin-3-yl]-methyl-amino}-propionitrile; 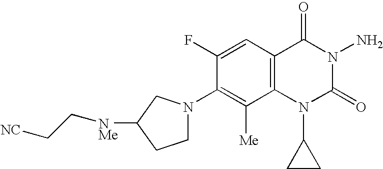
3-{[1-(3-Amino-1-cyclopropyl-6-fluoro-8-methyl-2,4-dioxo-1,2,3,4-tetrahydro-quinazolin-7-yl)-pyrrolidin-3-yl]-methyl-amino}-propionitrile; or 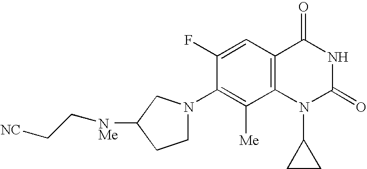
3-{[1-(1-Cyclopropyl-6-fluoro-8-methyl-2,4-dioxo-1,2,3,4-tetrahydro-quinazolin-7-yl)-pyrrolidin-3-yl]-methyl-amino}-propionitrile.

What is also provided is a compound of formula III: 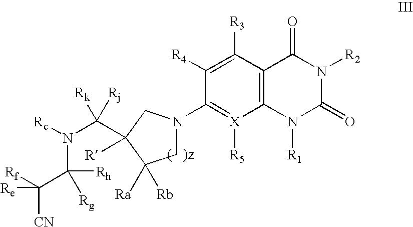
or a pharmaceutically acceptable salt thereof, wherein:
X is N or C, provided that when X is N, R₅ is absent at that position;
n is 0, 1, or 2;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
   CH₂(C₃-C₆)Cycloalkyl;
R₂ is H,
   NH₂, 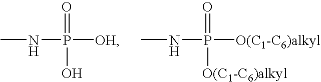
   NH(C₁-C₆)alkyl,
   NH(C₃-C₆)cycloalkyl,
   NH-aryl,
   NH-heteroaryl,
   NHSO₂—(C₁-C₆)alkyl,
   NHSO₂-aryl,
   NHSO₂-heteroaryl, 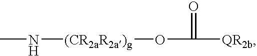
   wherein g is an integer from 1 to 10, Q is O, NH, or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 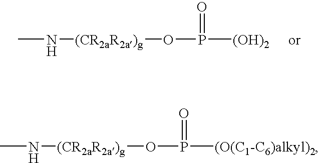
   wherein R₂ₐ and R_{2a′} are as defined above, 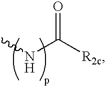
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 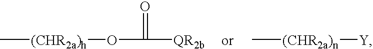
         wherein R₂, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 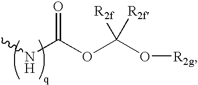
         wherein q is 0 or 1, R_{2f ′} and R_{2f} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy
   nitrile; or
R₁ and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
z is 0, 1, or 2;
R′ is H,
   (C₁-C₆)alkyl,
   —O(C₁-C₆)alkyl,
   halo(C₁-C₆)alkyl,
   aryl, or
   heteroaryl;
Rₐ and R_{b} are each independently H, (C₁-C₆)alkyl, (C₁-C₆)alkoxy, halo(C₁-C₆)alkyl, halo, or Rₐ and R_{b} taken together with the carbon to which they are attached form C═O, C═NO(C₁-C₆)alkyl, or a 3, 4, 5 or 6-membered substituted or unsubstituted ring;
R_{c} is H,
   (C₁-C₆)alkylnitrile; 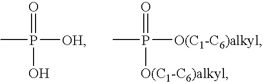
   (C₁-C₆)alkyl,
   (C₃-C₆)cycloalkyl,
   heteroaryl,
   SO₂—(C₁-C₆)alkyl,
   SO₂-aryl,
   SO₂-heteroaryl, 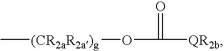
   wherein g is an integer from 1 to 10, Q is as defined above, and R₂ₐ and R₂ₐ are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 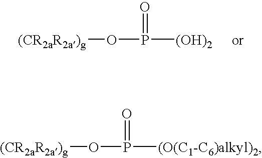
   wherein R₂ₐ and R_{2a′} are as defined above, 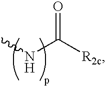
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 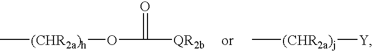
         wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 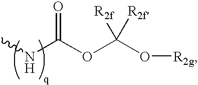
         wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
         heteroaryl;
Rₑ and R_{f} are each independently H, C₁-C₆ alkyl, haloalkyl, halo, or Rₑ and R_{f} taken together with the carbon to which they are attached form a 3, 4, 5 or 6-membered substituted or unsubstituted ring;
R_{g} and Rₕ are each independently H, C₁-C₆ alkyl, haloalkyl, or taken together with the carbon to which they are attached to form a 3, 4, 5 or 6-membered substituted or unsubstituted ring; and
Rⱼ and Rₖ are each independently H, (C₁-C₆)alkyl, haloalkyl, (C₁-C₆)alkyl-NR_{c}R_{d}, (C₁-C₆)alkyl-OR_{c}, aryl, heteroaryl, heterocycle, 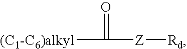
wherein Z is O or NR_{c}, or Rⱼ and Rₖ taken together with the carbon to which they are attached to form a 3, 4, 5 or 6-membered substituted or unsubstituted ring.

What is also provided is a compound which is 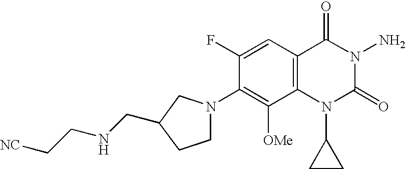
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-amino}-propionitrile; 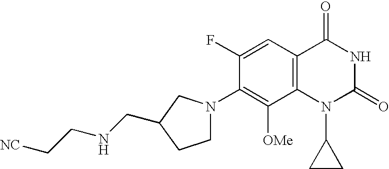
3-{[1-(1-Cyclopropyl-6-fluoro-8-methoxy-2,4-dioxo-1,2,3,4-tetrahydro-quinazolin-7-yl)-pyrrolidin-3-ylmethyl]-amino}-propionitrile; 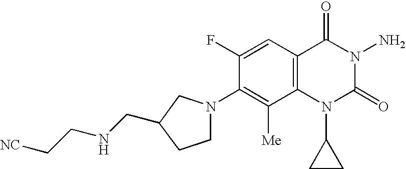
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-amino}-propionitrile; 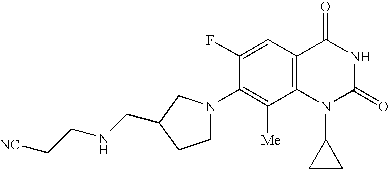
3-{[1-(1-Cyclopropyl-6-fluoro-8-methyl-2,4-dioxo-1,2,3,4-tetrahydro-quinazolin-7-yl)-pyrrolidin-3-ylmethyl]-amino}-propionitrile; 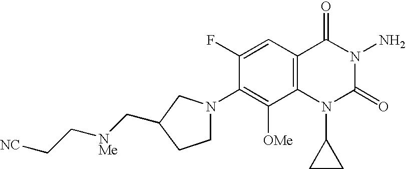
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-methyl-amino}-propionitrile; 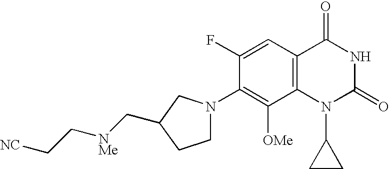
3-{[1-(1-Cyclopropyl-6-fluoro-8-methoxy-2,4-dioxo-1,2,3,4-tetrahydro-quinazolin-7-yl)-pyrrolidin-3-ylmethyl]-methyl-amino}-propionitrile; 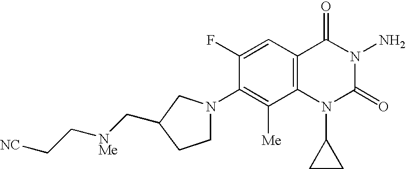
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-propionitrile; 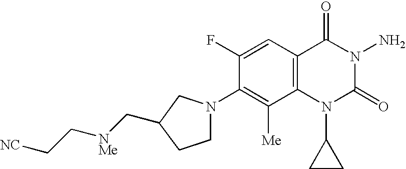
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-propionitrile; 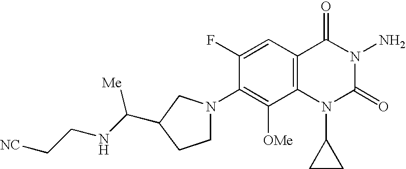
3-{1-[1-(3-Amino-1-cyclopropyl-6-fluoro-8-methoxy-2,4-dioxo-1,2,3,4-tetrahydro-quinazolin-7-yl)-pyrrolidin-3-yl]-ethylamino}-propionitrile; 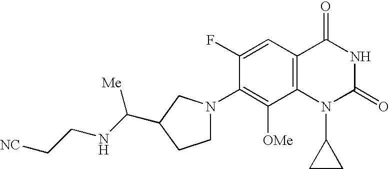
3-{1-[1-(1-Cyclopropyl-6-fluoro-8-methoxy-2,4-dioxo-1,2,3,4-tetrahydro-quinazolin-7-yl)-pyrrolidin-3-yl]-ethylamino}-propionitrile; 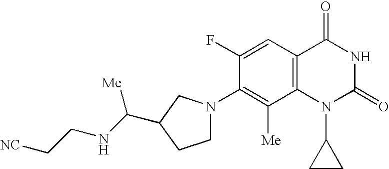
3-{1-[1-(1-Cyclopropyl-6-fluoro-8-methyl-2,4-dioxo-1,2,3,4-tetrahydro-quinazolin-7-yl)-pyrrolidin-3-yl]-ethylamino}-propionitrile; 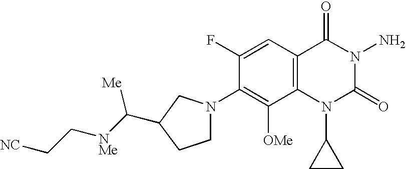
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-ethyl}-methyl-amino)-propionitrile; 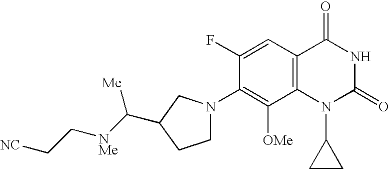
3-({1-[1-(1-Cyclopropyl-6-fluoro-8-methoxy-2,4-dioxo-1,2,3,4-tetrahydro-quinazolin-7-yl)-pyrrolidin-3-yl]-ethyl}-methyl-amino)-propionitrile; 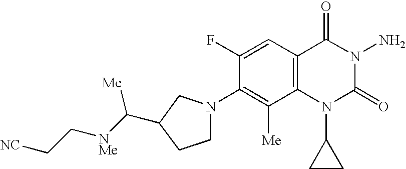
3-({1-[1-(3-Amino-1-cyclopropyl-6-fluoro-8-methyl-2,4-dioxo-1,2,3,4-tetrahydro-quinazolin-7-yl)-pyrrolidin-3-yl]-ethyl}-methyl-amino)-propionitrile; 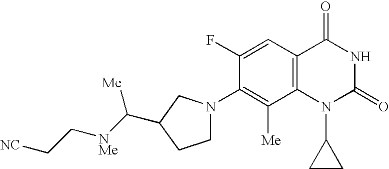
3-({1-[1-(1-Cyclopropyl-6-fluoro-8-methyl-2,4-dioxo-1,2,3,4-tetrahydro-quinazolin-7-yl)-pyrrolidin-3-yl]-ethyl}-methyl-amino)-propionitrile; 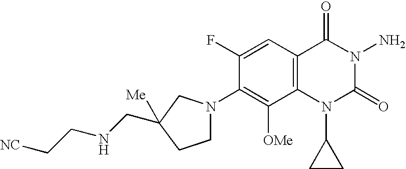
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-amino}-propionitrile; 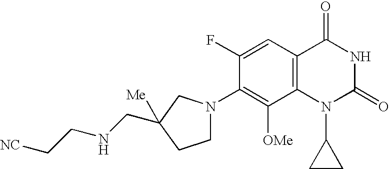
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-amino}-propionitrile; 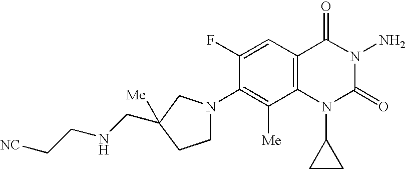
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-amino}-propionitrile; 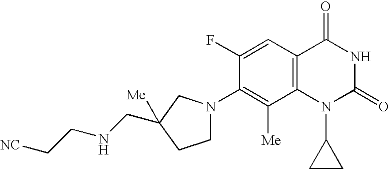
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-amino}-propionitrile; 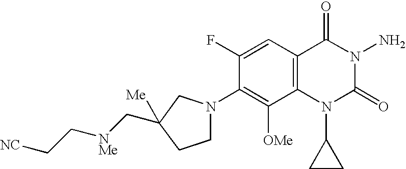
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-methyl-amino}-propionitrile; 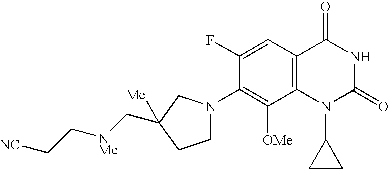
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-methyl-amino}-propionitrile; 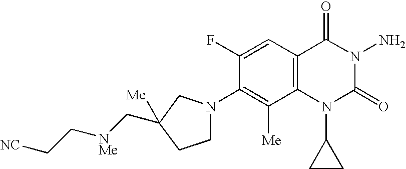
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-methyl-amino}-propionitrile; 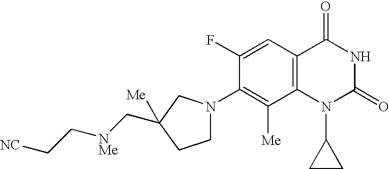
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-methyl-amino}-propionitrile; 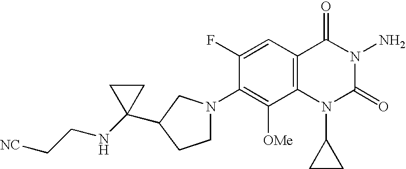
3-{1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropylamino}-propionitrile; 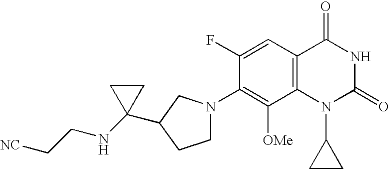
3-{1-[1-(1-Cyclopropyl-6-fluoro-8-methoxy-2,4-dioxo-1,2,3,4-tetrahydro-quinazolin-7-yl)-pyrrolidin-3-yl]-cyclopropylamino}-propionitrile; 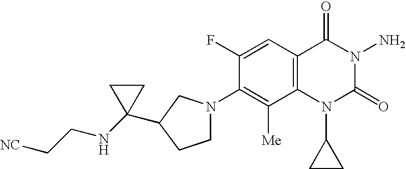
3-{1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropylamino}-propionitrile; 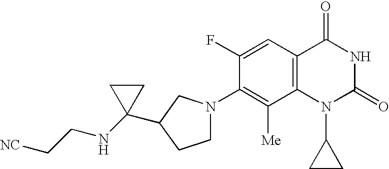
3-{1-[1-(1-Cyclopropyl-6-fluoro-8-methyl-2,4-dioxo-1,2,3,4-tetrahydro-quinazolin-7-yl)-pyrrolidin-3-yl]-cyclopropylamino}-propionitrile; 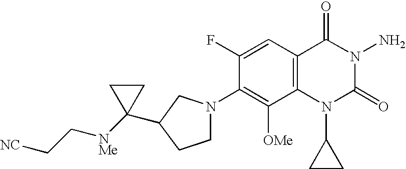
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropyl}-methyl-amino)-propionitrile; 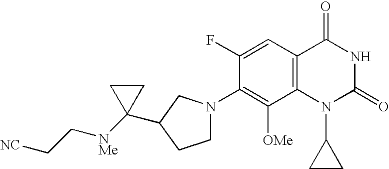
3-({1-[1-(1-Cyclopropyl-6-fluoro-8-methoxy-2,4-dioxo-1,2,3,4-tetrahydro-quinazolin-7-yl)-pyrrolidin-3-yl]-cyclopropyl}-methyl-amino)-propionitrile; 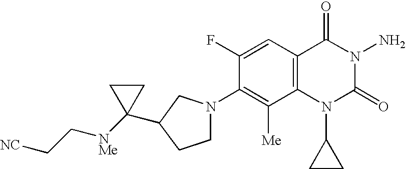
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-methyl-amino)-propionitrile; 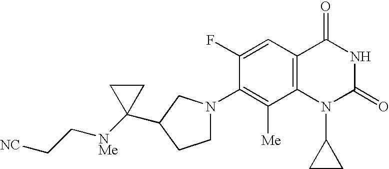
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-methyl-amino)-propionitrile; 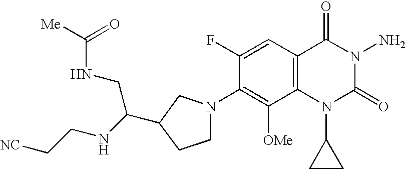
N-[2-[1-(3-Amino-1-cyclopropyl-6-fluoro-8-methoxy-2,4-dioxo-1,2,3,4-tetrahydro-quinazolin-7-yl)-pyrrolidin-3-yl]-2-(2-cyano-ethylamino)-ethyl]-acetamide; 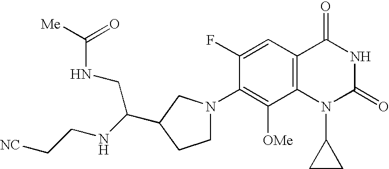
N-[2-[1-(1-Cyclopropyl-6-fluoro-8-methoxy-2,4-dioxo-1,2,3,4-tetrahydro-quinazolin-7-yl)-pyrrolidin-3-yl]-2-(2-cyano-ethylamino)-ethyl]-acetamide; 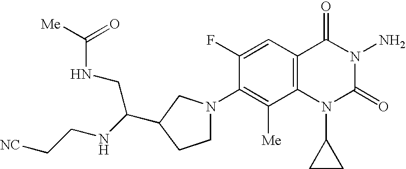
N-[2-[1-(3-Amino-1-cyclopropyl-6-fluoro-8-methyl-2,4-dioxo-1,2,3,4-tetrahydro-quinazolin-7-yl)-pyrrolidin-3-yl]-2-(2-cyano-ethylamino)-ethyl]-acetamide; 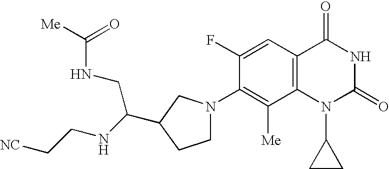
N-[2-[1-(1-Cyclopropyl-6-fluoro-8-methyl-2,4-dioxo-1,2,3,4-tetrahydro-quinazolin-7-yl)-pyrrolidin-3-yl]-2-(2-cyano-ethylamino)-ethyl]-acetamide; 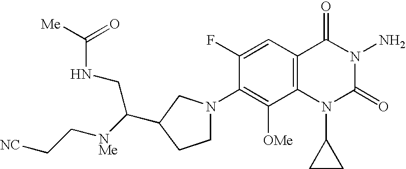
N-{2-[1-(3-Amino-1-cyclopropyl-6-fluoro-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide; 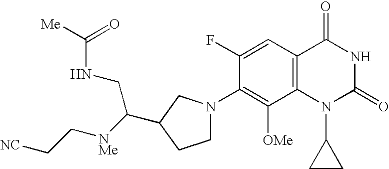
N-{2-[1-(1-Cyclopropyl-6-fluoro-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide; 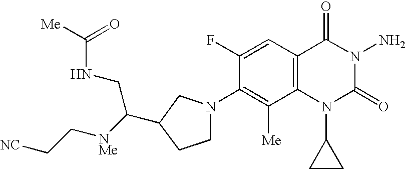
N-{2-[1-(3-Amino-1-cyclopropyl-6-fluoro-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide; 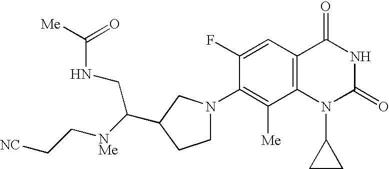
N-{2-[1-(1-Cyclopropyl-6-fluoro-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide; 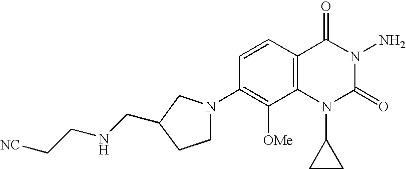
3-{[1-(3-Amino-1-cyclopropyl-8-methoxy-2,4-dioxo-1,2,3,4-tetrahydro-quinazolin-7-yl)-pyrrolidin-3-ylmethyl]-amino}-propionitrile; 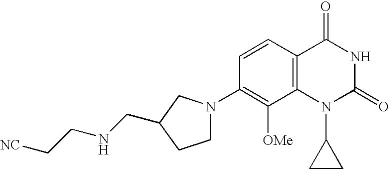
3-{[1-(1-Cyclopropyl-8-methoxy-2,4-dioxo-1,2,3,4-tetrahydro-quinazolin-7-yl)-pyrrolidin-3-ylmethyl]-amino}-propionitrile; 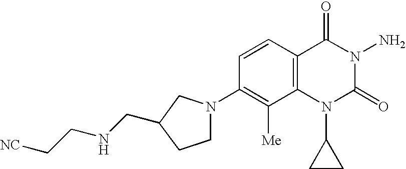
3-{[1-(3-Amino-1-cyclopropyl-8-methyl-2,4-dioxo-1,2,3,4-tetrahydro-quinazolin-7-yl)-pyrrolidin-3-ylmethyl]-amino}-propionitrile; 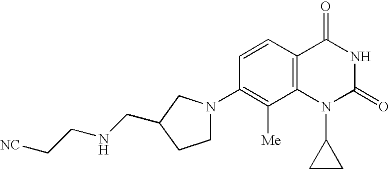
3-{[1-(1-Cyclopropyl-8-methyl-2,4-dioxo-1,2,3,4-tetrahydro-quinazolin-7-yl)-pyrrolidin-3-ylmethyl]-amino}-propionitrile; 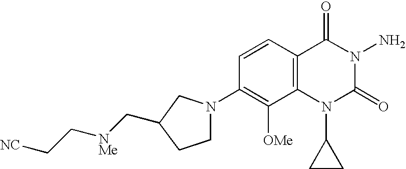
3-{[1-(3-Amino-1-cyclopropyl-8-methoxy-2,4-dioxo-1,2,3,4-tetrahydro-quinazolin-7-yl)-pyrrolidin-3-ylmethyl]-methyl-amino}-propionitrile; 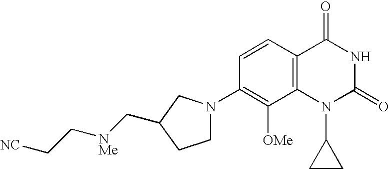
3-{[1-(1-Cyclopropyl-8-methoxy-2,4-dioxo-1,2,3,4-tetrahydro-quinazolin-7-yl)-pyrrolidin-3-ylmethyl]-methyl-amino}-propionitrile; 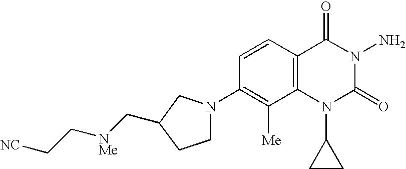
3-{[1-(3-Amino-1-cyclopropyl-8-methyl-2,4-dioxo-1,2,3,4-tetrahydro-quinazolin-7-yl)-pyrrolidin-3-ylmethyl]-methyl-amino}-propionitrile; 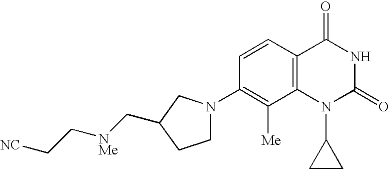
3-{[1-(1-Cyclopropyl-8-methyl-2,4-dioxo-1,2,3,4-tetrahydro-quinazolin-7-yl)-pyrrolidin-3-ylmethyl]-methyl-amino}-propionitrile; 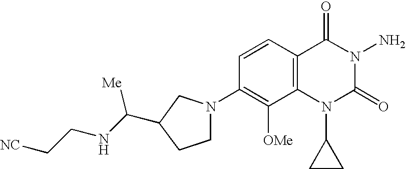
3-{1-[1-(3-Amino-1-cyclopropyl-8-methoxy-2,4-dioxo-1,2,3,4-tetrahydro-quinazolin-7-yl)-pyrrolidin-3-yl]-ethylamino}-propionitrile; 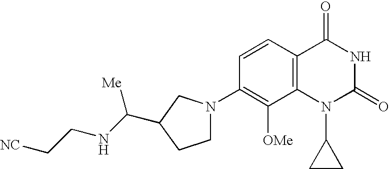
3-{1-[1-(1-(1-Cyclopropyl-8-methoxy-2,4-dioxo-1,2,3,4-tetrahydro-quinazolin-7-yl)-pyrrolidin-3-yl]-ethylamino}-propionitrile; 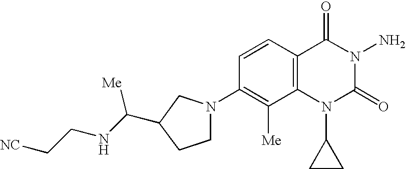
3-{1-[1-(3-Amino-1-cyclopropyl-8-methyl-2,4-dioxo-1,2,3,4-tetrahydro-quinazolin-7-yl)-pyrrolidin-3-yl]-ethylamino}-propionitrile; 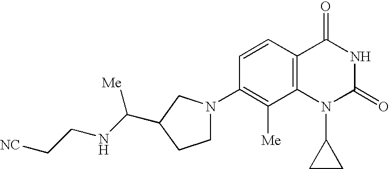
3-{1-[1-(1-Cyclopropyl-8-methyl-2,4-dioxo-1,2,3,4-tetrahydro-quinazolin-7-yl)-pyrrolidin-3-yl]-ethylamino}-propionitrile; 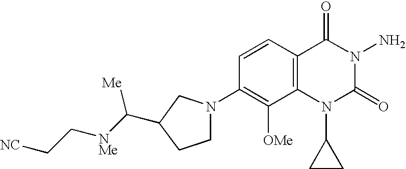
3-({1-[1-(3-Amino-1-cyclopropyl-8-methoxy-2,4-dioxo-1,2,3,4-tetrahydro-quinazolin-7-yl)-pyrrolidin-3-yl]-ethyl}-methyl-amino)-propionitrile; 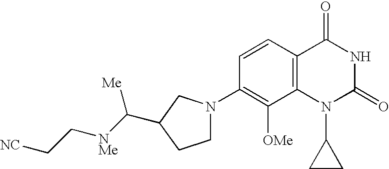
3-({1-[1-(1-Cyclopropyl-8-methoxy-2,4-dioxo-1,2,3,4-tetrahydro-quinazolin-7-yl)-pyrrolidin-3-yl]-ethyl}-methyl-amino)-propionitrile; 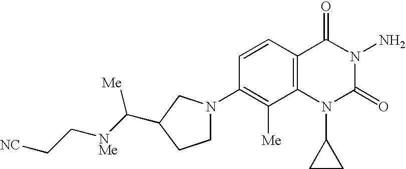
3-({1-[1-(3-Amino-1-cyclopropyl-8-methyl-2,4-dioxo-1,2,3,4-tetrahydro-quinazolin-7-yl)-pyrrolidin-3-yl]-ethyl}-methyl-amino)-propionitrile; 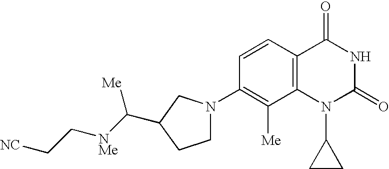
3-({1-[1-(1-Cyclopropyl-8-methyl-2,4-dioxo-1,2,3,4-tetrahydro-quinazolin-7-yl)-pyrrolidin-3-yl]-ethyl}-methyl-amino)-propionitrile; 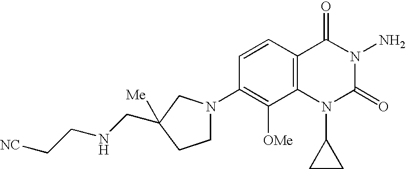
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-amino}-propionitrile; 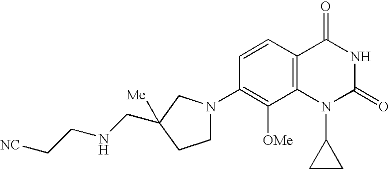
3-{[1-(3-Amino-1-cyclopropyl-8-methoxy-2,4-dioxo-1,2,3,4-tetrahydro-quinazolin-7-yl)-3-methyl-pyrrolidin-3-ylmethyl-amino}-propionitrile; 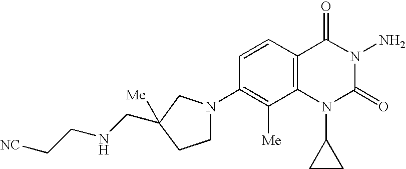
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-amino}-propionitrile; 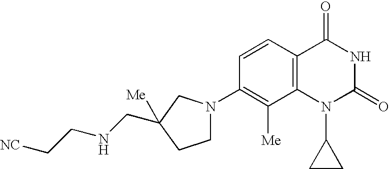
3-{[1-(1-Cyclopropyl-8-methyl-2,4-dioxo-1,2,3,4-tetrahydro-quinazolin-7-yl)-3-methyl-pyrrolidin-3-ylmethyl]-amino}-propionitrile; 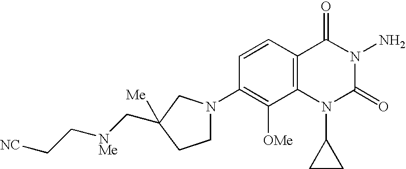
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-methyl-amino}-propionitrile; 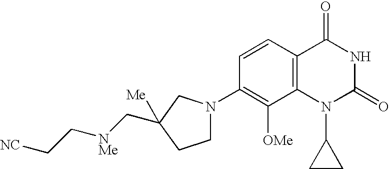
3-{[1-(1-Cyclopropyl-8-methoxy-2,4-dioxo-1,2,3,4-tetrahydro-quinazolin-7-yl)-3-methyl-pyrrolidin-3-ylmethyl]-methyl-amino}-propionitrile; 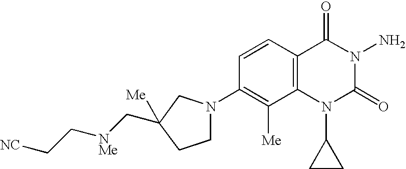
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-methyl-amino]-propionitrile; 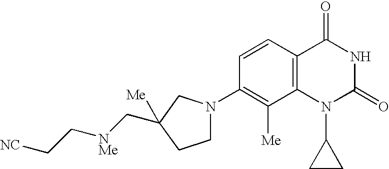
3-{[1-(1-Cyclopropyl-8-methyl-2,4-dioxo-1,2,3,4-tetrahydro-quinazolin-7-yl)-3-methyl-pyrrolidin-3-ylmethyl]-methyl-amino}-propionitrile; 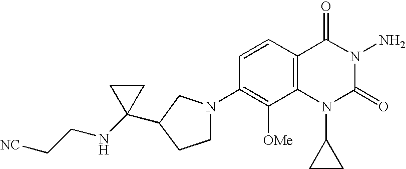
3-{1-[1-(3-Amino-1-cyclopropyl-8-methoxy-2,4-dioxo-1,2,3,4-tetrahydro-quinazolin-7-yl)-pyrrolidin-3-yl]-cyclopropylamino}-propionitrile; 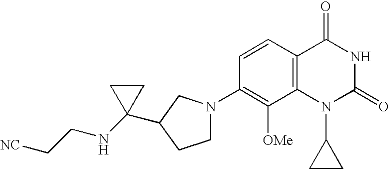
3-(1-[1-(1-Cyclopropyl-8-methoxy-2,4-dioxo-1,2,3,4-tetrahydro-quinazolin-7-yl)-pyrrolidin-3-yl]-cyclopropylamino}-propionitrile; 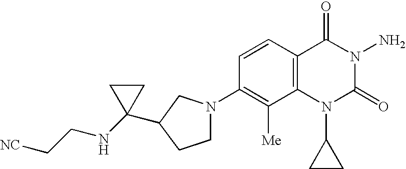
3-{1-[1-(3-Amino-1-cyclopropyl-8-methyl-2,4-dioxo-1,2,3,4-tetrahydro-quinazolin-7-yl)-pyrrolidin-3-yl]-cyclopropylamino}-propionitrile; 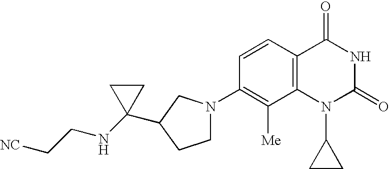
3-{1-[1-(1-Cyclopropyl-8-methyl-2,4-dioxo-1,2,3,4-tetrahydro-quinazolin-7-yl)-pyrrolidin-3-yl]-cyclopropylamino}-propionitrile; 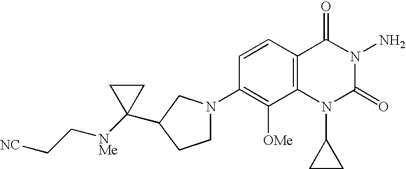
3-({1-[1-(3-Amino-1-cyclopropyl-8-methoxy-2,4-dioxo-1,2,3,4-tetrahydro-quinazolin-7-yl)-pyrrolidin-3-yl]-cyclopropyl}-methyl-amino)-propionitrile; 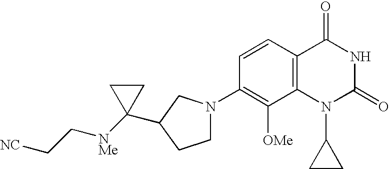
3-({1-[1-(1-cyclopropyl-8-methoxy-2,4-dioxo-1,2,3,4-tetrahydro-quinazolin-7-yl)-pyrrolidin-3-yl]-cyclopropyl}-methyl-amino)-propionitrile; 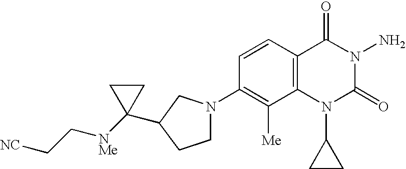
3-({1-[1-(3-Amino-1-cyclopropyl-8-methyl-2,4-dioxo-1,2,3,4-tetrahydro-quinazolin-7-yl)-pyrrolidin-3-yl]-cyclopropyl}-methyl-amino)-propionitrile; 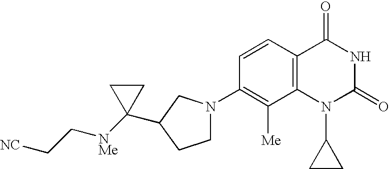
3-({1-[1-(1-Cyclopropyl-8-methyl-2,4-dioxo-1,2,3,4-tetrahydro-quinazolin-7-yl)-pyrrolidin-3-yl]-cyclopropyl}-methyl-amino)-propionitrile; 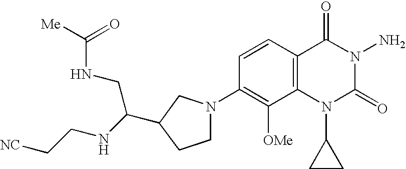
N-[2-[1-(3-Amino-1-cyclopropyl-8-methoxy-2,4-dioxo-1,2,3,4-tetrahydro-quinazolin-7-yl)-pyrrolidin-3-yl]-2-(2-cyano-ethylamino)-ethyl]-acetamide; 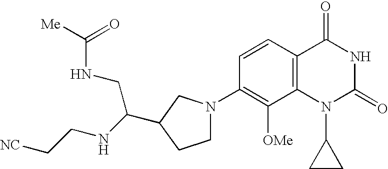
N-[2-[1-(1-Cyclopropyl-8-methoxy-2,4-dioxo-1,2,3,4-tetrahydro-quinazolin-7-yl)-pyrrolidin-3-yl]-2-(2-cyano-ethylamino)-ethyl]-acetamide; 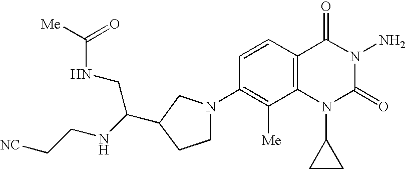
N-[2-[1-(3-Amino-1-cyclopropyl-8-methyl-2,4-dioxo-1,2,3,4-tetrahydro-quinazolin-7-yl)-pyrrolidin-3-yl]-2-(2-cyano-ethylamino)-ethyl]-acetamide; 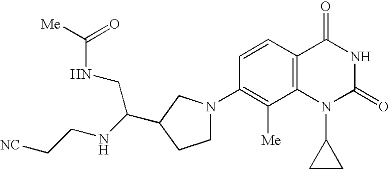
N-[2-[1-(1-Cyclopropyl-8-methyl-2,4-dioxo-1,2,3,4-tetrahydro-quinazolin-7-yl)-pyrrolidin-3-yl]-2-(2-cyano-ethylamino)-ethyl]-acetamide; 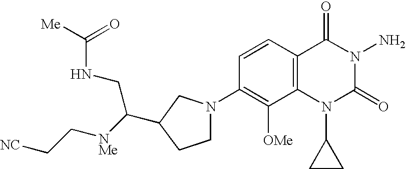
N-{2-[1-(3-Amino-1-cyclopropyl-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide; 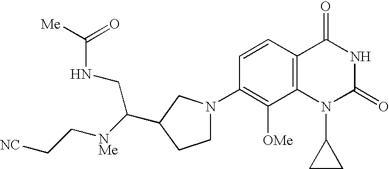
N-{2-[1-(1-Cyclopropyl-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide; 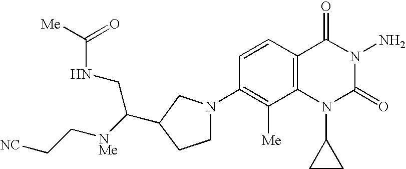
N-{2-[1-(3-Amino-1-cyclopropyl-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide; 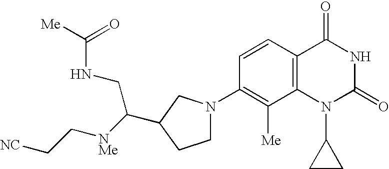
N-{2-[1-(1-Cyclopropyl-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide; 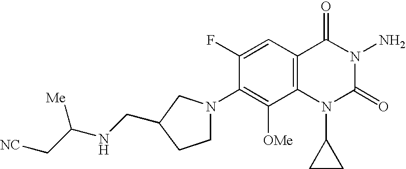
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-amino}-butyronitrile; 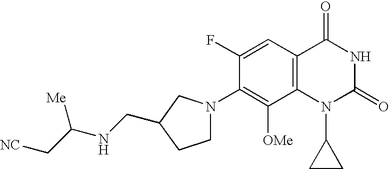
3-{[1-(1-Cyclopropyl-6-fluoro-8-methoxy-2,4-dioxo-1,2,3,4-tetrahydro-quinazolin-7-yl)-pyrrolidin-3-ylmethyl]-amino}-butyronitrile; 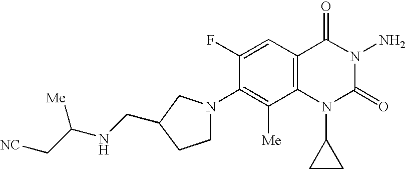
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-amino}-butyronitrile; 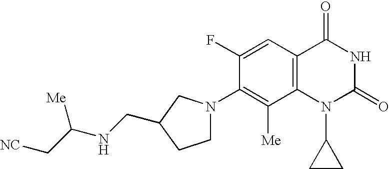
3-{[1-(1-Cyclopropyl-6-fluoro-8-methyl-2,4-dioxo-1,2,3,4-tetrahydro-quinazolin-7-yl)-pyrrolidin-3-ylmethyl]-amino}-butyronitrile; 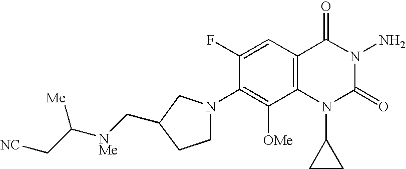
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-methyl-amino}-butyronitrile; 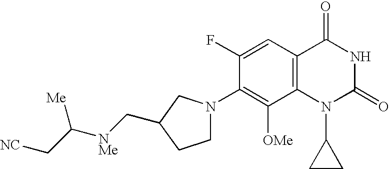
3-{[1-(1-Cyclopropyl-6-fluoro-8-methoxy-2,4-dioxo-1,2,3,4-tetrahydro-quinazolin-7-yl)-pyrrolidin-3-ylmethyl]-methyl-amino}-butyronitrile; 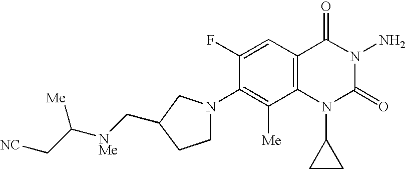
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-butyronitrile; 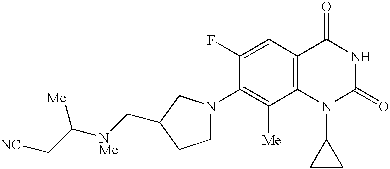
3-{[1-(1-Cyclopropyl-6-fluoro-8-methyl-2,4-dioxo-1,2,3,4-tetrahydro-quinazolin-7-yl)-pyrrolidin-3-ylmethyl]-methyl-amino}-butyronitrile; 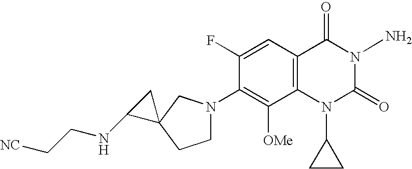
3-[5-(3-Amino-1-cyclopropyl-6-fluoro-8-methoxy-2,4-dioxo-1,2,3,4-tetrahydro-quinazolin-7-yl)-5-aza-spiro[2.4]hept-1-ylamino]-propionitrile; 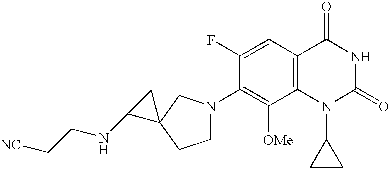
3-[5-(1-Cyclopropyl-6-fluoro-8-methoxy-2,4-dioxo-1,2,3,4-tetrahydro-quinazolin-7-yl)-5-aza-spiro[2.4]hept-1-ylamino]-propionitrile; 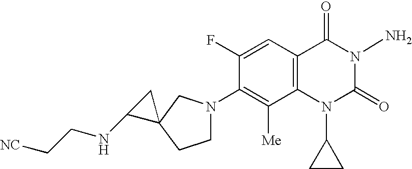
3-[5-(3-Amino-1-cyclopropyl-6-fluoro-8-methyl-2,4-dioxo-1,2,3,4-tetrahydro-quinazolin-7-yl)-5-aza-spiro[2.4]hept-1-ylamino]-propionitrile; 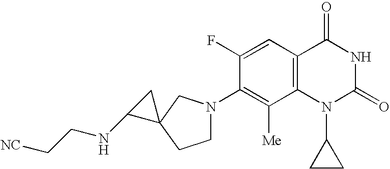
3-[5-(1-Cyclopropyl-6-fluoro-8-methyl-2,4-dioxo-1,2,3,4-tetrahydro-quinazolin-7-yl)-5-aza-spiro[2.4]hept-1-ylamino]-propionitrile; 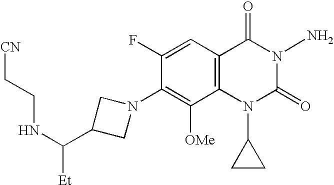
3-{1-[1-(3-Amino-1-cyclopropyl-6-fluoro-8-methoxy-2,4-dioxo-1,2,3,4-tetrahydro-quinazolin-7-yl)-azetidin-3-yl]-propylamino}-propionitrile; 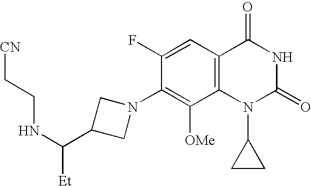
3-{1-[1-(1-Cyclopropyl-6-fluoro-8-methoxy-2,4-dioxo-1,2,3,4-tetrahydro-quinazolin-7-yl)-azetidin-3-yl]-propylamino}-propionitrile; 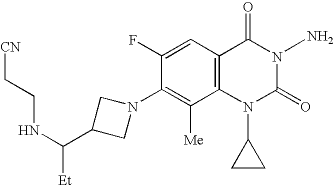
3-{1-[1-(3-Amino-1-cyclopropyl-6-fluoro-8-methyl-2,4-dioxo-1,2,3,4-tetrahydro-quinazolin-7-yl)-azetidin-3-yl]-propylamino}-propionitrile; 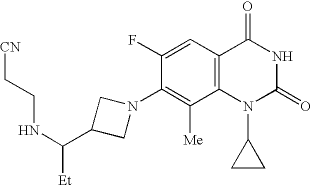
3-{1-[1-(1-Cyclopropyl-6-fluoro-8-methyl-2,4-dioxo-1,2,3,4-tetrahydro-quinazolin-7-yl)-azetidin-3-yl]-propylamino}-propionitrile; 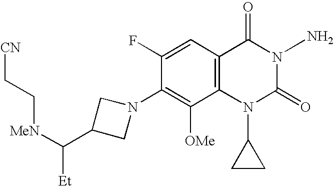
3-({1-[1-(3-Amino-1-cyclopropyl-6-fluoro-8-methoxy-2,4-dioxo-1,2,3,4-tetrahydro-quinazolin-7-yl)-azetidin-3-yl]-propyl}-methyl-amino)-propionitrile; 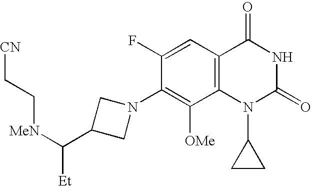
3-({1-[1-(1-Cyclopropyl-6-fluoro-8-methoxy-2,4-dioxo-1,2,3,4-tetrahydro-quinazolin-7-yl)-azetidin-3-yl]-propyl}-methyl-amino)-propionitrile; 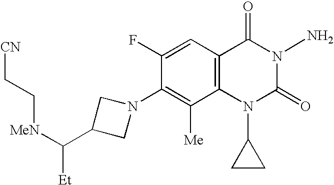
3-({1-[1-(3-Amino-1-cyclopropyl-6-fluoro-8-methyl-2,4-dioxo-1,2,3,4-tetrahydro-quinazolin-7-yl)-azetidin-3-yl]-propyl}-methyl-amino)-propionitrile; 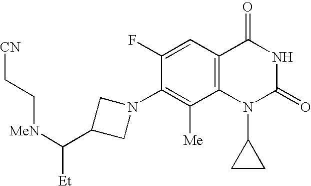
3-({1-[1-(1-Cyclopropyl-6-fluoro-8-methyl-2,4-dioxo-1,2,3,4-tetrahydro-quinazolin-7-yl)-azetidin-3-yl]-propyl}-methyl-amino)-propionitrile; 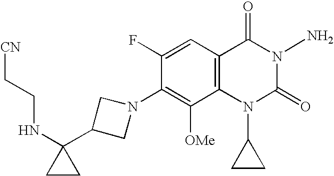
3-{1-[1-(3-Amino-1-cyclopropyl-6-fluoro-8-methoxy-2,4-dioxo-1,2,3,4-tetrahydro-quinazolin-7-yl)-azetidin-3-yl]-cyclopropylamino}-propionitrile; 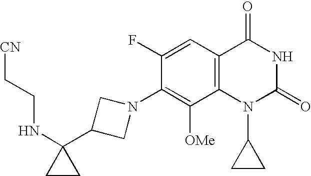
3-{1-[1-(1-Cyclopropyl-6-fluoro-8-methoxy-2,4-dioxo-1,2,3,4-tetrahydro-quinazolin-7-yl)-azetidin-3-yl]-cyclopropylamino}-propionitrile; 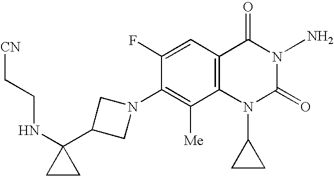
3-{1-[1-(3-Amino-1-cyclopropyl-6-fluoro-8-methyl-2,4-dioxo-1,2,3,4-tetrahydro-quinazolin-7-yl)-azetidin-3-yl]-cyclopropylamino}-propionitrile; 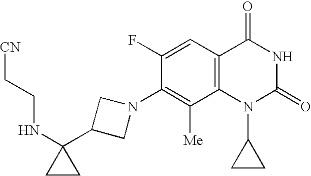
3-{1-[1-(1-Cyclopropyl-6-fluoro-8-methyl-2,4-dioxo-1,2,3,4-tetrahydro-quinazolin-7-yl)-azetidin-3-yl]-cyclopropylamino}-propionitrile; 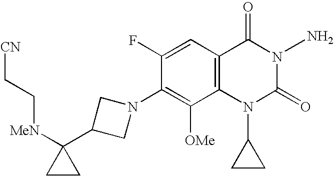
3-({1-[1-(3-Amino-1-cyclopropyl-6-fluoro-8-methoxy-2,4-dioxo-1,2,3,4-tetrahydro-quinazolin-7-yl)-azetidin-3-yl]-cyclopropyl}-methyl-amino)-propionitrile; 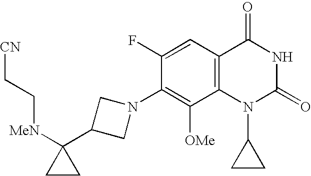
3-({1-[1-(1-Cyclopropyl-6-fluoro-8-methoxy-2,4-dioxo-1,2,3,4-tetrahydro-quinazolin-7-yl)-azetidin-3-yl]-cyclopropyl}-methyl-amino)-propionitrile; 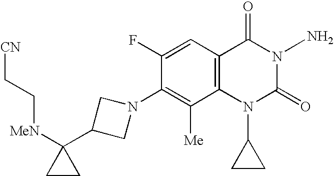
3-({1-[1-(3-Amino-1-cyclopropyl-6-fluoro-8-methyl-2,4-dioxo-1,2,3,4-tetrahydro-quinazolin-7-yl)-azetidin-3-yl]-cyclopropyl}-methyl-amino)-propionitrile; 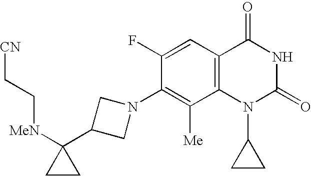
3-({1-[1-(1-Cyclopropyl-6-fluoro-8-methyl-2,4-dioxo-1,2,3,4-tetrahydro-quinazolin-7-yl)-azetidin-3-yl]-cyclopropyl)-methyl-amino)-propionitrile; 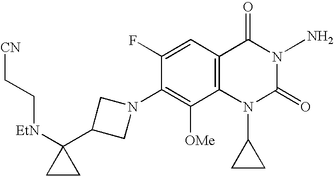
3-({1-[1-(3-Amino-1-cyclopropyl-6-fluoro-8-methoxy-2,4-dioxo-1,2,3,4-tetrahydro-quinazolin-7-yl)-azetidin-3-yl]-cyclopropyl}-ethyl-amino)-propionitrile; 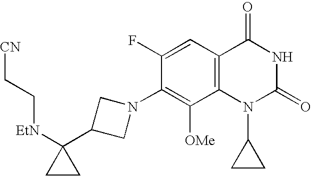
3-({1-[1-(1-Cyclopropyl-6-fluoro-8-methoxy-2,4-dioxo-1,2,3,4-tetrahydro-quinazolin-7-yl)-azetidin-3-yl]-cyclopropyl}-ethyl-amino)-propionitrile; 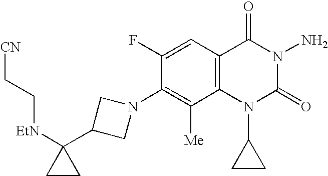
3-({1-[1-(3-Amino-1-cyclopropyl-6-fluoro-8-methyl-2,4-dioxo-1,2,3,4-tetrahydro-quinazolin-7-yl)-azetidin-3-yl]-cyclopropyl}-ethyl-amino)-propionitrile; 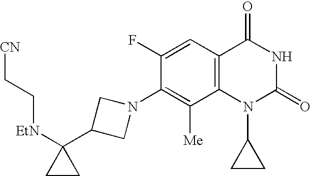
3-({1-[1-(1-Cyclopropyl-6-fluoro-8-methyl-2,4-dioxo-1,2,3,4-tetrahydro-quinazolin-7-yl)-azetidin-3-yl]-cyclopropyl}-ethyl-amino)-propionitrile; 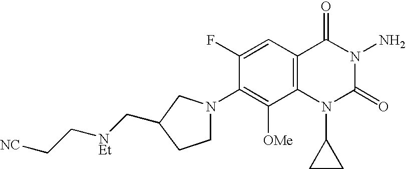
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-ethyl-amino}-propionitrile; 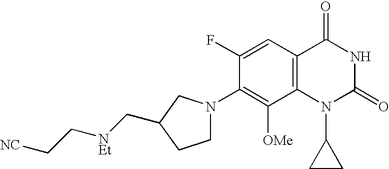
3-{[1-(1-Cyclopropyl-6-fluoro-8-methoxy-2,4-dioxo-1,2,3,4-tetrahydro-quinazolin-7-yl)-pyrrolidin-3-ylmethyl]-ethyl-amino}-propionitrile; 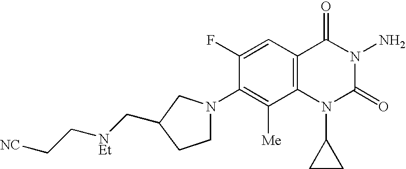
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-ethyl-amino}-propionitrile; 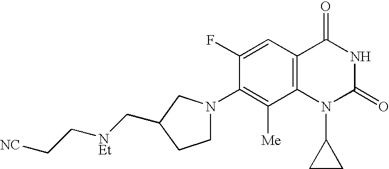
3-{[1-(1-Cyclopropyl-6-fluoro-8-methyl-2,4-dioxo-1,2,3,4-tetrahydro-quinazolin-7-yl)-pyrrolidin-3-ylmethyl]-ethyl-amino}-propionitrile; 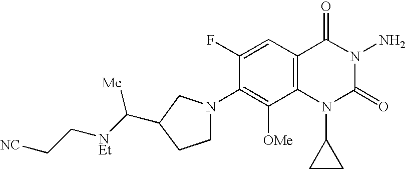
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-ethyl}-ethyl-amino)-propionitrile; 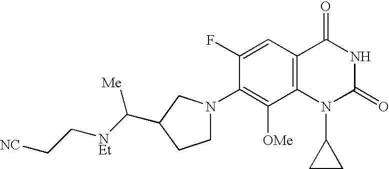
3-({1-[1-(1-Cyclopropyl-6-fluoro-8-methoxy-2,4-dioxo-1,2,3,4-tetrahydro-quinazolin-7-yl)-pyrrolidin-3-yl]-ethyl}-ethyl-amino)-propionitrile; 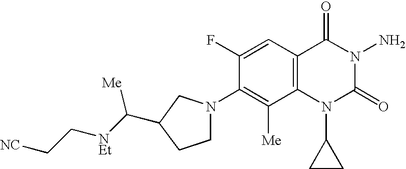
3-({1-[1-(3-Amino-1-cyclopropyl-6-fluoro-8-methyl-2,4-dioxo-1,2,3,4-tetrahydro-quinazolin-7-yl)-pyrrolidin-3-yl]-ethyl}-ethyl-amino)-propionitrile; 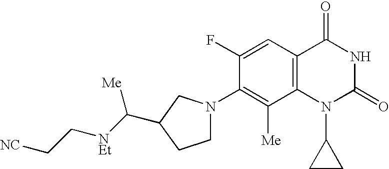
3-({1-[1-(1-Cyclopropyl-6-fluoro-8-methyl-2,4-dioxo-1,2,3,4-tetrahydro-quinazolin-7-yl)-pyrrolidin-3-yl]-ethyl}-ethyl-amino)-propionitrile; 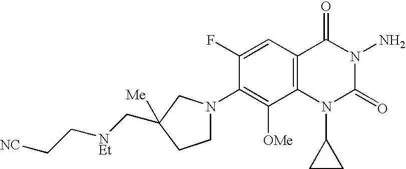
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-ethyl-amino}-propionitrile; 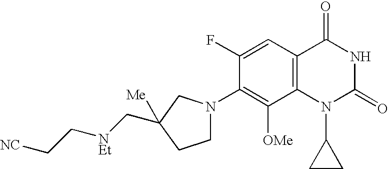
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-ethyl-amino}-propionitrile; 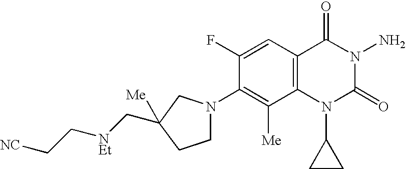
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-ethyl-amino}-propionitrile; 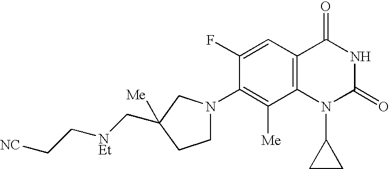
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-ethyl-amino}-propionitrile; 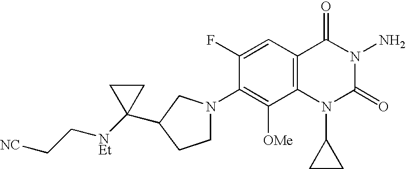
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropyl}-ethyl-amino)-propionitrile; 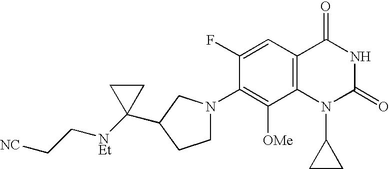
3-({1-[1-(1-Cyclopropyl-6-fluoro-8-methoxy-2,4-dioxo-1,2,3,4-tetrahydro-quinazolin-7-yl)-pyrrolidin-3-yl]-cyclopropyl}-ethyl-amino)-propionitrile; 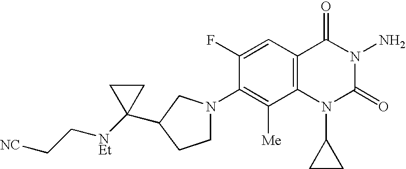
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile; 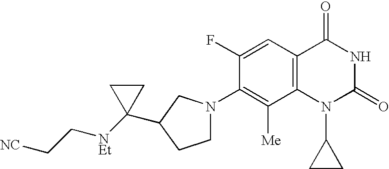
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile; 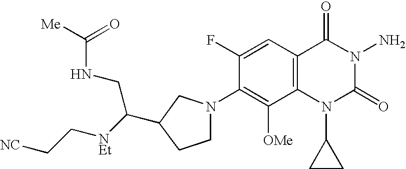
N-{2-[1-(3-Amino-1-cyclopropyl-6-fluoro-8-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide; 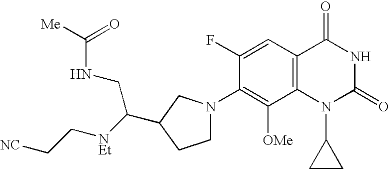
N-{2-[1-(1-Cyclopropyl-6-fluoro-8-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide; 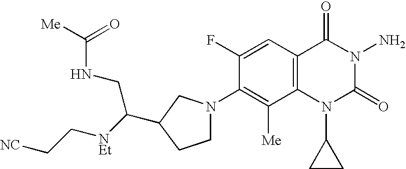
N-{2-[1-(3-Amino-1-cyclopropyl-6-fluoro-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide; 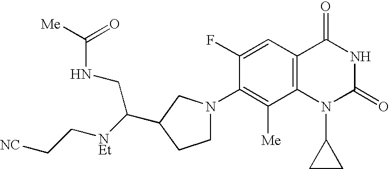
N-{2-[1-(1-Cyclopropyl-6-fluoro-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide; 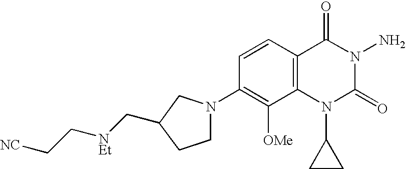
3-{[1-(3-Amino-1-cyclopropyl-8-methoxy-2,4-dioxo-1,2,3,4-tetrahydro-quinazolin-7-yl)-pyrrolidin-3-ylmethyl]-ethyl-amino}-propionitrile; 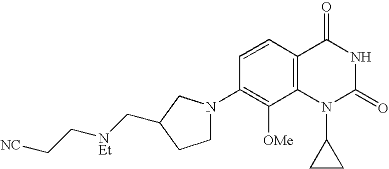
3-{[1-(1-Cyclopropyl-8-methoxy-2,4-dioxo-1,2,3,4-tetrahydro-quinazolin-7-yl)-pyrrolidin-3-ylmethyl]-ethyl-amino}-propionitrile; 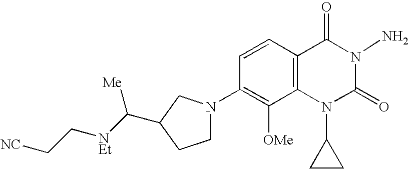
3-({1-[1-(3-Amino-1-cyclopropyl-8-methoxy-2,4-dioxo-1,2,3,4-tetrahydro-quinazolin-7-yl)-pyrrolidin-3-yl]-ethyl}-ethyl-amino)-propionitrile; 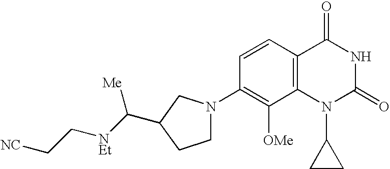
3-({1-[1-(1-Cyclopropyl-8-methoxy-2,4-dioxo-1,2,3,4-tetrahydro-quinazolin-7-yl)-pyrrolidin-3-yl]-ethyl}-ethyl-amino)-propionitrile; 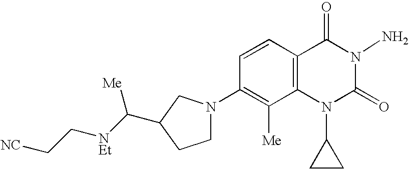
3-({1-[1-(3-Amino-1-cyclopropyl-8-methyl-2,4-dioxo-1,2,3,4-tetrahydro-quinazolin-7-yl)-pyrrolidin-3-yl]-ethyl}-ethyl-amino)-propionitrile; 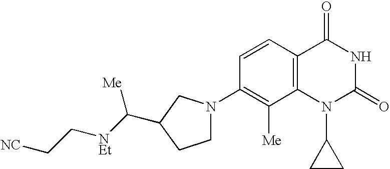
3-({1-[1-(1-Cyclopropyl-8-methyl-2,4-dioxo-1,2,3,4-tetrahydro-quinazolin-7-yl)-pyrrolidin-3-yl]-ethyl}-ethyl-amino)-propionitrile; 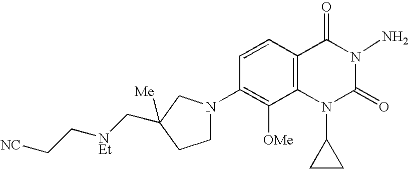
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-ethyl-amino}-propionitrile; 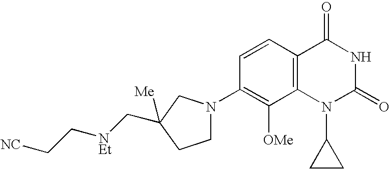
3-{[1-(1-Cyclopropyl-8-methoxy-2,4-dioxo-1,2,3,4-tetrahydro-quinazolin-7-yl)-3-methyl-pyrrolidin-3-ylmethyl]-ethyl-amino}-propionitrile; 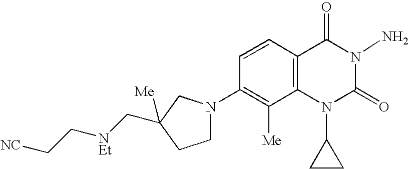
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-ethyl-amino}-propionitrile; 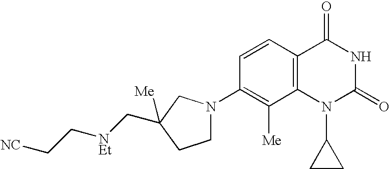
3-{[1-(1-Cyclopropyl-8-methyl-2,4-dioxo-1,2,3,4-tetrahydro-quinazolin-7-yl)-3-methyl-pyrrolidin-3-ylmethyl]-ethyl-amino}-propionitrile; 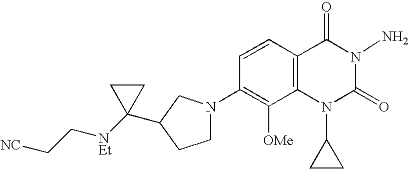
3-({1-[1-(3-Amino-1-cyclopropyl-8-methoxy-2,4-dioxo-1,2,3,4-tetrahydro-quinazolin-7-yl)-pyrrolidin-3-yl]-cyclopropyl}-ethyl-amino)-propionitrile; 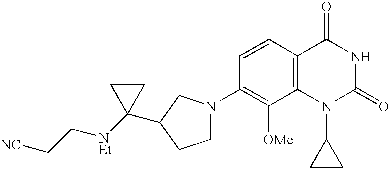
3-({1-[1-(1-Cyclopropyl-8-methoxy-2,4-dioxo-1,2,3,4-tetrahydro-quinazolin-7-yl)-pyrrolidin-3-yl]-cyclopropyl}-ethyl-amino)-propionitrile; 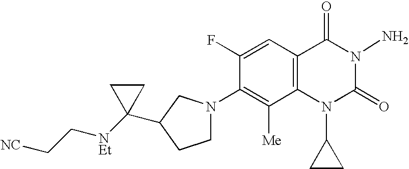
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile; 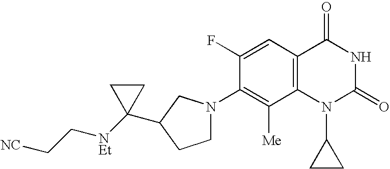
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile; 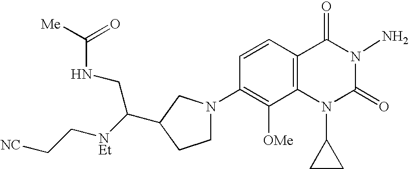
N-{2-[1-(3-Amino-1-cyclopropyl-8-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide; 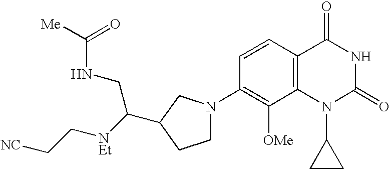
N-{2-[(2-Cyano-ethyl)-ethyl-amino]-2-[1-(1-cyclopropyl-8-methoxy-2,4-dioxo-1,2,3,4-tetrahydro-quinazolin-7-yl)-pyrrolidin-3-yl]-ethyl}-acetamide; 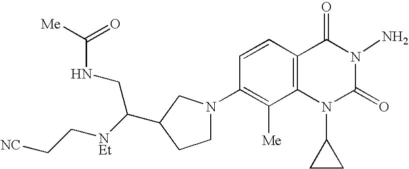
N-{2-[1-(3-Amino-1-cyclopropyl-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide; 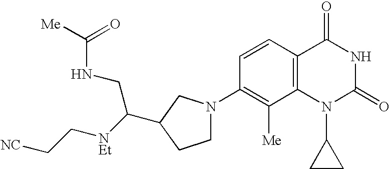
N-{2-[(2-Cyano-ethyl)-ethyl-amino]-2-[1-(1-cyclopropyl-8-methyl-2,4-dioxo-1,2,3,4-tetrahydro-quinazolin-7-yl)-pyrrolidin-3-yl]-ethyl}-acetamide; 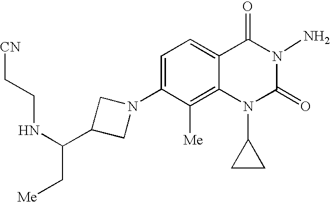
3-{1-[1-(3-Amino-1-cyclopropyl-8-methyl-2,4-dioxo-1,2,3,4-tetrahydro-quinazolin-7-yl)-azetidin-3-yl]-propylamino}-propionitrile; 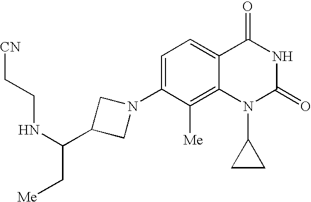
3-{1-[1-(1-Cyclopropyl-8-methyl-2,4-dioxo-1,2,3,4-tetrahydro-quinazolin-7-yl)-azetidin-3-yl]-propylamino}-propionitrile; 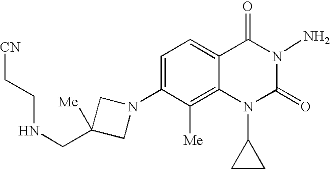
3-{[1-(3-Amino-1-cyclopropyl-8-methyl-2,4-dioxo-1,2,3,4-tetrahydro-quinazolin-7-yl)-3-methyl-azetidin-3-ylmethyl]-amino}-propionitrile; 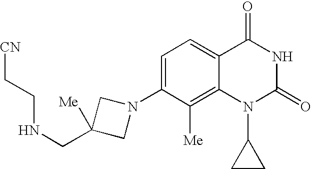
3-{[1-(1-Cyclopropyl-8-methyl-2,4-dioxo-1,2,3,4-tetrahydro-quinazolin-7-yl)-3-methyl-azetidin-3-ylmethyl]-amino}-propionitrile; 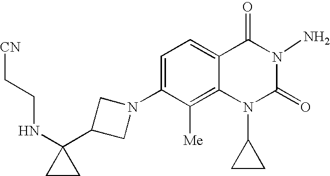
3-{1-[1-(3-Amino-1-cyclopropyl-8-methyl-2,4-dioxo-1,2,3,4-tetrahydro-quinazolin-7-yl)-azetidin-3-yl]-cyclopropylamino}-propionitrile; 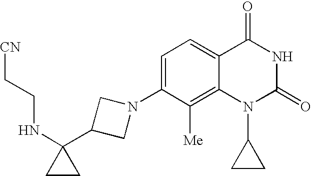
3-{1-[1-(1-Cyclopropyl-8-methyl-2,4-dioxo-1,2,3,4-tetrahydro-quinazolin-7-yl)-azetidin-3-yl]-cyclopropylamino}-propionitrile; 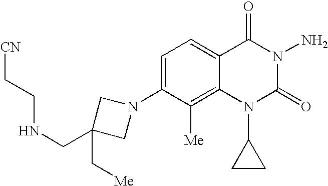
3-{[1-(3-Amino-1-cyclopropyl-8-methyl-2,4-dioxo-1,2,3,4-tetrahydro-quinazolin-7-yl)-3-ethyl-azetidin-3-ylmethyl]-amino}-propionitrile; or 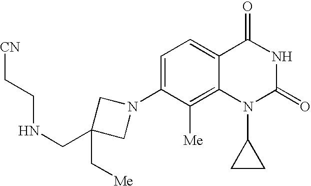
3-{[1-(1-Cyclopropyl-8-methyl-2,4-dioxo-1,2,3,4-tetrahydro-quinazolin-7-yl)-3-ethyl-azetidin-3-ylmethyl]-amino}-propionitrile.

A compound of formula IV: 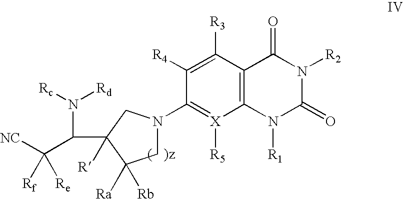
or a pharmaceutically acceptable salt thereof, wherein:
X is N or C, provided that when X is N, R₅ is absent at that position;
n is 0, 1, or 2;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
   CH₂(C₃-C₆)cycloalkyl;
R₂ is H,
   NH₂, 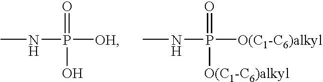
   NH(C₁-C₆)alkyl,
   NH(C₃-C₆)cycloalkyl,
   NH-aryl,
   NH-heteroaryl,
   NHSO₂—(C₁-C₆)alkyl,
   NHSO₂-aryl,
   NHSO₂-heteroaryl, 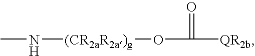
   wherein g is an integer from 1 to 10, Q is O, NH, or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 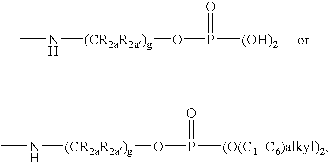
   wherein R₂ₐ and R_{2a′} are as defined above, 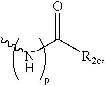
   wherein [symbol] indicates the point of attachment, p is 0 or 1, and
      R_{2C} is H,
         (C₁-C₆)alkyl,
         O(C₁-C₆)alkyl,
         (C₃-C₇)cycloalkyl,
         aryl,
         heterocyclo,
         heteroaryl, or 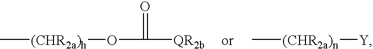
         wherein R₂ₐ, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 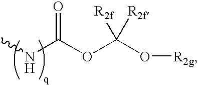
         wherein q is 0 or 1, R_{2f} and R_{2f ′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy
   nitrile; or
R₁ , and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
z is 0, 1, or 2;
Rₐ and R_{b} are each independently H, (C₁-C₆)alkyl, (C₁-C₆)alkoxy, halo(C₁-C₆)alkyl, halo, or Rₐ and R_{b} taken together with the carbon to which they are attached form a 3, 4, 5 or 6-membered substituted or unsubstituted ring;
R′ is H,
   halo,
   (C₁-C₆)alkyl,
   O(C₁-C₆)alkyl
   halo(C₁-C₆)alkyl,
   aryl, or
   heteroaryl;
R_{c} and R_{d} are each independently H,
   (C₁-C₆)alkylnitrile; 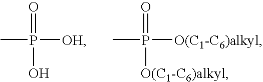
   (C₁-C₆)alkyl,
   (C₃-C₆)cycloalkyl,
   heteroaryl,
   SO₂—(C₁-C₆)alkyl,
   SO₂-aryl,
   SO₂-heteroaryl, 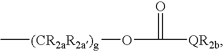
   wherein g is an integer from 1 to 10, Q is as defined above, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 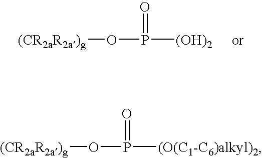
   wherein R₂ₐ and R_{2a′} are as defined above, 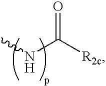
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 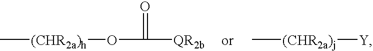
         wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 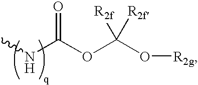
         wherein q is 0 or 1, R_{2f} and R_{2f ′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
         heteroaryl; and
Rₑ and R_{f} are each independently H, C₁-C₆ alkyl, haloalkyl, halo, or Rₑ and R_{f} taken together with the carbon to which they are attached form a 3, 4, 5 or 6-membered substituted or unsubstituted ring.

What is also provided is a compound which is: 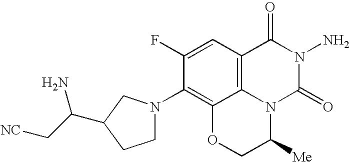
3-Amino-3-[1-(5-amino-8-fluoro-3-methyl-4,6-dioxo-2,3,5,6-tetrahydro-4H-1-oxa-3a,5-diaza-phenalen-9-yl)-pyrrolidin-3-yl]-propionitrile; 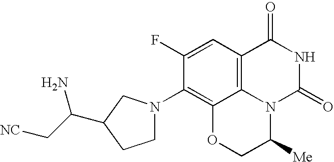
3-Amino-3-[1-(8-fluoro-3-methyl-4,6-dioxo-2,3,5,6-tetrahydro-4H-1-oxa-3a,5-diaza-phenalen-9-yl)-pyrrolidin-3-yl]-propionitrile; 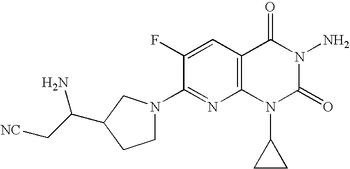
3-Amino-3-[1-(3-amino-1-cyclopropyl-6-fluoro-2,4-dioxo-1,2,3,4-tetrahydro-pyrido[2,3-d]pyrimidin-7-yl)-pyrrolidin-3-yl]-propionitrile; 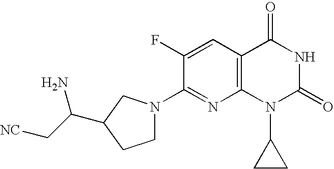
3-Amino-3-[1-(1-cyclopropyl-6-fluoro-2,4-dioxo-1,2,3,4-tetrahydro-pyrido[2,3-d]pyrimidin-7-yl)-pyrrolidin-3-yl]-propionitrile; 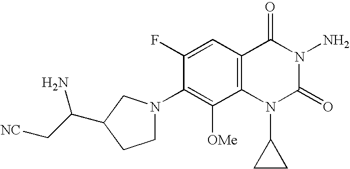
3-Amino-3-[1-(3-amino-1-cyclopropyl-6-fluoro-8-methoxy-2,4-dioxo-1,2,3,4-tetrahydro-quinazolin-7-yl)-pyrrolidin-3-yl]-propionitrile; 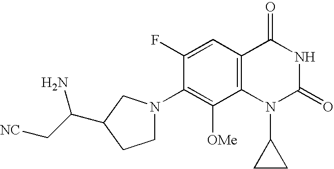
3-Amino-3-[1-(1-cyclopropyl-6-fluoro-8-methoxy-2,4-dioxo-1,2,3,4-tetrahydro-quinazolin-7-yl)-pyrrolidin-3-yl]-propionitrile; 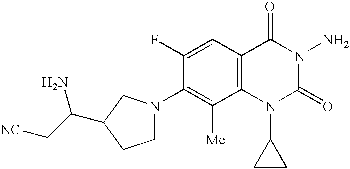
3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-propionitrile; 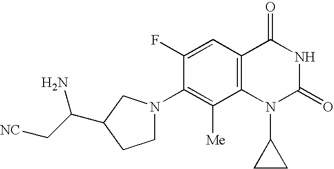
3-Amino-3-[1-(1-cyclopropyl-6-fluoro-8-methyl-2,4-dioxo-1,2,3,4-tetrahydro-quinazolin-7-yl)-pyrrolidin-3-yl]-propionitrile; 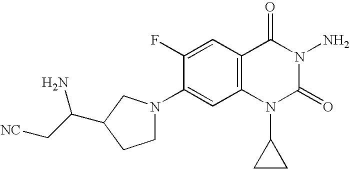
3-Amino-3-[1-(3-amino-1-cyclopropyl-6-fluoro-2,4-dioxo-1,2,3,4-tetrahydro-quinazolin-7-yl)-pyrrolidin-3-yl]-propionitrile; 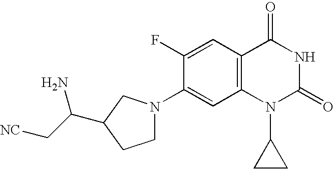
b 3-Amino-3-[1-(1-cyclopropyl-6-fluoro-2,4-dioxo-1,2,3,4-tetrahydro-quinazolin-7-yl)-pyrrolidin-3-yl]-propionitrile; 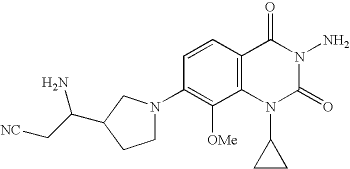
3-Amino-3-[1-(3-amino-1-cyclopropyl-8-methoxy-2,4-dioxo-1,2,3,4-tetrahydro-quinazolin-7-yl)-pyrrolidin-3-yl]-propionitrile; 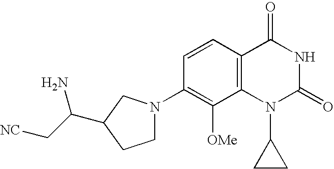
3-Amino-3-[1-(1-cyclopropyl-8-methoxy-2,4-dioxo-1,2,3,4-tetrahydro-quinazolin-7-yl)-pyrrolidin-3-yl]-propionitrile; 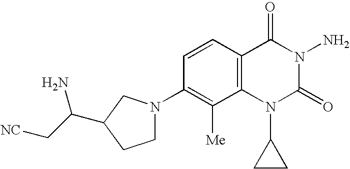
3-Amino-3-[1-(3-amino-1-cyclopropyl-g-methyl-2,4-oxo-1,2,3,4-tetrahydro-quinazolin-7-yl)-pyrrolidin-3-yl]-propionitrile; 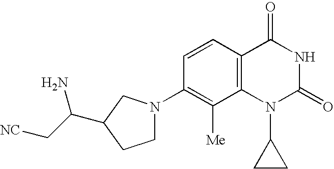
3-Amino-3-[1-(1-cyclopropyl-8-methyl-2,4-dioxo-1,2,3,4-tetrahydro-quinazolin-7-yl)-pyrrolidin-3-yl]-propionitrile; 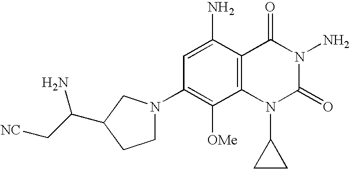
3-Amino-3-[1-(3,5-diamino-1-cyclopropyl-8-methoxy-2,4-dioxo-1,2,3,4-tetrahydro-quinazolin-7-yl)-pyrrolidin-3-yl]-propionitrile; 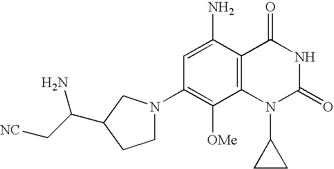
3-Amino-3-[1-(5-amino-1-cyclopropyl-8-methoxy-2,4-dioxo-1,2,3,4-tetrahydro-quinazolin-7-yl)-pyrrolidin-3-yl]-propionitrile; 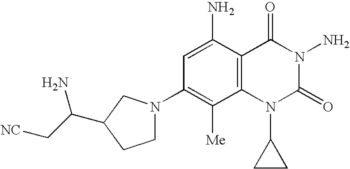
3-Amino-3-[1-(3,5-diamino-1-cyclopropyl-8-methyl-2,4-dioxo-1,2,3,4-tetrahydro-quinazolin-7-yl)-pyrrolidin-3-yl]-propionitrile; 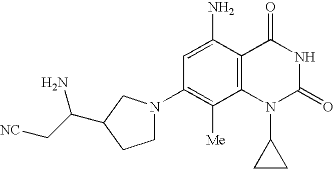
3-Amino-3-[1-(5-amino-1-cyclopropyl-8-methyl-2,4-dioxo-1,2,3,4-tetrahydro-quinazolin-7-yl)-pyrrolidin-3-yl]-propionitrile; 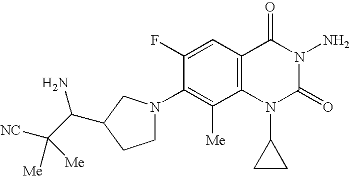
3-Amino-3-[1-(3-amino-1-cyclopropyl-6-fluoro-8-methoxy-2,4-dioxo-1,2,3,4-tetrahydro-quinazolin-7-yl)-pyrrolidin-3-yl]-2,2-dimethyl-propionitrile; 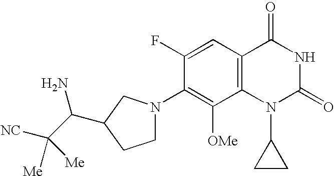
3-Amino-3-[1-(1-Cyclopropyl-6-fluoro-8-methoxy-2,4-dioxo-1,2,3,4-tetrahydro-quinazolin-7-yl)-pyrrolidin-3-yl]-2,2-dimethyl-propionitrile; 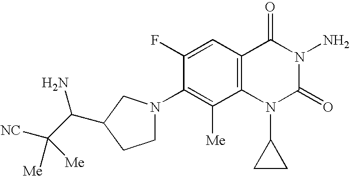
3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-2,2-dimethyl-propionitrile; 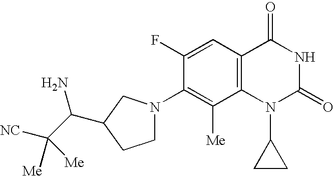
3-Amino-3-[1-(1-cyclopropyl-6-fluoro-8-methyl-2,4-dioxo-1,2,3,4-tetrahydro-quinazolin-7-yl)-pyrrolidin-3-yl]-2,2-dimethyl-propionitrile; 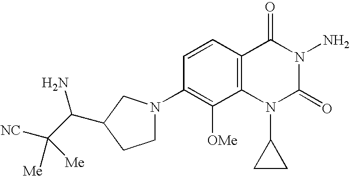
3-Amino-3-[1-(3-amino-1-cyclopropyl-8-methoxy-2,4-dioxo-1,2,3,4-tetrahydro-quinazolin-7-yl)-pyrrolidin-3-yl]-2,2-dimethyl-propionitrile; 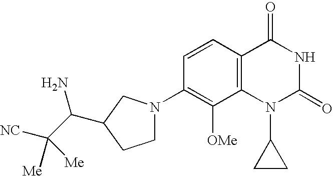
3-Amino-3-[1-(1-cyclopropyl-8-methoxy-2,4-dioxo-1,2,3,4-tetrahydro-quinazolin-7-yl)-pyrrolidin-3-yl]-2,2-dimethyl-propionitrile; 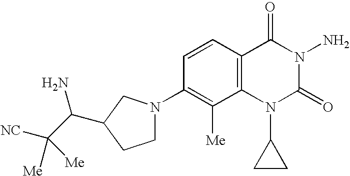
3-Amino-3-[1-(3-amino-1-cyclopropyl-8-methyl-2,4-dioxo-1,2,3,4-tetrahydro-quinazolin-7-yl)-pyrrolidin-3-yl]-2,2-dimethyl-propionitrile; 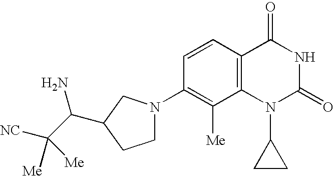
3-Amino-3-[1-(1-cyclopropyl-8-methyl-2,4-dioxo-1,2,3,4-tetrahydro-quinazolin-7-yl)-pyrrolidin-3-yl]-2,2-dimethyl-propionitrile; 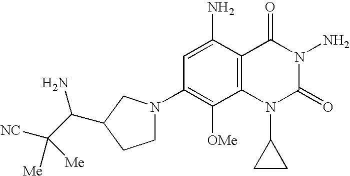
3-Amino-3-[1-(3,5-diamino-1-cyclopropyl-8-methoxy-2,4-dioxo-1,2,3,4-tetrahydro-quinazolin-7-yl)-pyrrolidin-3-yl]-2,2-dimethyl-propionitrile; 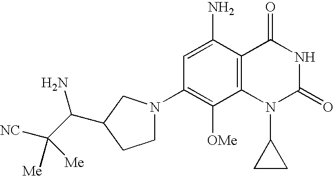
3-Amino-3-[1-(5-amino-1-cyclopropyl-8-methoxy-2,4-dioxo-1,2,3,4-tetrahydro-quinazolin-7-yl)-pyrrolidin-3-yl]-2,2-dimethyl-propionitrile; 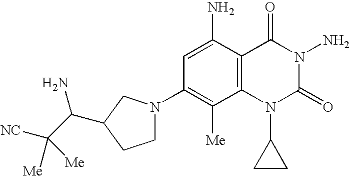
3-Amino-3-[1-(3,5-diamino-1-cyclopropyl-8-methyl-2,4-dioxo-1,2,3,4-tetrahydro-quinazolin-7-yl)-pyrrolidin-3-yl]-2,2-dimethyl-propionitrile; 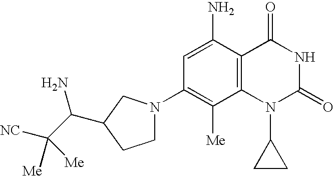
3-Amino-3-[1-(5-amino-1-cyclopropyl-8-methyl-2,4-dioxo-1,2,3,4-tetrahydro-quinazolin-7-yl)-pyrrolidin-3-yl]-2,2-dimethyl-propionitrile; 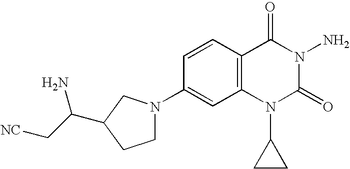
3-Amino-3-[1-(3-amino-1-cyclopropyl-2,4-dioxo-1,2,3,4-tetrahydro-quinazolin-7-yl)-pyrrolidin-3-yl]-propionitrile; 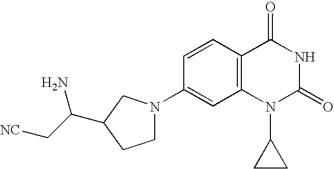
3-Amino-3-[1-(1-cyclopropyl-2,4-dioxo-1,2,3,4-tetrahydro-quinazolin-7-yl)-pyrrolidin-3-yl]-propionitrile; 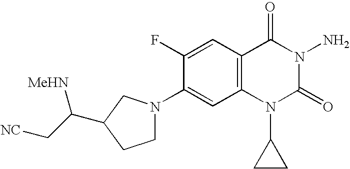
3-[1-(3-Amino-1-cyclopropyl-6-fluoro-2,4-dioxo-1,2,3,4-tetrahydro-quinazolin-7-yl)-pyrrolidin-3-yl]-3-methylamino-propionitrile; 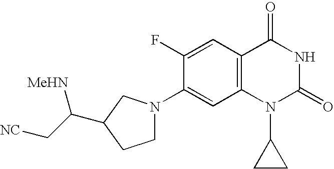
3-[1-(1-cyclopropyl-6-fluoro-2,4-dioxo-1,2,3,4-tetrahydro-quinazolin-7-yl)-pyrrolidin-3-yl]-3-methylamino-propionitrile; 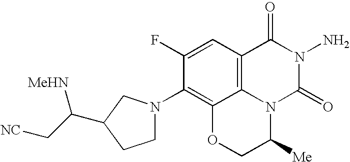
3-[1-(5-Amino-8-fluoro-3-methyl-4,6-dioxo-2,3,5,6-tetrahydro-4H-1-oxa-3a,5-diaza-phenalen-9-yl)-pyrrolidin-3-yl]-3-methylamino-propionitrile; 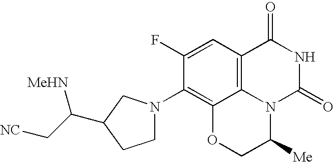
3-[1-(8-Fluoro-3-methyl-4,6-dioxo-2,3,5,6-tetrahydro-4H-1-oxa-3a,5-diaza-phenalen-9-yl)-pyrrolidin-3-yl]-3-methylamino-propionitrile; 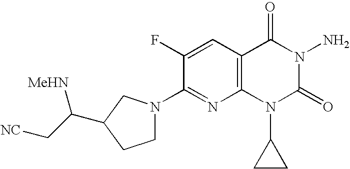
3-[1-(3-Amino-1-cyclopropyl-6-fluoro-2,4-dioxo-1,2,3,4-tetrahydro-pyrido[2,3-d]pyrimidin-7-yl)-pyrrolidin-3-yl]-3-methylamino-propionitrile; 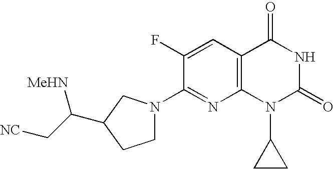
3-[1-(1-cyclopropyl-6-fluoro-2,4-dioxo-1,2,3,4-tetrahydro-pyrido[2,3-d]pyrimidin-7-yl)-pyrrolidin-3-yl]-3-methylamino-propionitrile; 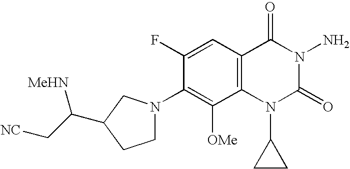
3-[1-(3-Amino-1-cyclopropyl-6-fluoro-8-methoxy-2,4-dioxo-1,2,3,4-tetrahydro-quinazolin-7-yl)-pyrrolidin-3-yl]-3-methylamino-propionitrile; 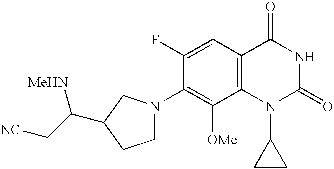
3-[1-(1-cyclopropyl-6-fluoro-8-methoxy-2,4-dioxo-1,2,3,4-tetrahydro-quinazolin-7-yl)-pyrrolidin-3-yl]-3-methylamino-propionitrile; 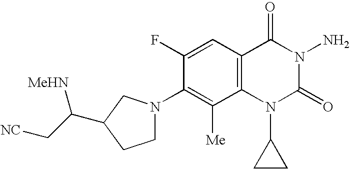
3-[1-(3-Amino-1-cyclopropyl-6-fluoro-8-methyl-2,4-dioxo-1,2,3,4-tetrahydro-quinazolin-7-yl)-pyrrolidin-3-yl]-3-methylamino-propionitrile; 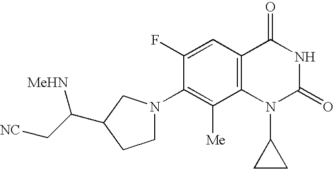
3-[1-(1-cyclopropyl-6-fluoro-8-methyl-2,4-dioxo-1,2,3,4-tetrahydro-quinazolin-7-yl)-pyrrolidin-3-yl]-3-methylamino-propionitrile; 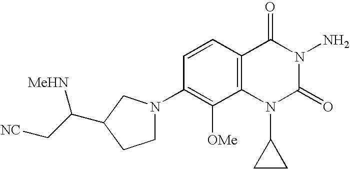
3-[1-(3-Amino-1-cyclopropyl-8-methoxy-2,4-dioxo-1,2,3,4-tetrahydro-quinazolin-7-yl)-pyrrolidin-3-yl]-3-methylamino-propionitrile; 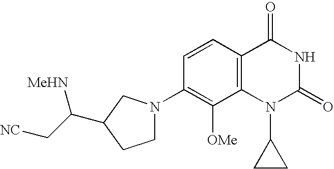
3-[1-(1-cyclopropyl-8-methoxy-2,4-dioxo-1,2,3,4-tetrahydro-quinazolin-7-yl)-pyrrolidin-3-yl]-3-methylamino-propionitrile; 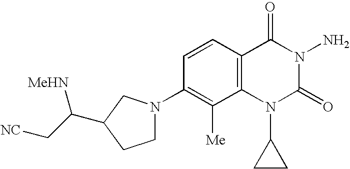
3-[1-(3-Amino-1-cyclopropyl-8-methyl-2,4-dioxo-1,2,3,4-tetrahydro-quinazolin-7-yl)-pyrrolidin-3-yl]-3-methylamino-propionitrile; 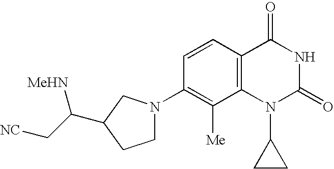
3-[1-(1-cyclopropyl-8-methyl-2,4-dioxo-1,2,3,4-tetrahydro-quinazolin-7-yl)-pyrrolidin-3-yl]-3-methylamino-propionitrile; 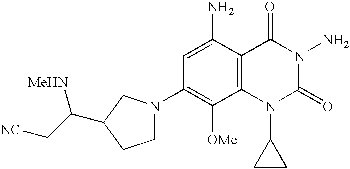
3-[1-(3,5-Diamino-1-cyclopropyl-8-methoxy-2,4-dioxo-1,2,3,4-tetrahydro-quinazolin-7-yl)-pyrrolidin-3-yl]-3-methylamino-propionitrile; 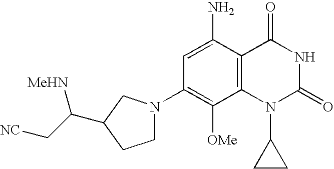
3-[1-(5-amino-1-cyclopropyl-8-methoxy-2,4-dioxo-1,2,3,4-tetrahydro-quinazolin-7-yl)-pyrrolidin-3-yl]-3-methylamino-propionitrile; 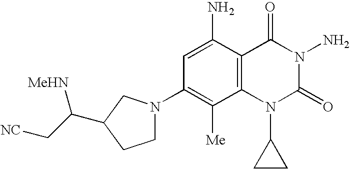
3-[1-(3,5-Diamino-1-cyclopropyl-8-methyl-2,4-dioxo-1,2,3,4-tetrahydro-quinazolin-7-yl)-pyrrolidin-3-yl]-3-methylamino-propionitrile; 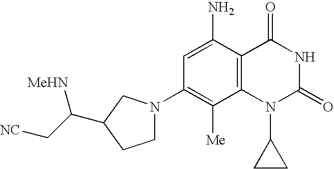
3-[1-(5-amino-1-cyclopropyl-8-methyl-2,4-dioxo-1,2,3,4-tetrahydro-quinazolin-7-yl)-pyrrolidin-3-yl]-3-methylamino-propionitrile; 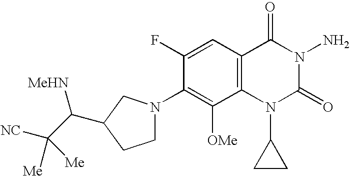
3-[1-(3-Amino-1-cyclopropyl-6-fluoro-8-methoxy-2,4-dioxo-1,2,3,4-tetrahydro-quinazolin-7-yl)-pyrrolidin-3-yl]-2,2-dimethyl-3-methylamino-propionitrile; 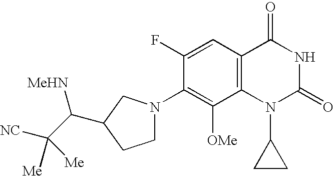
3-[1-(1-Cyclopropyl-6-fluoro-8-methoxy-2,4-dioxo-1,2,3,4-tetrahydro-quinazolin-7-yl)-pyrrolidin-3-yl]-2,2-dimethyl-3-methylamino-propionitrile; 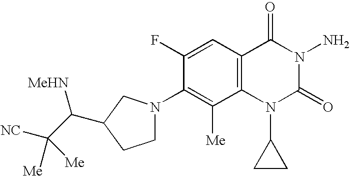
3-[1-(3-Amino-1-cyclopropyl-6-fluoro-8-methyl-2,4-dioxo-1,2,3,4-tetrahydro-quinazolin-7-yl)-pyrrolidin-3-yl]-2,2-dimethyl-3-methylamino-propionitrile; 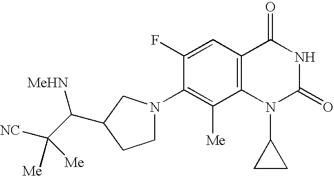
3-[1-(1-Cyclopropyl-6-fluoro-8-methyl-2,4-dioxo-1,2,3,4-tetrahydro-quinazolin-7-yl)-pyrrolidin-3-yl]-2,2-dimethyl-3-methylamino-propionitrile; 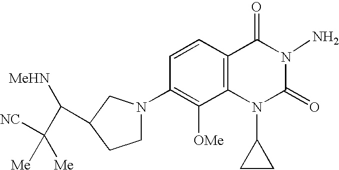
3-[1-(3-Amino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile; 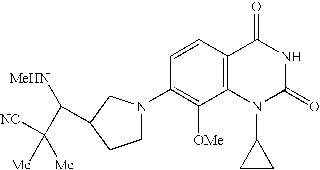
3-[1-(1-Cyclopropyl-8-methoxy-2,4-dioxo-1,2,3,4-tetrahydro-quinazolin-7-yl)-pyrrolidin-3-yl]-2,2-dimethyl-3-methylamino-propionitrile; 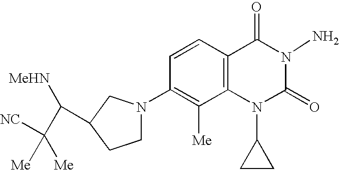
3-[1-(3-Amino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile; 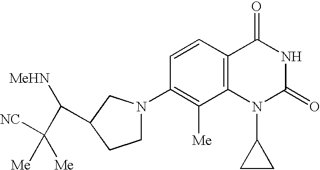
3-[1-(1-Cyclopropyl-8-methyl-2,4-dioxo-1,2,3,4-tetrahydro-quinazolin-7-yl)-pyrrolidin-3-yl]-2,2-dimethyl-3-methylamino-propionitrile; 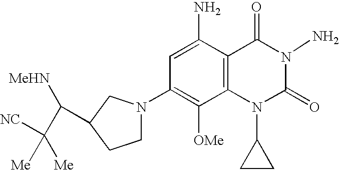
3-[1-(3,5-Diamino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile; 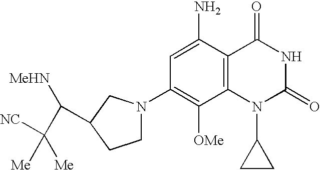
3-[1-(5-amino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile; 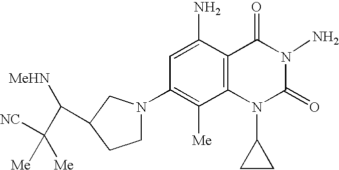
3-[1-(3,5-Diamino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile; or 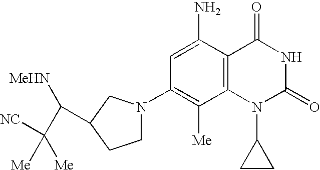
3-[1-(5-amino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile.

What is also provided is a compound of formula V or VI 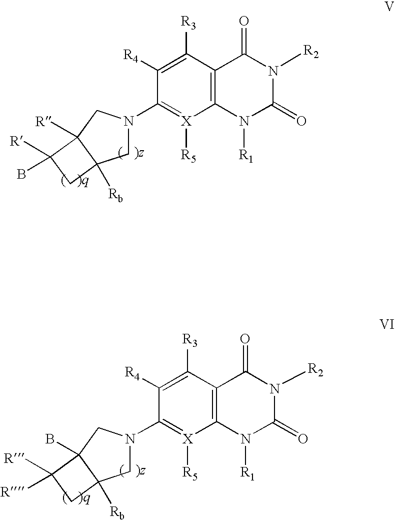
or a pharmaceutically acceptable salt thereof, wherein:
X is N or C, provided that when X is N, R₅ is absent at that position;
n and q are each independently 0, 1, or 2;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
   CH₂(C₃-C₆)cycloalkyl;
R₂ is H,
   NH₂, 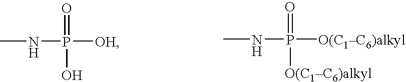
   NH(C₁-C₆)alkyl,
   NH(C₃-C₆)cycloalkyl,
   NH-aryl,
   NH-heteroaryl,
   NHSO₂—(C₁-C₆)alkyl,
   NHSO₂-aryl,
   NHSO₂-heteroaryl, 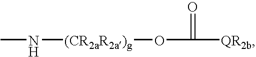
   wherein g is an integer from 1 to 10, Q is O, NH, or is absent, and R_{2a′} and R₂ₐ are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 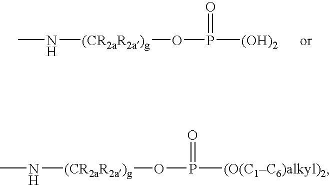
   wherein Rₐ and R_{2a′} are as defined above, 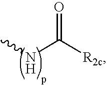
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 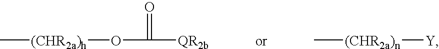
         wherein R₂ₐ, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 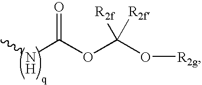
         wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy
   nitrile; or
R₁ and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
q is 0, 1, 2, or 3 and z is 0, 1, or 2;
R_{b} is H, (C₁-C₆)alkyl, halo(C₁-C₆)alkyl, halo, or Rₐ and R_{b} taken together with the carbon to which they are attached form a 3, 4, 5 or 6-membered substituted or unsubstituted ring;
R′, R″, R′″, and R″″ are each independently H,
   (C₁-C₆) alkyl,
   —O(C₁-C₆)alkyl,
   halo(C₁-C₆)alkyl,
   aryl, or
   heteroaryl;
B is 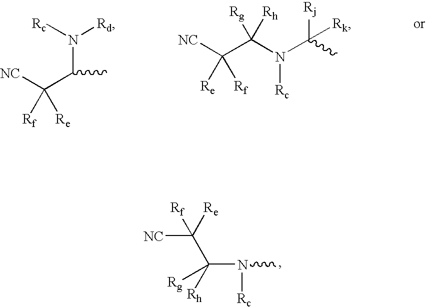
provided that when B is 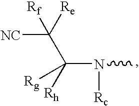
R′ is not O(C₁-C₆)alkyl, and wherein [symbol] indicates the point of attachment, and
R_{c} and R_{d} are each independently H,
   (C₁-C₆)alkylnitrile; 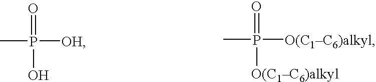
   (C₁-C₆)alkyl,
   (C₃-C₆)cycloalkyl,
   heteroaryl,
   SO₂—(C₁-C₆)alkyl,
   SO₂-aryl,
   SO₂-heteroaryl, 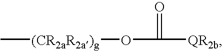
   wherein g is an integer from 1 to 10, Q is as defined above, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 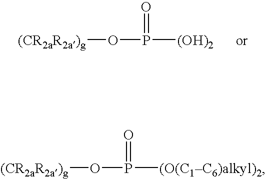
   wherein R₂ₐ and R_{2a′} are as defined above, 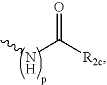
   wherein [symbol] indicates the point of attachment, p is 0 or 1, and
      R_{2c} is H,
         (C₁-C₆)alkyl,
         O(C₁-C₆)alkyl,
         (C₃-C₇)cycloalkyl,
         aryl,
         heterocyclo,
         heteroaryl, or 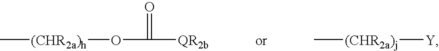
         wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 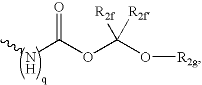
         wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
         (C₁-C₆)alkyl,
         (C₃-C₇)cycloalkyl,
         aryl, or
         heterocyclo, or
         heteroaryl;
Rₑ and R_{f} are each independently H, C₁-C₆ alkyl, haloalkyl, halo, or Rₑ and R_{f} taken together with the carbon to which they are attached form a 3, 4, 5 or 6-membered substituted or unsubstituted ring;
R_{g} and Rₕ are each independently H, C₁-C₆ alkyl, haloalkyl, or taken together with the carbon to which they are attached to form a 3, 4, 5 or 6-membered substituted or unsubstituted ring; and
Rⱼ and Rₖ are each independently H, (C₁-C₆)alkyl, haloalkyl, (C₁-C₆)alkyl-NR_{c}R_{d}, (C₁-C₆)alkyl-OR_{c}, aryl, heteroaryl, heterocycle, 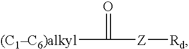
wherein Z is O or NR_{c}, or Rⱼ and Rₖ taken together with the carbon to which they are attached to form a 3, 4, 5 or 6-membered substituted or unsubstituted ring.

What is also provided is a compound which is 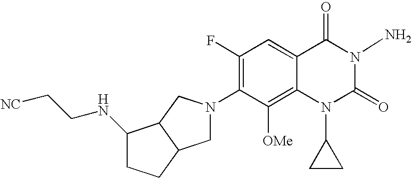
3-[2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile; 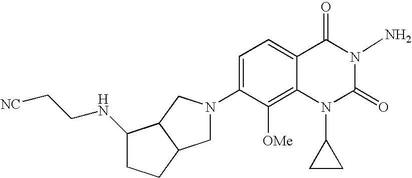
3-[2-(3-Amino-1-cyclopropyl-8-methoxy-2,4-dioxo-1,2,3,4-tetrahydro-quinazolin-7-yl)-octahydro-cyclopenta[c]pyrrol-4-ylamino]-propionitrile; 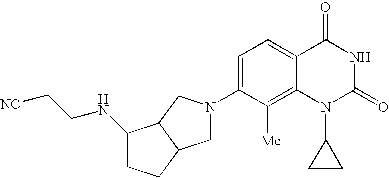
3-[2-(1-Cyclopropyl-6-fluoro-8-methyl-2,4-dioxo-1,2,3,4-tetrahydro-quinazolin-7-yl)-octahydro-cyclopenta[c]pyrrolyl-4-ylamino]-propionitrile; 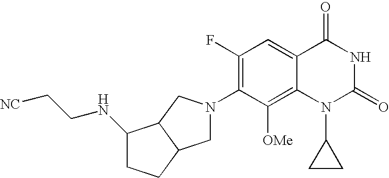
3-[2-(1-Cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile; 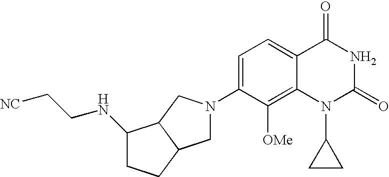
3-[2-(1-Cyclopropyl-8-methoxy-2,4-dioxo-1,2,3,4-tetrahydro-quinazolin-7-yl)-octahydro-cyclopenta[c]pyrrol-4-ylamino]-propionitrile; or 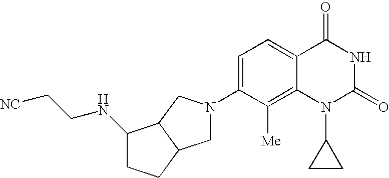
3-[2-(1-Cyclopropyl-8-methyl-2,4-dioxo-1,2,3,4-tetrahydro-quinazolin-7-yl)-octahydro-cyclopenta[c]pyrrol-4-ylamino]-propionitrile.

What is also provided is a pharmaceutical formulation comprising a compound of one of formula I admixed with a pharmaceutically acceptable diluent, carrier, or excipient.

What is also provided is a method of treating a bacterial infection in a mammal, comprising administering to a mammal in need thereof an effective amount of a compound of formula I.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a linear or branched hydrocarbon of from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substitutents selected from lower (C₁-C₆)alkoxy, (C₁-C₆)thioalkoxy, halogen, aryl, heteroaryl, oxo, thio, —OH, —SH, —F, —CF₃, —OCF₃, —NO₂, —CO₂H, —CO₂(C₁-C₆)alkyl, or

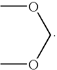

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substitutents selected from alkyl, alkoxy, thioalkoxy, hydroxy, thiol, halogen, formyl, carboxyl, —CO₂(C₁-C₆)alkyl, —CO(C₁-C₆)alkyl, aryl, heteroaryl, wherein alkyl, aryl, and heteroaryl are as defined herein, or as indicated above for alkyl. Examples of substituted cycloalkyl groups include fluorocyclopropyl.

The term “halo” includes chlorine, fluorine, bromine, and iodine.

The term “haloalkyl” means a (C₁-C₆)alkyl group substituted with one or more halo.

The term “aryl” means a cyclic or polycyclic aromatic ring having from 5 to 12 carbon atoms, and being unsubstituted or substituted with one or more of the substitutent groups recited above for alkyl groups including, halogen, nitro, cyano —OH, —SH, —F, —CF₃, —OCF₃ 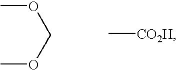
—CO₂C₁-C₆ alkyl, or —SO₂alkyl. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4,5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substitutents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substitutents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophenyl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

In some cases, to prepare the compounds of the invention disclosed herein, protecting groups may have been used to allow synthetic manipulation of one functional group in the presence of other functional groups. The appropriate use and choice of protecting groups is well-known by one skilled in the art. It is also to be understood that such groups not only serve to protect chemically reactive sites, but also to enhance solubility or otherwise change physical properties. A good general reference for protecting group preparation and deprotection is Greene, Theodora, Protective Groups in Organic Synthesis; Wiley: New York, USA, 1991 and later editions. Thus, it is to be further understood that invention compounds characterized by the presence of a protecting group as disclosed and described in Greene are also to be considered invention compounds.

When a bond is represented by a symbol such as [symbol] this is meant to represent that the bond may be absent or present provided that the resultant compound is stable and of satisfactory valency.

When a bond is represented by a line such as [symbol] this is meant to represent that the bond is the point of attachment between two molecular subunits.

The term “patient” means all mammals, including humans. Other examples of patients include cows, dogs, cats, goats, sheep, pigs, and rabbits.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of the invention are also useful as intermediates for preparing other compounds of the invention. Thus, a compound wherein R₂ is BF₂, can be hydrolyzed to form another compound of the invention wherein R₂ is H.

Some of the compounds of Formula I are capable of further forming pharmaceutically acceptable acid-addition and/or base salts. All of these forms are within the scope of the present invention. Thus, pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, S. M. et. al., “Pharmaceutical Salts,” Journal of Pharmaceutical Science, 1977; 66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is an inactive derivative of a drug molecule that requires a chemical or an enzymatic biotransformation in order to release the active parent drug in the body.

Specific and preferred values for the compounds of the present invention are listed below for radicals, substitutents, and ranges are for illustration purposes only, and they do not exclude other defined values or other values within defined ranges for the radicals and substitutents.

Unless otherwise stated or defined, abbreviations used herein conform to the style sheet of American Chemical Society journals.

Thus, we turn now to a compound of formula I, which has the structure:

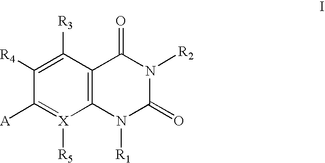

A specific value for X is N or C—OMe or C-Me. A specific value for R₁ is (C₁-C₆)cycloalkyl and halo(C₁-C₆)cycloalkyl, aryl, or heteroaryl. A specific value for R₂ is H, NH₂, 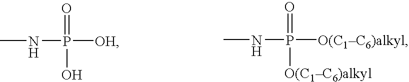
NH(C₁-C₆)alkyl, NHSO₂-(C₁-C₆)alkyl, NHSO₂-aryl, or NHSO₂-heteroaryl. A specific value for R₃ is H, Me, or NH₂. A specific value for R₄ is H or halo. A specific value for R₅ is halo, methyl, trifluoromethyl, methoxy, fluoromethoxy, difluoromethoxy, or trifluoromethoxy.

In another embodiment of a compound of formula I, a specific value for X is C—OMe or C-Me. A specific value for R₁ is (C₁-C₆)cycloalkyl and halo(C₁-C₆)cycloalkyl, aryl, or heteroaryl. A specific value for R₂ is H, NH₂, 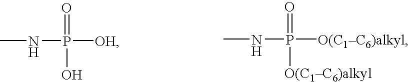
NHSO₂—(C₁-C₆)alkyl, NHSO₂-aryl, or NHSO₂-heteroaryl. A specific value for R₃ is H, Me, or NH₂. A specific value for R₄ is H or F. A specific value for X is C or N. A specific value for R₅ is methyl, methoxy, or chloro.

In another embodiment of a compound of formula I, R₁, R₂, R₃, and R₅ are as provided in the following structures, R₁, R₃, and R₅ are as provided in the following structures, and R₂ is NH₂ or H, R₄ is H or F, and A is

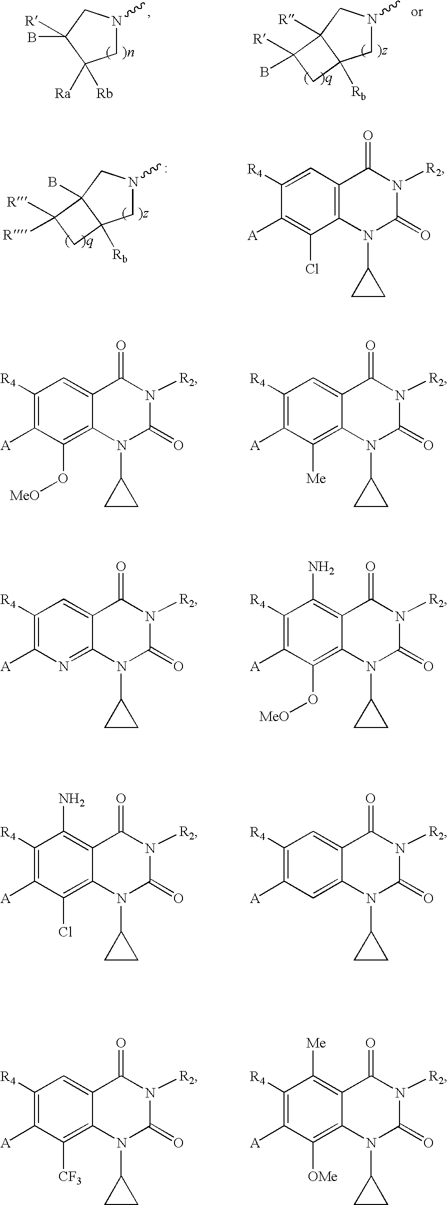

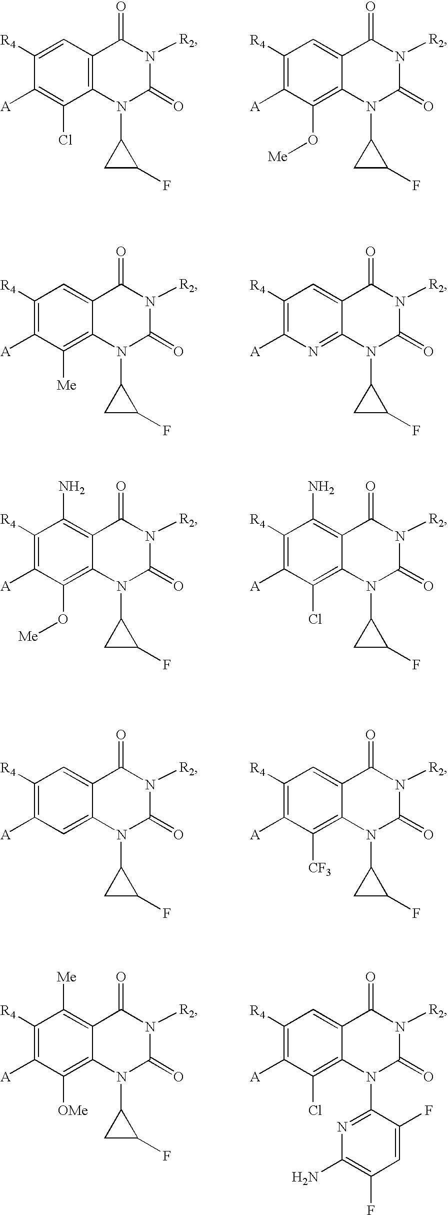

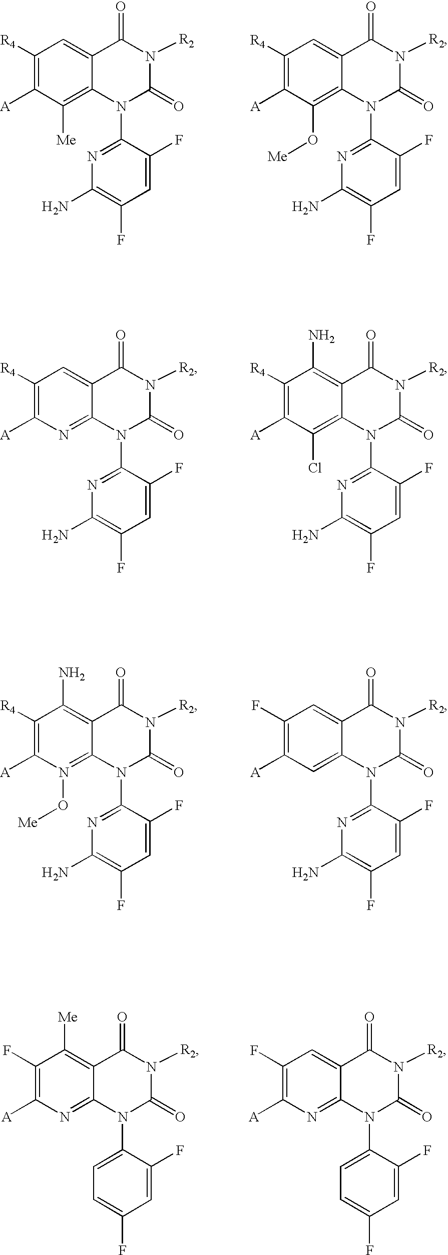

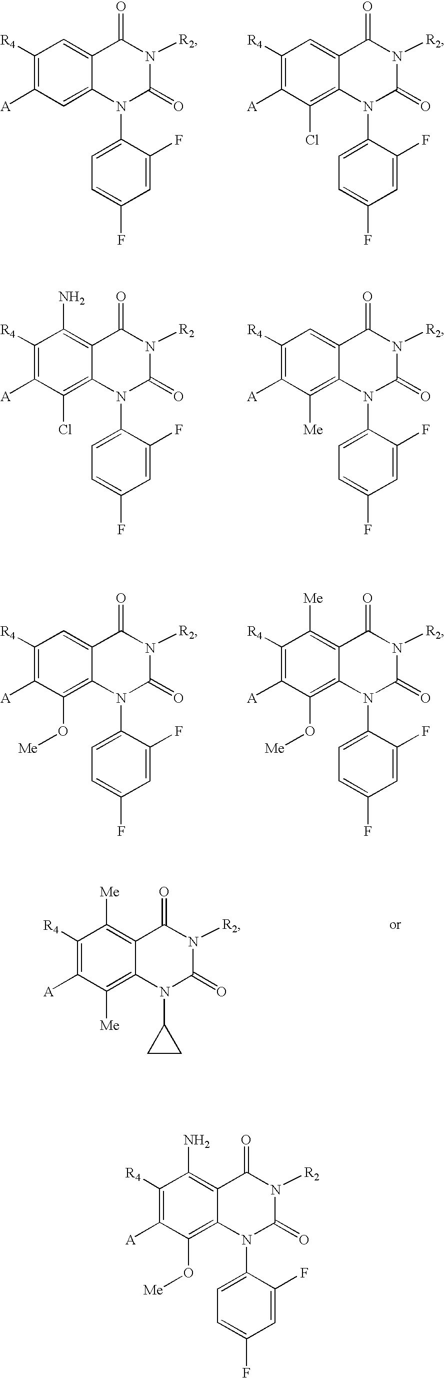

As indicated previously, in compounds of the invention, A is 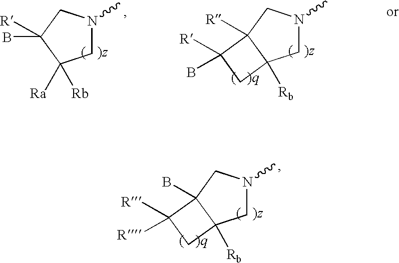
wherein q can be 0, 1, 2, or 3 and z can be 0, 1, or 2. Specifically, z is 0, 1, 2, when q is 2 or 3; alternatively, z is 1 or 2 when q is 0, 1, 2, or 3.

Specifically, Rₐ and R_{b} each independently can be H, (C₁-C₆)alkyl, (C₁-C₆)alkoxy, halo(C₁-C₆)alkyl, halo, or Rₐ and R_{b} taken together with the carbon to which they are attached form C═O, C═NO(C₁-C₆)alkyl, or a 3, 4, 5 or 6-membered substituted or unsubstituted ring. More specifically, Rₐ and R_{b} are each independently H, methyl, ethyl, fluoro, fluoromethyl, trifluoromethyl, fluorethyl, methoxy, MeO—N═, or taken together with the carbons to which they are attached form a cyclopropyl ring.

Specifically, R′, R″, R′″, and R″″ each independently can be H, (C₁-C₆)alkyl, —O(C₁-C₆)alkyl, halo(C₁-C₆)alkyl, aryl, or heteroaryl. More specifically, R′, R″, R′″, and R″″ are each independently H, fluoro, methyl, ethyl, fluoromethyl, fluoroethyl, phenyl, benzyl, or methoxy.

Specifically, B is 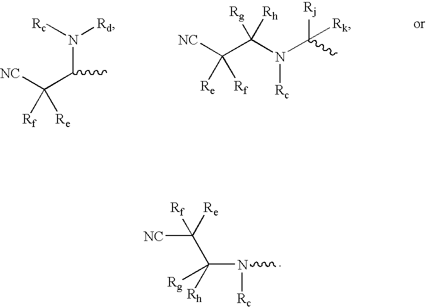
However, when B is 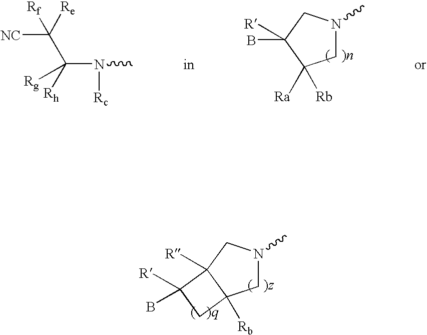
wherein [symbol] indicates the point of attachment, R′ is not —O(C₁-C₆)alkyl.

Specifically, R_{c} and R_{d} each independently can be H, (C₁-C₆)alkylnitrile, 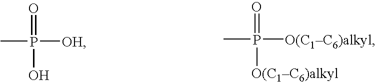
(C₁-C₆)alkyl, (C₃-C₆)cycloalkyl, heteroaryl, SO₂—(C₁-C₆)alkyl, SO₂-aryl, or SO₂-heteroaryl. More specifically, R_{c} and R_{d} each independently are H, methyl, or ethyl.

Specifically Rₑ and R_{f} each independently can be H, C₁-C₆ alkyl, haloalkyl, halo, or Rₑ and R_{f} taken together with the carbon to which they are attached form a 3, 4, 5 or 6-membered substituted or unsubstituted ring. More specifically, Rₑ and R_{f} each independently are H, methyl, or ethyl.

Specifically, R_{g} and Rₕ each independently can be H, C₁-C₆ alkyl, haloalkyl, or taken together with the carbon to which they are attached to form a 3, 4, 5 or 6-membered substituted or unsubstituted ring. More specifically, R_{g} and Rₕ each independently are H, methyl, ethyl, fluoromethyl, difluoromethyl, trifluoroethyl, cyclopropyl, phenyl, isoxazolyl, carboxymethyl, carboxyethyl, or 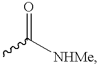
or taken together with the carbons tro which they are attached form

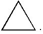

Specifically Rⱼ and Rₖ each independently can be H, (C₁-C₆)alkyl, haloalkyl, (C₁-C₆)alkyl-NR_{c}R_{d}, (C₁-C₆)alkyl-OR_{c}, aryl, heteroaryl, heterocycle, 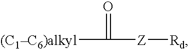
wherein Z is O or NR_{c}, or Rⱼ and Rₖ taken together with the carbon to which they are attached to form a 3, 4, 5 or 6-membered substituted or unsubstituted ring. More specifically, Rⱼ and Rₖ each independently are H, methyl, ethyl, fluoromethyl, difluoromethyl, trifluoroethyl, cyclopropyl, phenyl, isoxazolyl, carboxymethyl, carboxyethyl, or 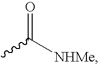
or taken together with the carbons tro which they are attached form

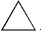

Thus, in compounds of formula I wherein A is 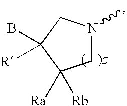
and z is 0, 1, or 2. A is exemplified by any of the following structures: 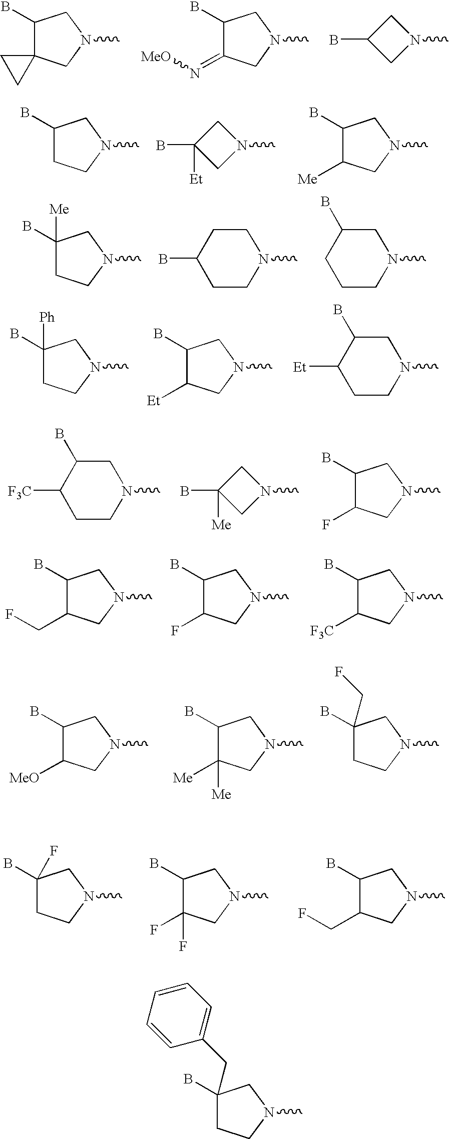
wherein [symbol] indicates the point of attachment and B is 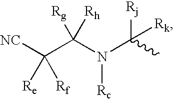
or

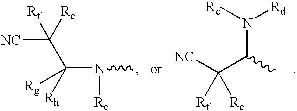

Furthermore in compounds of formula I wherein A is 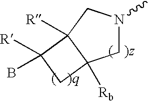
or 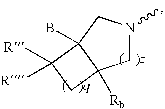
wherein z is 0, 1, or 2 and q is 0, 1, 2, or 3, A is exemplified by any of the following structures: 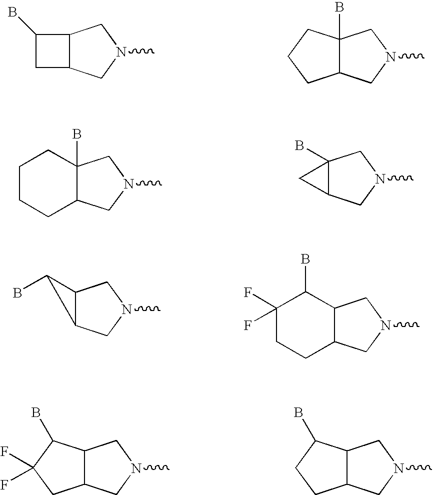
wherein [symbol] indicates the point of attachment and B is 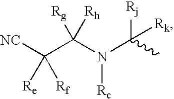
or

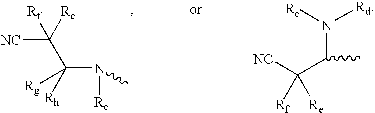

As indicated previously, B can be 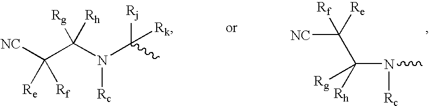
or 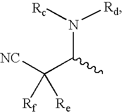
wherein R_{c}, R_{d}, Rₑ, R_{f}, R_{g}, Rₕ, Rⱼ, and Rₖ and have any of the definitions provided above. Thus B can have any of the following structures:

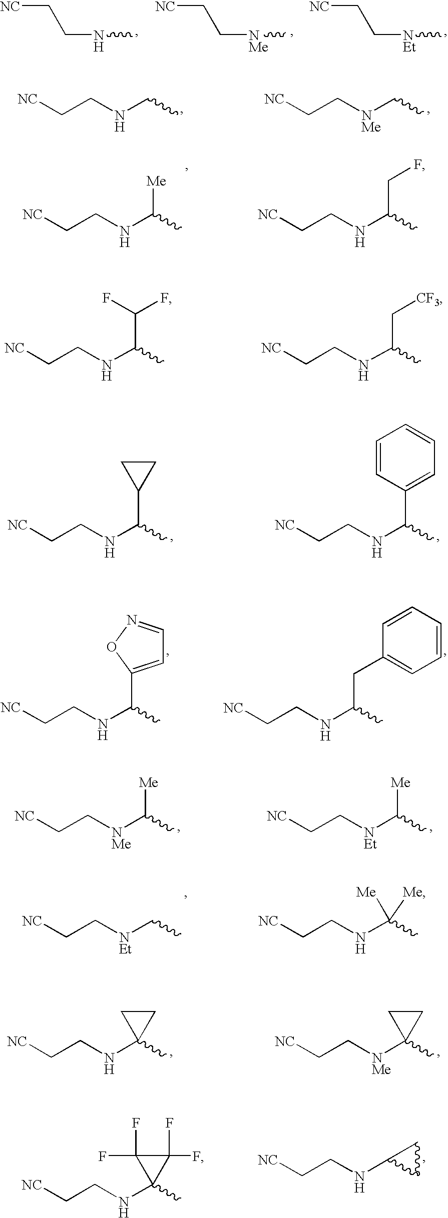

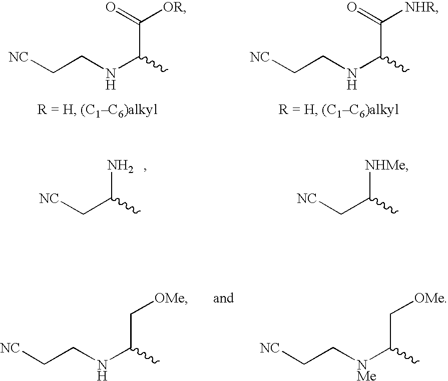

Thus, given the description of A and B, 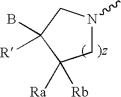
includes any of the following structures: 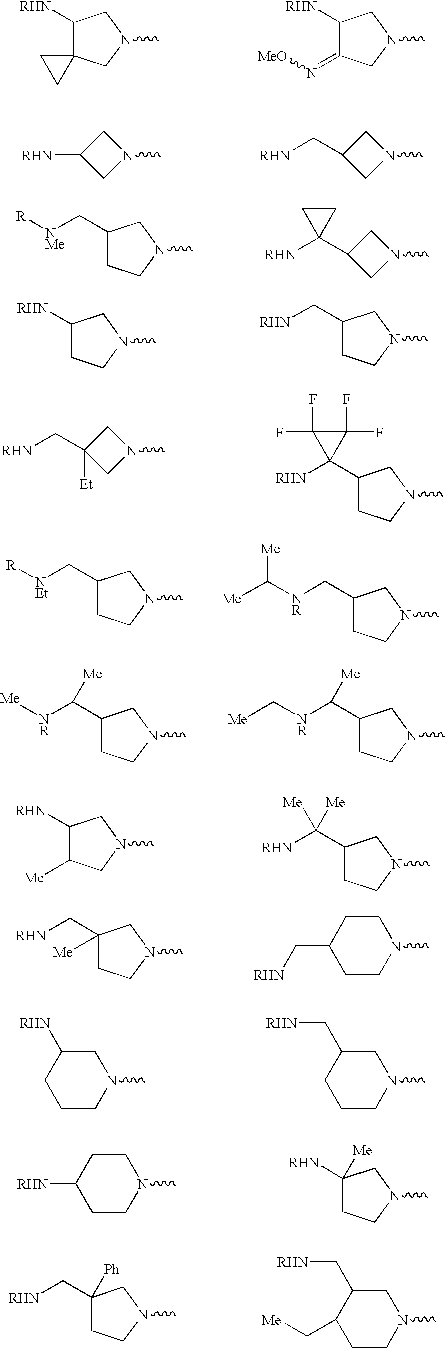 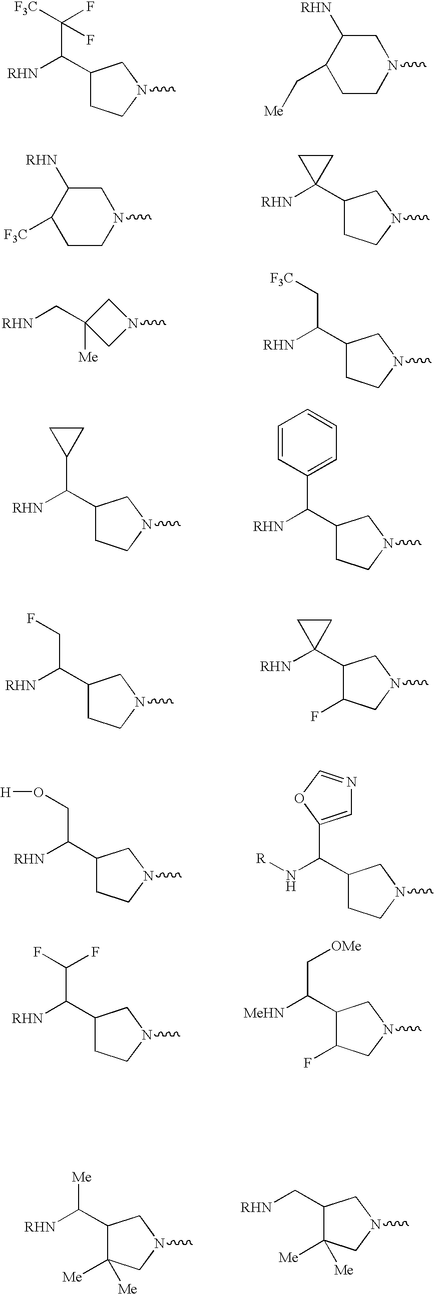 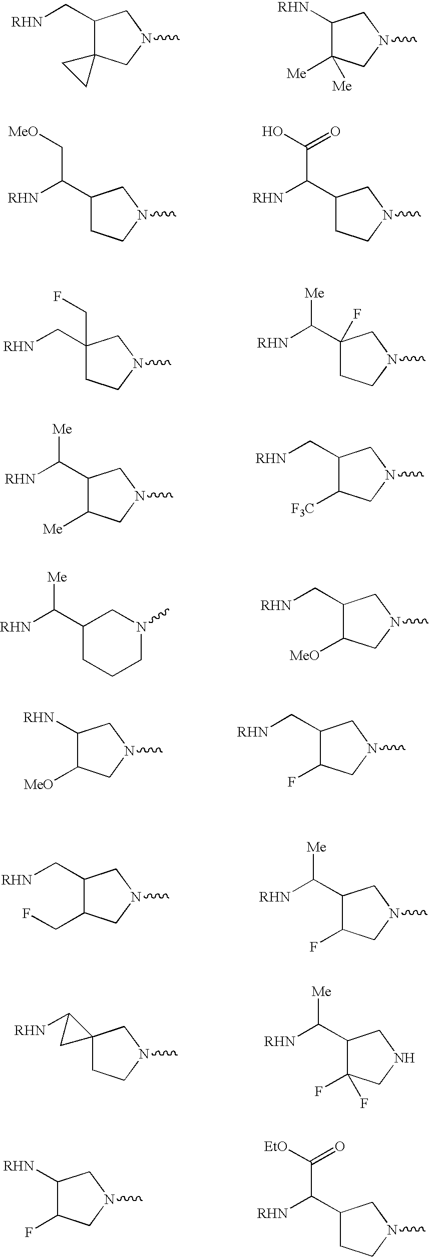 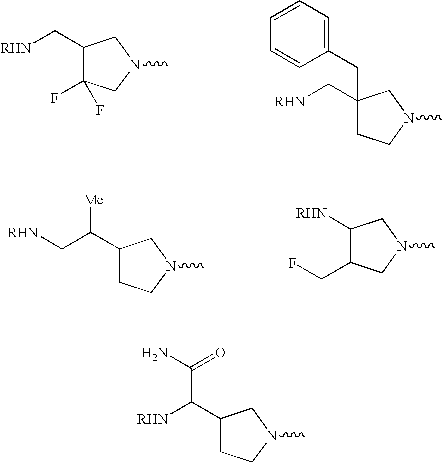
wherein R is CH₂CH₂CN.

Also given the description of A and B, 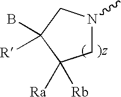
further includes any of the following structures: 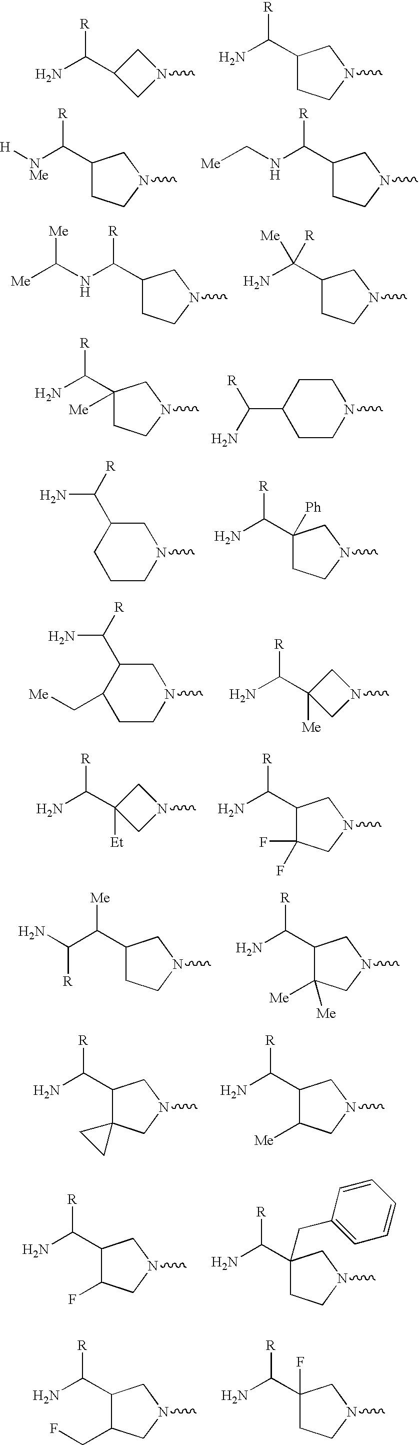 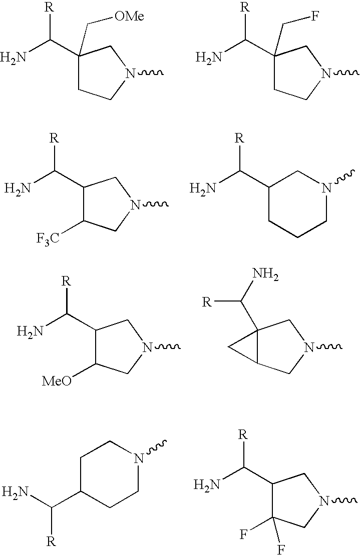
wherein R is CH₂CN.

Also given the description of A and B, 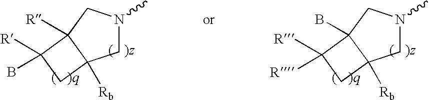
encompasses any of the following structures: 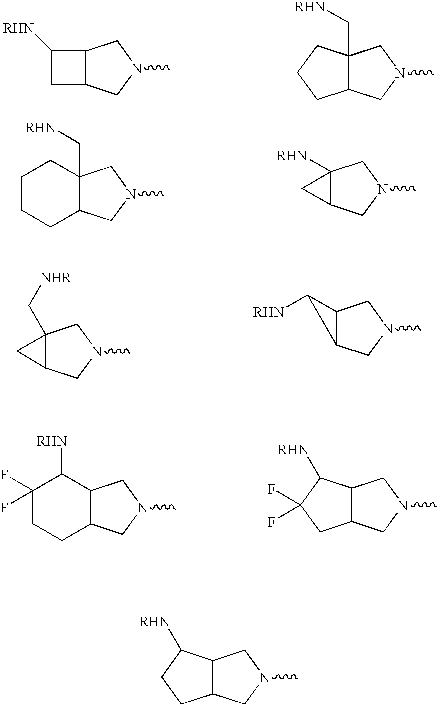
wherein R is CH₂CH₂CN.

Also given the description of A and B, 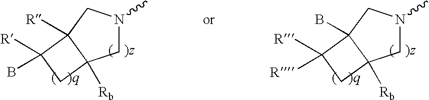
encompasses any of the following structures: 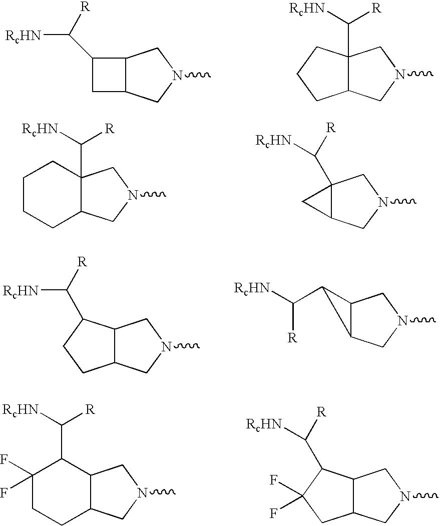
wherein R_{c} is H or (C₁-C₆)alkyl and R is CH₂CN.

We turn now to compounds of formulas II and III, which have the structures:

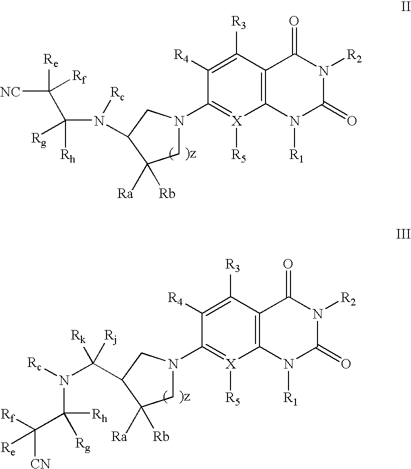

Specific values and embodiments for compounds of formulas II and III are as provided for compounds of formula I with respect to q, z, X, R₁, R₂, R₃, R₄, R₅ and Rₐ, R_{b}, R_{c}, Rₑ, R_{f}, R_{g}, Rₕ, Rⱼ, and Rₖ.

We turn now to a compound of formula IV, which has the structure:

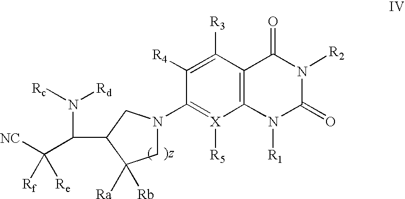

as provided for compounds of formula I with respect to n, X, R₁, R₂, R₃, R₄, R₅ and Rₐ, R_{b}, R_{c}, R_{d}, Rₑ, and R_{f}.

We turn now to a compound of formula V or VI.

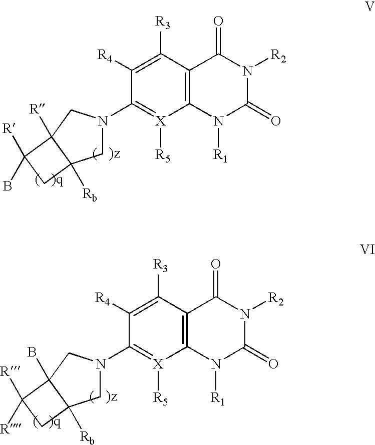

Specific values and embodiments for compounds of formulas V are as provided for compounds of formula I with respect to z, q, X, Rₐ, R₁, R₂, R₃, R₄, R₅ and R′, R″, R′″, R″″, Rₐ, R_{b}, R_{c}, Rₑ, R_{f}, R_{g}, Rₕ, Rⱼ, and Rₖ.

### Preparation of Invention Compounds

Strategies for the preparation of invention compounds are depicted in the following Schemes.

As is readily apparent from this disclosure, compounds of the present invention are characterized by an aminoquinazolinedione core, covalently bound to a C-7 sidechain A-B, wherein A-B can be 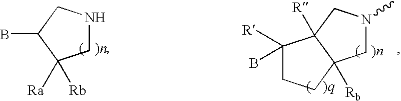
or 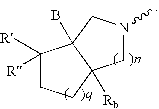
As is retrosynthetically depicted in Scheme I, the invention compounds can be prepared via coupling of a suitably C-7 substituted aminoquinazolinedione core precursor, wherein X is halo, triflate, or a similar reactive group known to the skilled artisan, and an appropriately substituted azetidine, pyrrolidine, piperidine.

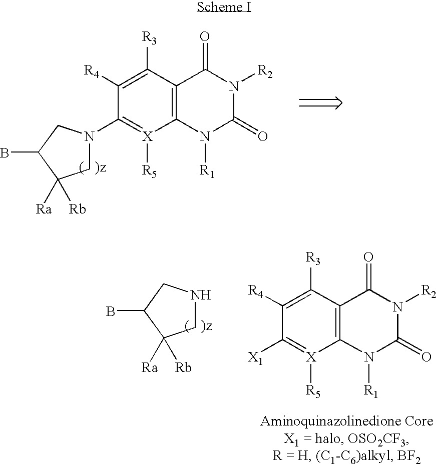

Reflecting the synthetic strategy summarized in Scheme I, the following section describing the preparation of the invention compounds has several parts. The first part describes the synthesis of the requisite aminoquinazolinedione core precursors. The second part describes the synthesis of the requisite C-7 sidechain precursors. The final part describes the coupling of the C-7 sidechain and aminoquinazolinedione core precursors to provide the invention compounds, and details any further chemical elaboration of invention compounds to produce other invention compounds.

A. Synthesis of Aminoquinazolinedione Core Precursors

The quinazolinedione core precursors that are used to prepare the invention compounds can be prepared as described in U.S. patent application Ser. No. 10/182,221, filed Dec. 12, 2001 and references cited therein.

B. Synthesis of C-7 Sidechains and Sidechain Precursors

1. Preparation of 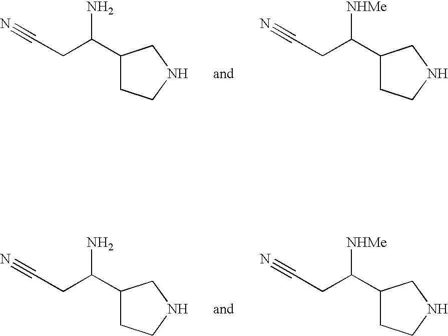
were prepared as provided in Scheme 1. Thus, 3-formyl-pyrrolidine-1-carboxylic acid benzyl ester was allowed to undergo reaction with cyanomethyl-phosphonic acid diethyl ester in the presence of cesium carbonate to provide 3-(2-cyano-vinyl)-pyrrolidine-1-carboxylic acid benzyl ester. Ammonia or methylamine addition to 3-(2-cyano-vinyl)-pyrrolidine-1-carboxylic acid benzyl ester provided the corresponding Michael adduct, which was subsequently tert-butoxycarbonyl (Boc)-protected. Removal of the benzyl ester group under conventional conditions provided the target compound as the protected Boc analogue, which can be submitted to silica gel chromatography to provide each diastereomer and chiral HPLC separation to provide each enantiomer. Purification by chromatography as described herein provides pure samples of 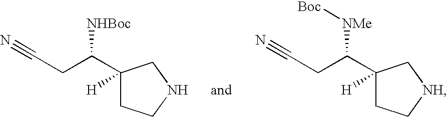
as well as the associated diastereomers, for coupling. Removal of the Boc protecting groups after coupling provides the unprotected amines 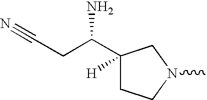
and 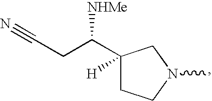
wherein [symbol] indicates a point of attachment.

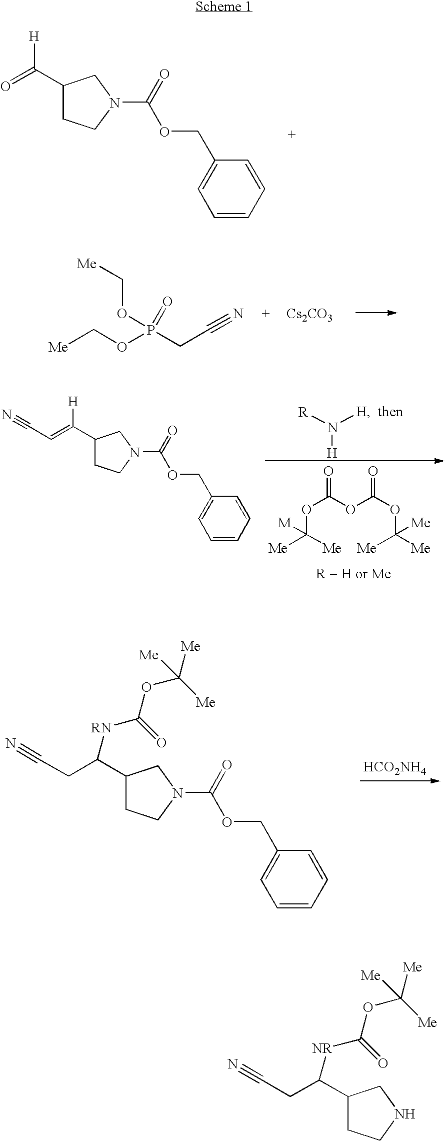

2. Preparation of 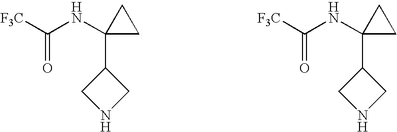
was prepared according to Scheme 2. Thus, 1-benzhydryl-azetidine-3-carbonitrile was converted to 1-(1-benzhydryl-azetidin-3-yl)-cyclopropylamine as provided by Chem. Rev., 1979, Vol. 79, No. 4 and Tet. Lett. 44, 2003, 2485. 1-(1-Benzhydryl-azetidin-3-yl)-cyclopropylamine was converted to N-[1-(1-Benzhydryl-azetidin-3-yl)-cyclopropyl]-2,2,2-trifluoro-acetamide as provided by J. Med. Chem. 1993, vol. 36, No. 7. Hydrogenation of N-[1-(1-benzhydryl-azetidin-3-yl)-cyclopropyl]-2,2,2-trifluoro-acetamide provided the title compound, which can be converted to the free amine and subsequently derivatized after the coupling reaction to the aminoquinazolinedione core.

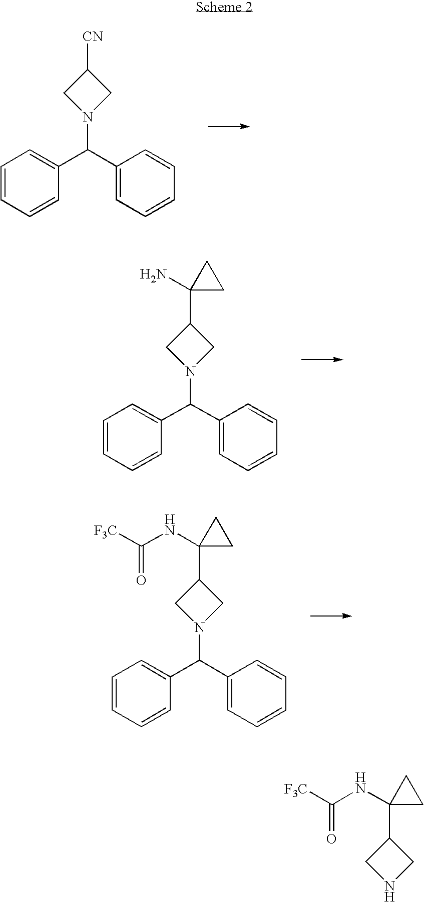

3. Preparation of 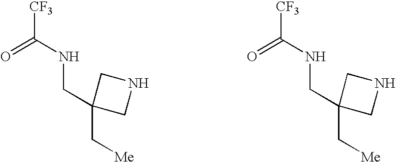
was prepared as provided in Scheme 3. Thus, Grignard reaction of ethylmagnesium bromide with 1-benzhydryl-azetidin-3-one provided the corresponding alcohol, 1-benzhydryl-3-ethyl-azetidin-3-ol. Mesylation of the alcohol moiety in 1-benzhydryl-3-ethyl-azetidin-3-ol under conventional conditions, followed by nucleophilic addition of CN, provided 1-benzhydryl-3-ethyl-azetidine-3-carbonitrile, which was subsequently reduced using lithium aluminum hydride (LAH) to provide C-(1-benzhydryl-3-ethyl-azetidin-3-yl)-methylamine. Protection of C-(1-benzhydryl-3-ethyl-azetidin-3-yl)-methylamine as the trifluoroacetate, followed by hydrogenation, provided the target compound, which can be converted to free amine and subsequently deprotected after the coupling reaction to the aminoquinazolinedione core.

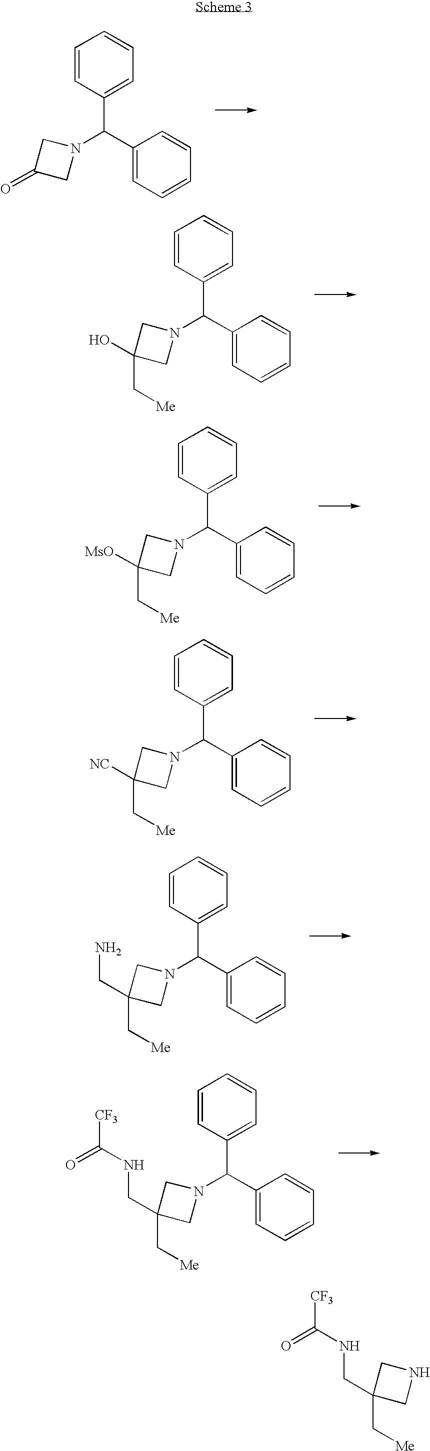

4. Preparation of 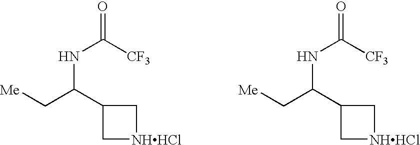
was prepared as provided in Scheme 4. Thus, 1-benzhydryl-azetidine-3-carbonitrile was hydrolyzed under conventional conditions to provide 1-benzhydryl-azetidine-3-carboxylic acid, which was subsequently treated with 1-[3-(dimethylamino)propyl]-3-ethylcarbodiimide hydrochloride and N,O-dimethyl-hydroxyl-amine hydrochloride in the presence of triethylamine to give 1-benzhydryl-azetidine-3-carboxylic acid methoxymethylamide. Grignard addition of ethylmagnesium bromide to 1-benzhydryl-azetidine-3-carboxylic acid methoxymethylamide provided the corresponding ketone, 1-(1-benzhydryl-azetidin-3-yl)-propan-1-one. Reductive amination of 1-(1-benzhydryl-azetidin-3-yl)-propan-1-one using ammonium acetate and sodium cyanoborohydride, followed by treatment with trifluoroacetic anhydride in the presence of an amine base provided 1-(1-benzhydryl-azetidin-3-yl)-propan-1-one. Hydrogenation of 1-(1-benzhydryl-azetidin-3-yl)-propan-1-one gave the target compound, which can be converted to the free amine and subsequently deprotected after the coupling reaction to the aminoquinazolinedione core.

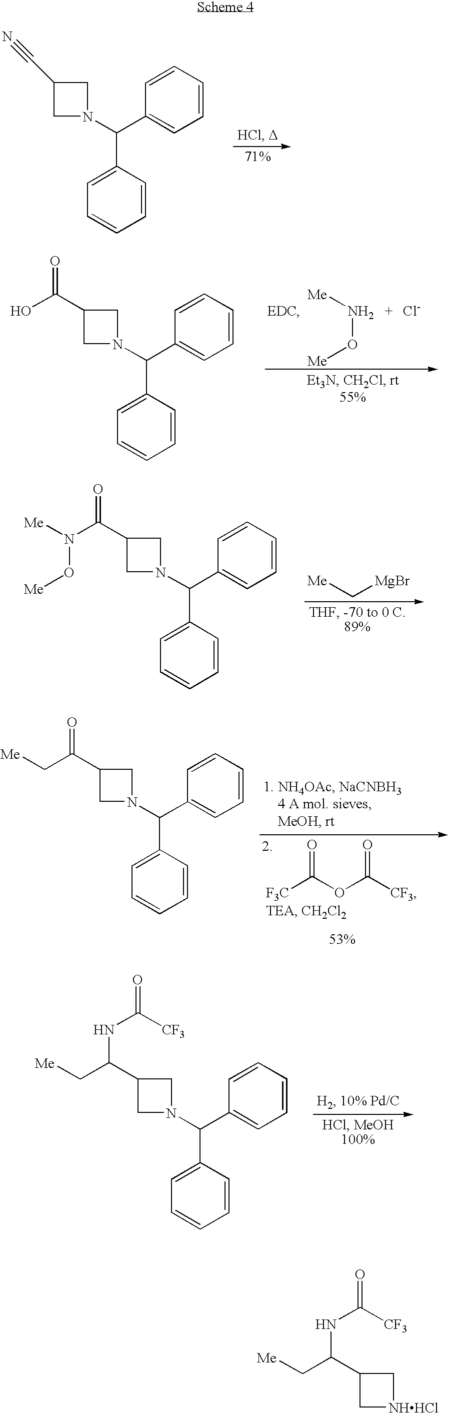

5. Preparation of 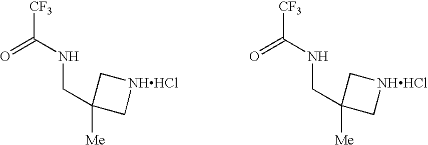
was prepared as provided in Scheme 5. Thus, similar to the synthesis of 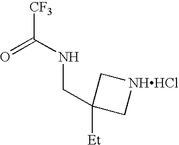
as depicted in Scheme 3, Grignard reaction of methylmagnesium bromide with 1-benzhydryl-azetidin-3-one provided the corresponding alcohol, 1-benzhydryl-3-methyl-azetidin-3-ol. Mesylation of the alcohol moiety in 1-benzhydryl-3-methyl-azetidin-3-ol under conventional conditions, followed by nucleophilic displacement with CN, provided 1-benzhydryl-3-methyl-azetidine-3-carbonitrile, which was subsequently reduced using lithium aluminum hydride (LAH) to provide C-(1-benzhydryl-3-methyl-azetidin-3-yl)-methylamine. Protection of C-(1-benzhydryl-3-methyl-azetidin-3-yl)-methylamine as the trifluoroacetate, followed by hydrogenation, provided the target compound, which can be converted to free amine and subsequently deprotected after the coupling reaction to the aminoquinazolinedione core.

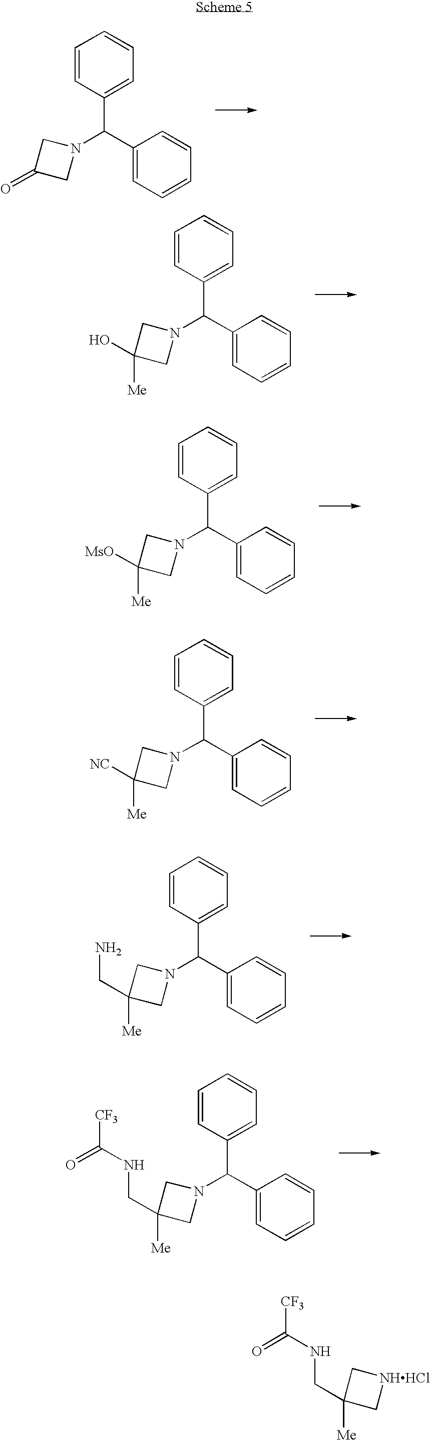

6. Preparation of

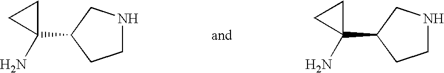

The target compounds 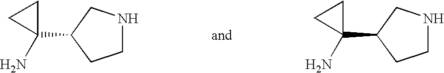
were prepared as provided in Scheme 6. Thus, N-(trimethylsilylmethyl)-α-methylbenzylamine was prepared from the corresponding amine using trimethylsilyl chloride under conventional conditions. Reaction of N-(trimethylsilylmethyl)-α-methylbenzyl amine with formaldehyde in the presence of potassium carbonate and methanol provided N-(methoxymethyl)-N-(trimethylsilylmethyl)-α-methylbenzylamine, which was subsequently converted to 1-(1-phenyl-ethyl)-pyrrolidine-3-carboxylic acid dibenzylamide as a mixture of stereoisomers. Treatment of the amide with ethylmagnesium bromide in the presence or Ti(OiPr)₄ provided the protected target compounds as a separable mixture. The separable diastereomers are deprotected by hydrogenation to give the target compounds.

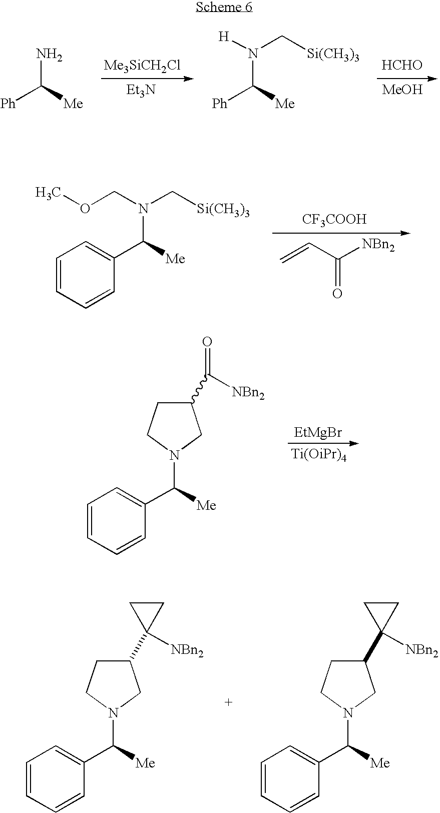

7. Preparation of 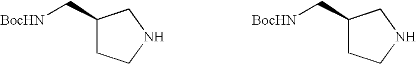
was prepared as provided in Scheme 7. Thus, mesylation of S-1-benzyl-pyrrolidin-3-ol, followed by followed by nucleophilic addition using CN^{-,} provided R-1-benzyl-pyrrolidine-3-carbonitrile. LAH reduction of R-1-benzyl-pyrrolidine-3-carbonitrile provided R—C-(1-Benzyl-pyrrolidin-3-yl)-methylamine. Boc protection of the amine group in R—C-(1-Benzyl-pyrrolidin-3-yl)-methylamine provided (1-benzyl-pyrrolidin-3-ylmethyl)-carbamic acid tert-butyl ester, which was hydrogenated to provide the target compound.

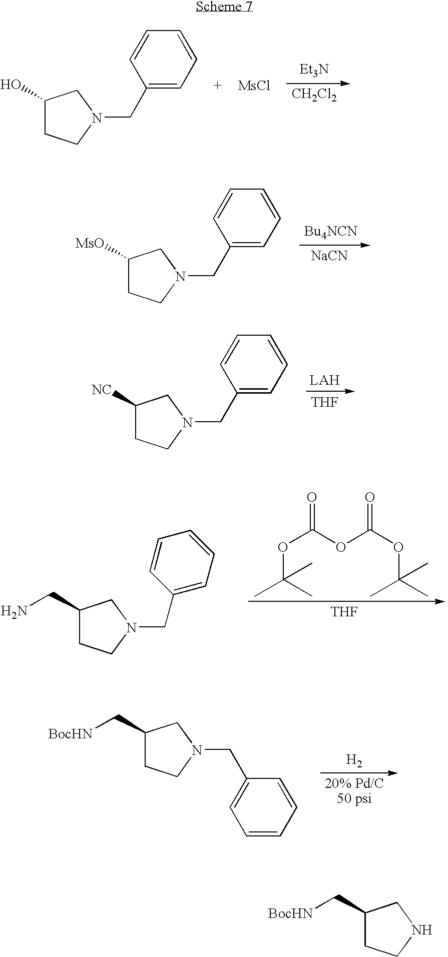

8. Preparation of 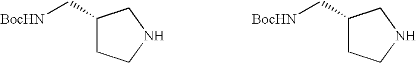
was prepared as provided in Scheme 8. Thus, treatment of 5-oxo-1-(1-phenyl-ethyl)-pyrrolidine-3-carboxylic acid methyl ester with lithium aluminum hydride provided [1-(1-phenyl-ethyl)-pyrrolidin-3-yl]-methanol. Treatment of [1-(1-phenyl-ethyl)-pyrrolidin-3-yl]-methanol with isoindole-1,3-dione in the presence of triphenyl phosphine and diisopropyl azodicarboxylate gave the phthalimide, 2-[1-(1-phenyl-ethyl)-pyrrolidin-3-ylmethyl]-isoindole-1,3-dione. Treatment of the phthalimide with hydrazine hydrate gave C-[1-(1-phenyl-ethyl)-pyrrolidin-3-yl]-methylamine, which was BOC-protected and hydrogenated to provide the target compound, which was converted to free amine and subsequently derivatized after the coupling reaction to the aminoquinazolinedione core.

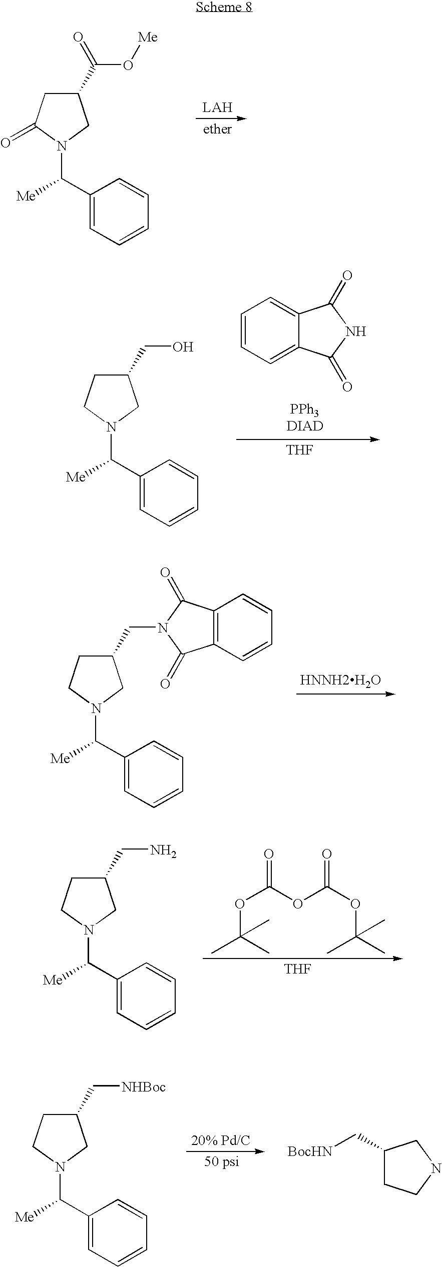

9. Preparation of 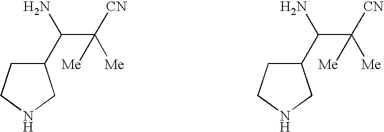
was prepared as provided in Scheme 9. Thus, 1-benzyl-pyrrolidine-3-carboxylic acid ethyl ester was hydrogenated under conventional conditions to provide pyrrolidine-3-carboxylic acid ethyl ester, which was subsequently BOC-protected to provide pyrrolidine-1,3-dicarboxylic acid 1-tert-butyl ester 3-ethyl ester. Reduction of pyrrolidine-1,3-dicarboxylic acid 1-tert-butyl ester 3-ethyl ester provided 3-hydroxymethyl-pyrrolidine-1-carboxylic acid tert-butyl ester, which was oxidized to the corresponding aldehyde 3-formyl-pyrrolidine-1-carboxylic acid tert-butyl ester under Swern-type conditions. 3-(1-tert-butoxycarbonylamino-2-cyano-2,2-dimethyl-ethyl)-pyrrolidine-1-carboxylic acid tert-butyl ester was prepared from 3-formyl-pyrrolidine-1-carboxylic acid tert-butyl ester by the addition of lithiated isopropylcyanide to the intermediate a-amidoalkyl sulfone. Deprotection of 3-(1-tert-butoxycarbonylamino-2-cyano-2,2-dimethyl-ethyl)-pyrrolidine-1-carboxylic acid tert-butyl ester provided the title compound 3-amino-2,2-dimethyl-3-pyrrolidin-3-yl-propionitrile as the dihydrochloride salt. 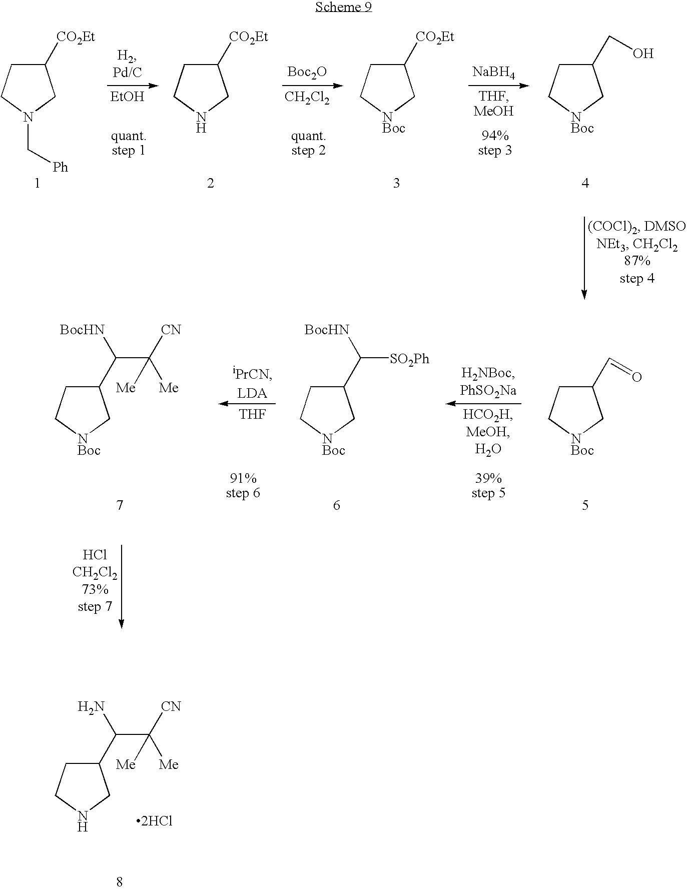
10. Preparation of

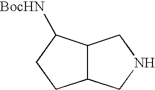

The compound was prepared as provided in WO 96/39407.
11. Preparation of 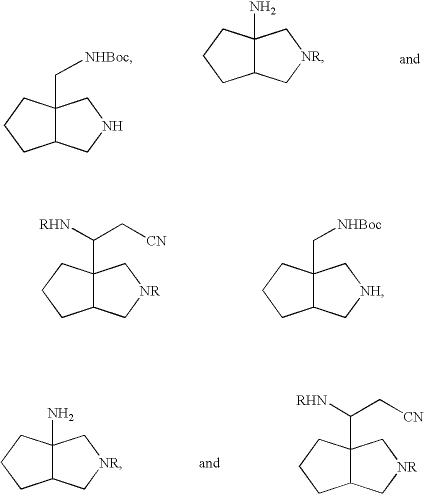
can be prepared as provided in Scheme 11. Thus, [3+2] cycloaddition of cyclopent-1-enecarboxylic acid methyl ester or the like with benzyl-methoxymethyl-trimethylsilanylmethyl-amine under conditions readily available to the skilled artisan and discussed herein provides 2-benzyl-hexahydro-cyclopenta[c]pyrrole-3a-carboxylic acid methyl ester.

Preparation of 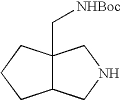
commences with hydride reduction of 2-benzyl-hexahydro-cyclopenta[c]pyrrole-3a-carboxylic acid methyl ester using an aluminum hydride or borohydride to provide (2-benzyl-hexahydro-cyclopenta[c]pyrrol-3a-yl)-methanol. Conversion of the alcohol moiety in (2-benzyl-hexahydro-cyclopenta[c]pyrrol-3a-yl)-methanol to a leaving group such as the mesylate or tosylate, followed by displacement with a primary or secondary amine, and a protection deprotection sequence, provides (2-benzyl-hexahydro-cyclopenta[c]pyrrol-3a-ylmethyl)-carbamic acid tert-butyl ester, 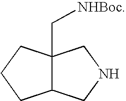
Alternatively, if the reduction of the ester moiety in 2-benzyl-hexahydro-cyclopenta[c]pyrrole-3a-carboxylic acid methyl ester is stopped at the aldehyde oxidation state (for example, by employing DIBALH as the reducing agent), a reductive amination using ammonium formate or a primary alkyl amine such as methyl or ethyl amine can be employed to provide the aminated product. Reductive amination conditions and reagents are readily known to the skilled artisan.

Preparation of 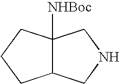
commences with saponification of the ester moiety in 2-benzyl-hexahydro-cyclopenta[c]pyrrole-3a-carboxylic acid methyl ester top provide 2-benzyl-hexahydro-cyclopenta[c]pyrrole-3a-carboxylic acid. Curtius rearrangement of 2-benzyl-hexahydro-cyclopenta[c]pyrrole-3a-carboxylic acid using conditions and rearrangements readily available to the skilled artisan provides 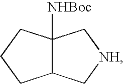
(hexahydro-cyclopenta[c]pyrrol-3a-yl)-carbamic acid tert-butyl ester.

Preparation of 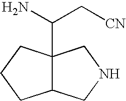
commences with DIBALH reduction of 2-benzyl-hexahydro-cyclopenta[c]pyrrole-3a-carboxylic acid methyl ester to provide the corresponding aldehyde. Methyleneation of the aldehyde under Wittig- or Horner-Wadsworth-Emmons-type conditions provides 3-(2-benzyl-hexahydro-cyclopenta[c]pyrrol-3a-yl)-acrylonitrile. Michael addition of ammonia or a primary alkyl amine to 3-(2-benzyl-hexahydro-cyclopenta[c]pyrrol-3a-yl)-acrylonitrile provides [2-Cyano-1-(hexahydro-cyclopenta[c]pyrrol-3a-yl)-ethyl]-carbamic acid tert-butyl ester. 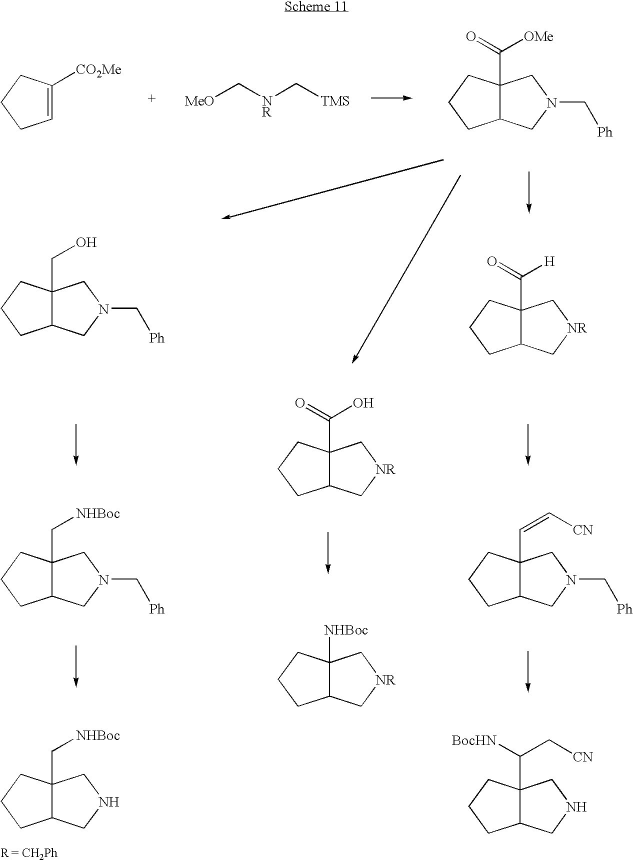
C. Coupling of C-7 Sidechain and Aminoquinazolinedione Core Precursors to Provide Invention Compounds and Invention Compound Precursors

Coupling of the sidechain precursor to the quinazolinedione core precursor to provide the compounds of the present invention occurs as described in WO/02 102793, priority date Jun. 19, 2001 and WO/01 53273, priority date Oct. 18, 2000, and references cited therein.

D. Post-Coupling Transformations

Coupling of the sidechain precursors to the aminoquinazolinedione core precursors may give rise directly to invention compounds. Alternatively, post-coupling transformations may be necessary to give rise to invention compounds. Typical post-coupling transformation include deprotection of protected amines to provide invention compounds of formula II, as depicted in Scheme III. Deprotection, as well as reaction with acrylonitrile or the like give rise to invention compounds of formulas III and IV.

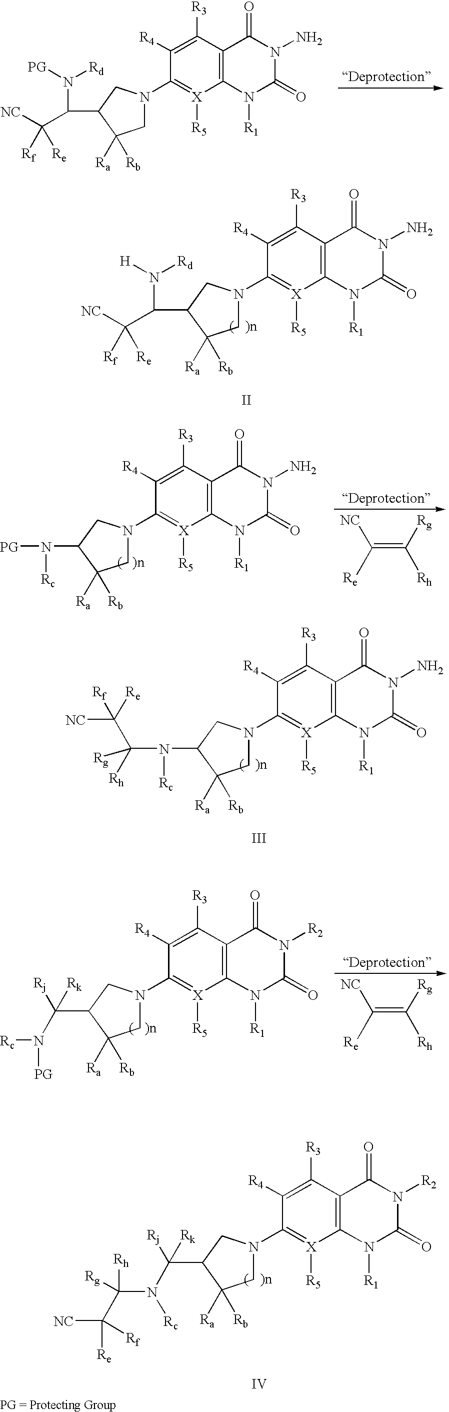

### Pharmaceutical Formulations

The present invention also provides pharmaceutical compositions which comprise a bioactive invention compound or a salt such or a pharmaceutically acceptable salt thereof and optionally a pharmaceutically acceptable carrier. The compositions include those in a form adapted for oral, topical or parenteral use and can be used for the treatment of bacterial infection in mammals including humans.

The compounds, such as antibiotic compounds, also referred to herein as antimicrobial compounds, according to the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

### Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing a bacterial infection in a subject, such as a human or other animal subject, comprising administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985; 28:766; Heifetz, et. al., Antimicrob., 1974; 6:124). The results of the evaluation are shown in Tables 1A and B.

TABLE 1A

| | Minimum Inhibitory Concentrations | | |
|---|---|---|---|
| | μg/mL Gram Negative Bacteria | | |
| | H. influenzae | M. catarrhalis | E. coli |
| | HI-3542 | BC-3531 | 2026 |
| Compound | | | |
| 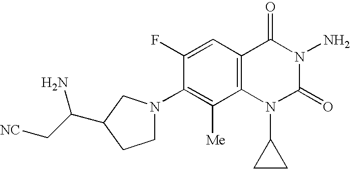 stereoisomeric mixture | 2 | 0.5 | 8 |
| 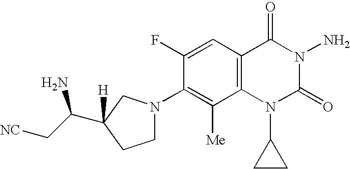 racemic mixture of enantiomers | 4 | 16 | 32 |
| 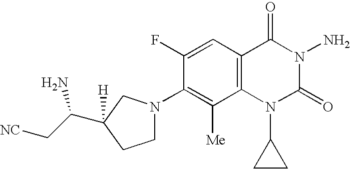 single enantiomer | 1 | 2 | 8 |
| 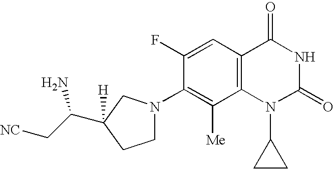 single enantiomer | 4 | 4 | 32 |
| 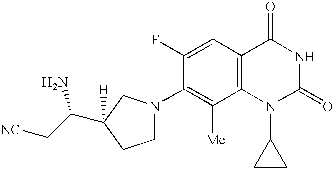 single enantiomer | 2 | 4 | 8 |
| 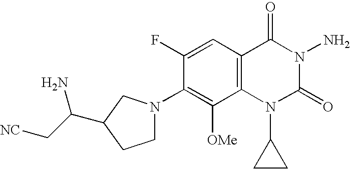 stereoisomeric mixture | 8 | 16 | 64 |
| 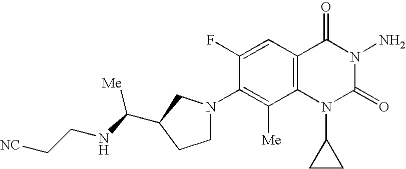 single enantiomer | 0.5 | 1 | 4 |
| 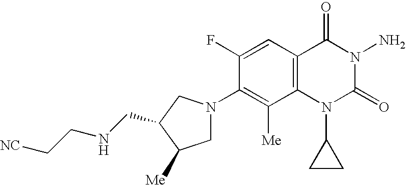 racemic mixture of enantiomers | 2 | 4 | 8 |
| 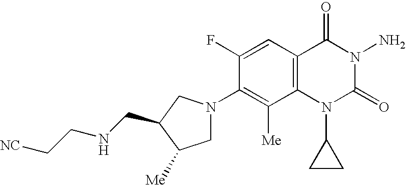 racemic mixture of enantiomers | 4 | 4 | 8 |
| 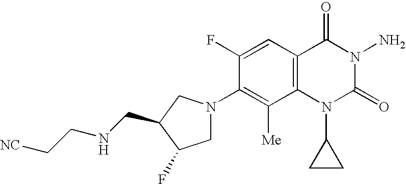 racemic mixture of enantiomers | 8 | 16 | 32 |
| 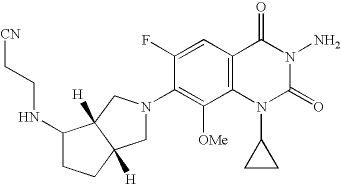 stereoisomeric mixture | 8 | 64 | 64 |
| 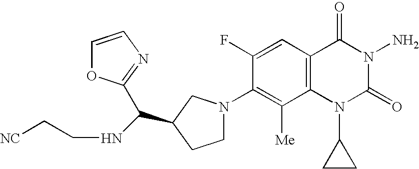 stereoisomeric mixture | 4 | 16 | 16 |
| 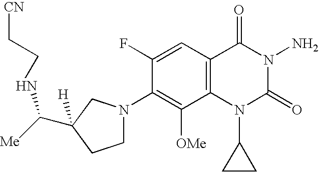 single enantiomer | 16 | 16 | 64 |
| 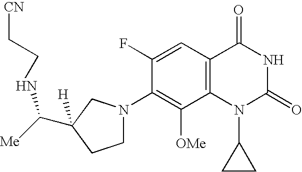 single enantiomer | 64 | 64 | 64 |
| Compound No. or | | | |
| Example No. | | | |
| 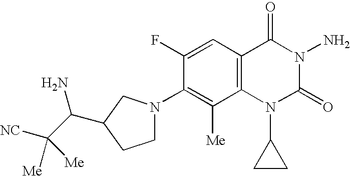 stereoisomeric mixture | 32 | 5.25 | 64 |
| 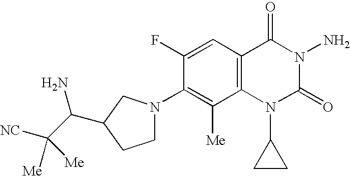 stereoisomeric mixture | 32 | 16 | 64 |
| 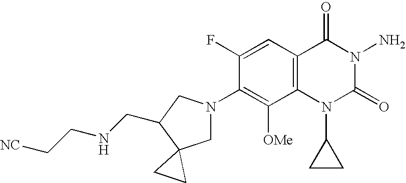 stereoisomeric mixture | 1 | 8 | 2 |

NT = Not tested

TABLE 1B

| | Minimum Inhibitory Concentrations | | | |
|---|---|---|---|---|
| | μg/mL Gram Positive Bacteria | | | |
| | E. faecalis | S. pneumo | S. aureus | S pyogenes |
| | MGH-2 | SV-1 | UC-76 | C203 |
| Compound Structure or | | | | |
| Example No. | | | | |
| 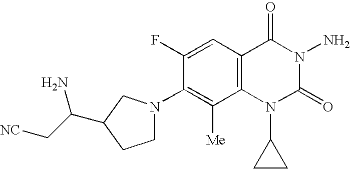 stereoisomeric mixture | 0.5 | 0.125 | 0.25 | 0.125 |
| 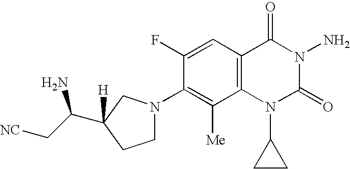 racemic mixture of enantiomers | 4 | 0.5 | 1 | 0.5 |
| 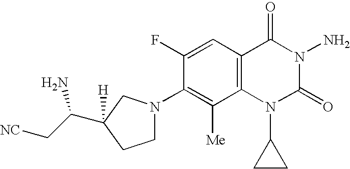 single enantiomer | 0.25 | 0.06 | 0.125 | 0.03 |
| 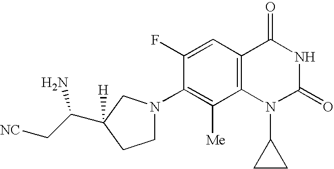 single enantiomer | 0.5 | 0.125 | 0.25 | 0.125 |
| 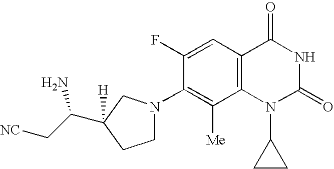 single enantiomer | 1 | 0.125 | 0.5 | 0.25 |
| 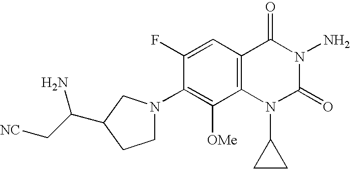 stereoisomeric mixture | 2 | 0.5 | 2 | 0.5 |
| 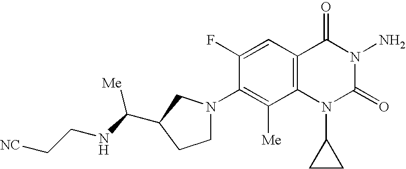 single enantiomer | 0.125 | 0.015 | 0.06 | 0.03 |
| Compound Structure | | | | |
| 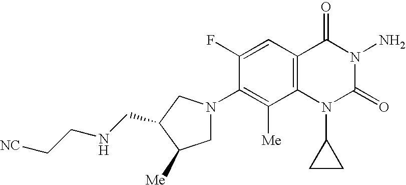 single enantiomer | 0.5 | 0.06 | 0.25 | 0.06 |
| 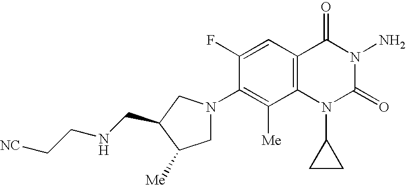 single enantiomer | 0.5 | 0.125 | 0.25 | 0.125 |
| 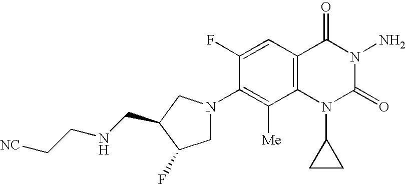 racemic mixture of enantiomers | 4 | 1 | 2 | 1 |
| 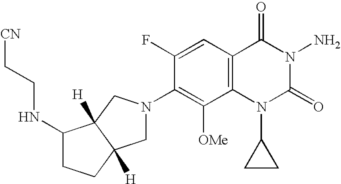 stereoisomeric mixture | 4 | 0.5 | 1 | 1 |
| 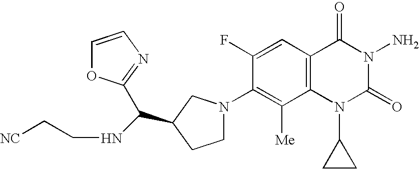 stereoisomeric mixture | 32 | 8 | 8 | 8 |
| 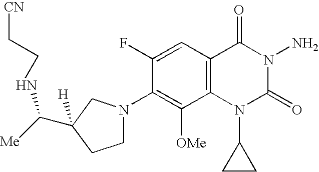 single enantiomer | 1 | 0.125 | 0.25 | 0.125 |
| 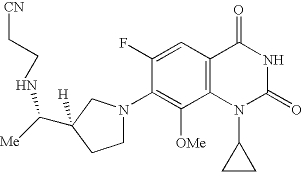 single enantiomer | 2 | 0.25 | 1 | 0.5 |
| 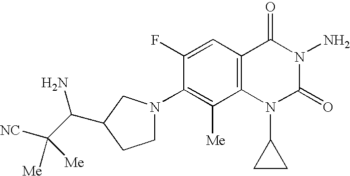 stereoisomeric mixture | 32 | 32 | 12 | 32 |
| 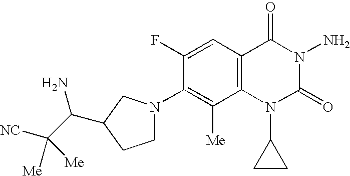 stereoisomeric mixture | 16 | 8 | 8 | 16 |
| 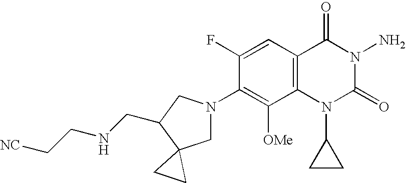 stereoisomeric mixture | 1 | 0.06 | 0.25 | 0.06 |

The following examples are provided to illustrate but not limit the claimed invention.

A. Synthesis of Sidechain Precursors

### EXAMPLE 1

### Preparation of (2-Cyano-1-pyrrolidin-3-yl-ethyl)-carbamic acid tert-butyl ester

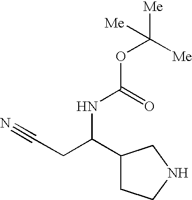

### A. 3-(2-Cyano-vinyl)-pyrrolidine-1-carboxylic acid benzyl ester

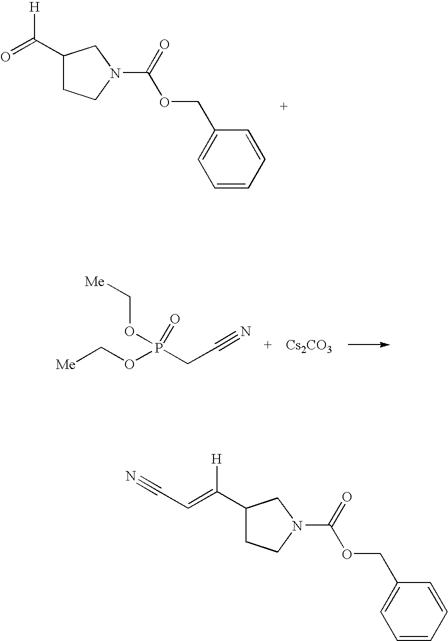

A solution of 3-formyl-pyrrolidine-1-carboxylic acid benzyl ester (2.25 g, 9.65 mmol), cyanomethyl-phosphonic acid diethyl ester (1.88 g, 10.6 mmol) and cesium carbonate (3.46 g, 10.6 mmol) in dry THF (100 mL) was heated at 50° C. for 3 hours. The solvent was removed in vacuo. The crude residue was taken up in ethyl acetate (100 mL) and washed with saturated NH₄Cl (100 mL), brine (100 mL), dried with MgSO₄ and concentrated in vacuo. The crude residue was purified on a 40 g silica gel column (0 to 60% ethyl acetate in hexanes) to give 2.22 g of the title compound as a mixture of E and Z isomers (yield: 90%). MS (APCI+): m/z 257 (M+H)⁺.
B. 3-(1-tert-Butoxycarbonylamino-2-cyano-ethyl)-pyrrolidine-1-carboxylic acid benzyl ester

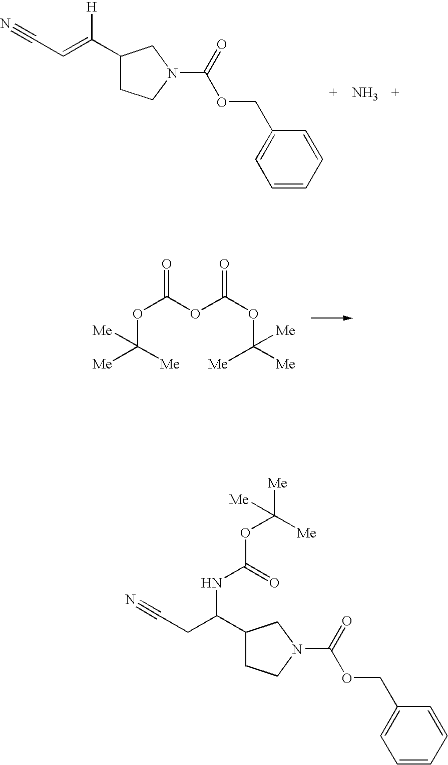

To a solution of 3-(2-cyano-vinyl)-pyrrolidine-1-carboxylic acid benzyl ester (8.24 g, 32.1 mmol) in absolute ethanol (100 mL) was added ammonia (ca. 5 mL) and the solution was heated in a sealed reactor at 80-100° C. for 3 days. The solution was concentrated in vacuo. The resulting amine was dissolved in THF (100 mL), Boc anhydride (8.76 g, 40.2 mmol) was added, and the solution was stirred at room temperature for 18 hours. The solution was concentrated in vacuo. The residue was taken up in ethyl acetate (100 mL), washed with saturated aqueous NH₄Cl (100 mL) and brine (100 mL), dried with MgSO₄ and concentrated in vacuo. The crude product was purified on a 330 g silica gel column (10 to 50% ethyl acetate in hexanes) to give 9.28 g of the title compound as a 1:1 mixture of diastereomers (yield: 77%). MS (APCI+): m/z 274 (M+H-Boc)⁺.

### C. (2-Cyano-1-pyrrolidin-3-yl-ethyl)-carbamic acid tert-butyl ester

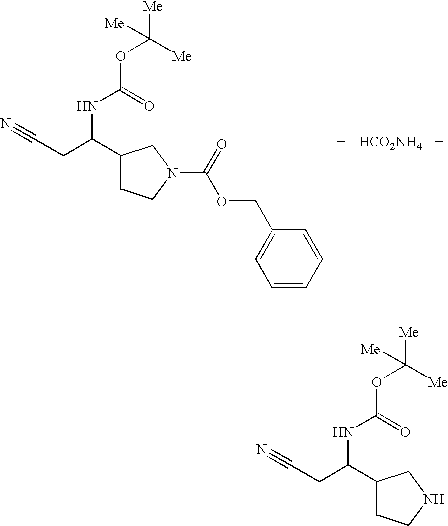

A solution of 3-(1-tert-butoxycarbonylamino-2-cyano-ethyl)-pyrrolidine-1-carboxylic acid benzyl ester (2.00 g, 5.36 mmol) and ammonium formate (1.00 g, 16.1 mmol) in methanol (50 mL) was purged with nitrogen and then 10% Pd/C (0.5 g) was added. The mixture was stoppered and stirred at room temperature for 17 hours. The solution was filtered through Celite and solids were rinsed with methanol. The filtrate was concentrated in vacuo to give 1.25 g of the title compound (yield: 97%). MS (APCI+): m/z 240 (M+H)⁺.

### EXAMPLE 2

### Preparation of N-(1-Azetidin-3-yl-cyclopropyl)-2,2,2-trifluoro-acetamide

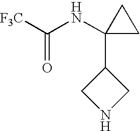

### A. 1-(1-Benzhydryl-azetidin-3-yl)-cyclopropylamine

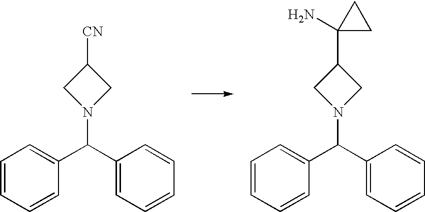

See Chem. Rev., 1979, Vol. 79, No. 4; Tet. Lett. 44, 2003, 2485

To a solution of 1-benzhydryl-azetidine-3-carbonitrile (10 g) in THF (200 mL) were added successively at room temperature titanium isopropoxide (Ti(OiPr)₄) (1 equivalent) and ethylmagnesium bromide (2.2 equivalents). The resulting reaction mixture was stirred for 30 minutes. Boron trifluoride diethyl etherate (BF₃OEt₂)(2 equivalents) was then added. Stirring was continued for a period of 30 minutes. A solution of 10% sodium hydroxide was added, and the mixture was extracted three times with ethyl acetate (EtOAc). The combined ethyl acetate layers were dried over Na₂SO₄, and concentrated. The crude material was purified by chromatography (EtOAc to 7:3 EtOAc:EtOH) to yield the title compound as a yellow solid (4.96 g, 44% yield). MS (APCI+): m/z 279 (M+H)⁺.

### B. N-[1-(1-Benzhydryl-azetidin-3-yl)-cyclopropyl]-2,2,2-trifluoro-acetamide

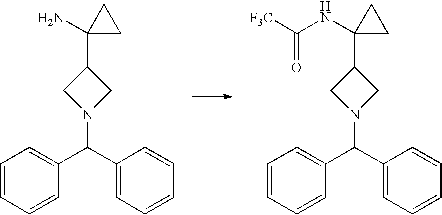

See J. Med Chem. 1993, Vol. 36, No. 7

To a stirred solution of 1-(1-benzhydryl-azetidin-3-yl)-cyclopropylamine (2.5 g) in chloroform (60 mL) was added a solution of trifluoroacetic anhydride (1.25 equivalents) in chloroform (30 mL) dropwise at room temperature. The reaction was stirred for two hours, then washed with 10% NaHCO₃, and subsequently brine. The solution was then concentrated and purified by chromatography (gradient: 3:1 hexanes:EtOAc to EtOAc) yielding 0.57 g (17% yield) of the title compound. MS(APCI+): m/z 375 (M+H)⁺.

### C. N-(1-Azetidin-3-yl-cyclopropyl)-2,2,2-trifluoro-acetamide

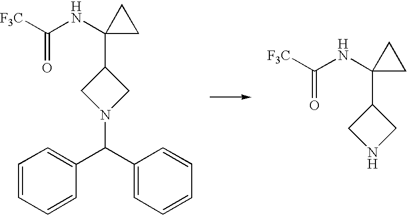

To N-[1-(1-benzhydryl-azetidin-3-yl)-cyclopropyl]-2,2,2-trifluoro-acetamide in methanol was added 10% Pd/C (20%) and hydrochloric acid (1 equivalent). The resulting mixture was stirred under an atmosphere of hydrogen gas overnight. The mixture was then filtered through a pad of celite and the filtrate was concentrated to yield a mixture of the azetidinium hydrochloride and diphenylmethane (0.56 g, 90% yield). The crude mixture was taken on to the next reaction without purification, MS(APCI+): m/z 209 (M+H)⁺.

### EXAMPLE 3

### Preparation of N-(3-Ethyl-azetidin-3-ylmethyl)-2,2,2-trifluoro-acetamide

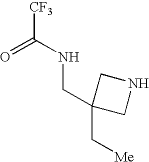

### A. 1-Benzhydryl-3-ethyl-azetidin-3-ol

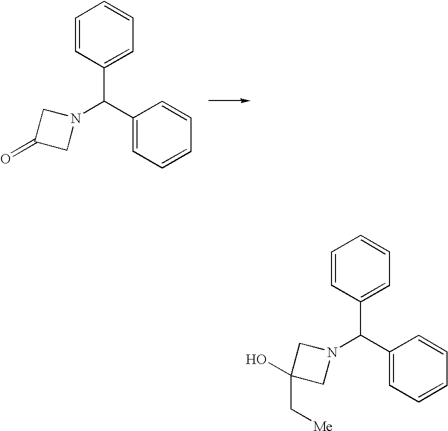

To a solution of 1-benzhydryl-azetidin-3-one (10 g) in diethylether (200 mL) cooled to 0° C. with an ice bath was added dropwise a solution of ethylmagnesium bromide in ether (3.0 M, 2 equivalents). The reaction was allowed to stir at 0° C. until the bath warmed and then reacted at room temperature for three days. The reaction was quenched with aqueous ammonium chloride and then extracted three times with EtOAc. The organic extract washed with brine, dried, and then concentrated. The product was purified by flash chromatography (2:1 hexanes:EtOAc) to give the title compound (6.33 g, 56%), MS (APCI+): m/z 268 (M+H)⁺.

### B. Methanesulfonic acid 1-benzhydryl-3-ethyl-azetidin-3-yl ester

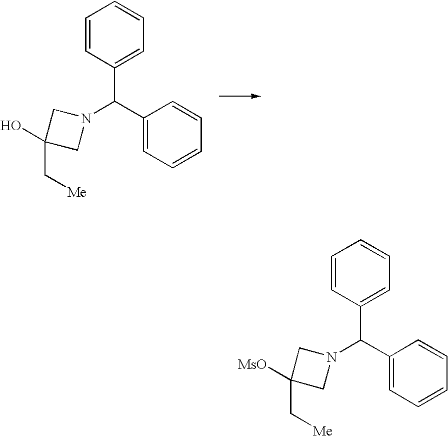

To a cooled (0° C.) solution of the 1-benzhydryl-3-ethyl-azetidin-3-ol (6.33 g) and triethylamine (1.3 equivalents) in dichloromethane (100 mL) was added a solution of methanesulfonyl chloride (1.3 equivalents) in dichloromethane (30 mL) dropwise. As soon as all of the methanesulfonyl chloride had been added, the cooling bath was removed, and the reaction was allowed to stir at room temperature for 1 hour. The solution was then diluted with more dichloromethane and washed with water two times. The organic solution was then dried and concentrated (8.01 g, 98% yield). The crude material was used in the next step without further purification.

### C. 1-Benzhydryl-3-ethyl-azetidine-3-carbonitrile

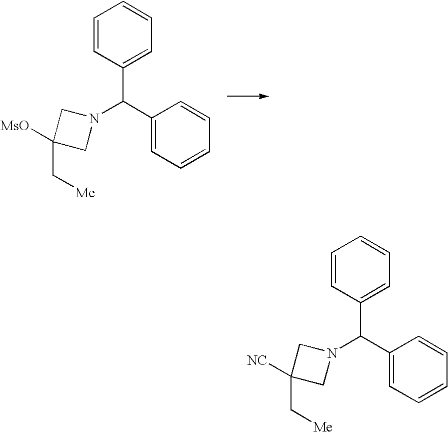

To a solution of methanesulfonic acid 1-benzhydryl-3-ethyl-azetidin-3-yl ester (8.01 g) in dimethylformamide (DMP) (120 mL) at room temperature was added sodium cyanide (2.5 equivalents) in water (40 mL) dropwise. The solution was then heated to 60° C. and stirred overnight The solution was then diluted with 500 mL water and the precipitate was extracted into EtOAc 3 times. The organic extract washed with water 2 times and then dried over Na₂SO₄ and concentrated in vacuo. The product was purified by chromatography (gradient: 9:1 hex:EtOAc to EtOAc) to give the title compound (5.50 g, 86% yield), MS (APCI+): m/z 277 (M+H)⁺.

### D. C-(1-Benzhydryl-3-ethyl-azetidin-3-yl)-methylamine

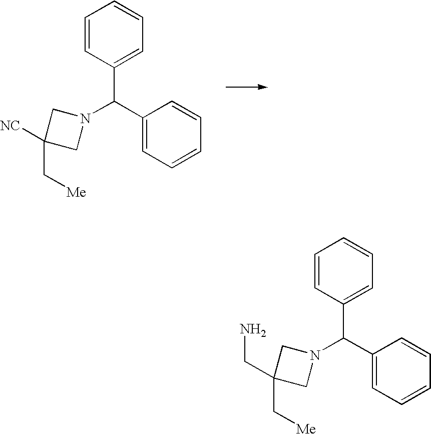

To a solution of 1-benzhydryl-3-ethyl-azetidine-3-carbonitrile (5.50 g) in THF (60 mL) was added LAH (3.5 equivalents) in THF (1 M) slowly. The solution was refluxed for 2 hours. The reaction was then cooled to room temperature and 100 mL diethylether was added followed by 2.8 mL water then 2.8 mL 10% NaOH then 5.6 mL water. After 30 minutes of vigorous stirring the mixture was filtered. The aluminum salts were washed 5 times with THF. The combined organic filtrates were dried, and concentrated. The crude product was used in the next step without further purification. 5.16 g, 92% yield, MS (APCI+): m/z 281 (M+H)⁺.

### E. N-(1-Benzhydryl-3-ethyl-azetidin-3-ylmethyl)-2,2,2-trifluoro-acetamide

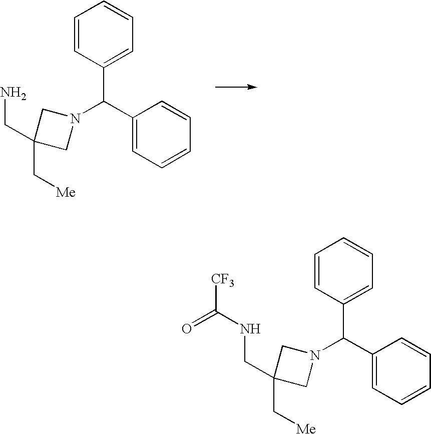

To a stirred solution of C-(1-benzhydryl-3-ethyl-azetidin-3-yl)-methylamine (5.16 g) in chloroform (120 mL) was added a solution of trifluoroacetic anhydride (1.25 equivalents) in chloroform (60 mL) dropwise at room temperature. The reaction was stirred for two hours, then washed with 10% NaHCO₃, then brine. The solution was then dried, then concentrated and purified by chromatography (3:1 hexanes:EtOAc to EtOAc) to provide the title compound (3.67 g, 53% yield), MS (APCI+): m/z 377.3 (M+H)⁺.

### F. N-(3-Ethyl-azetidin-3-ylmethyl)-2,2,2-trifluoro-acetamide

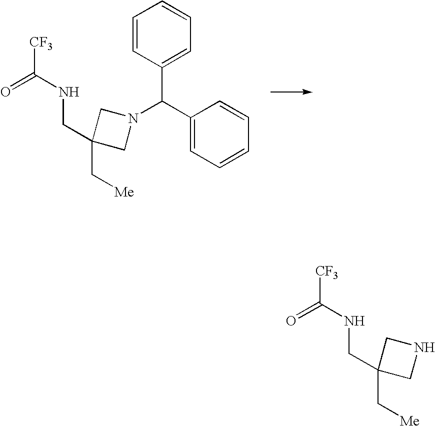

N-(1-Benzhydryl-3-ethyl-azetidin-3-ylmethyl)-2,2,2-trifluoro-acetamide (3.67 g) was hydrogenated (Pd/C in 100 mL MeOH) with one equivalent of HCl overnight to give 2.40 g, (100% yield) of the title compound which was used without purification. MS (APCI+): m/z 211 (M+H)⁺.

### EXAMPLE 4

### Preparation of N-(1-Azetidin-3-yl-propyl)-2,2,2-trifluoroacetamide hydrochloride

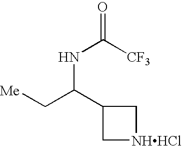

### A. 1-Benzhydryl-azetidine-3-carboxylic acid

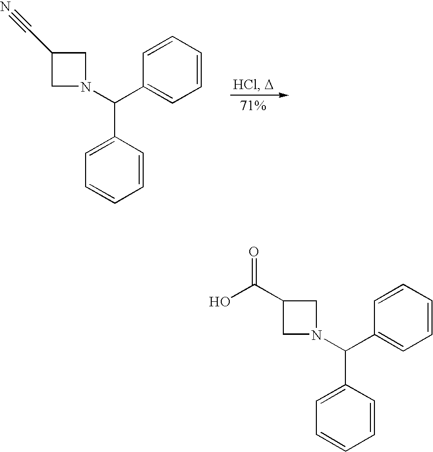

A suspension of 1-benzhydryl-azetidine-3-carbonitrile (2.09 g, 8.42 mmol) in concentrated hydrochloric acid (12 M, 15 mL) was heated at reflux for 30 minutes. The resulting solution was cooled to 0° C., and 6 M sodium hydroxide was added until the mixture reached a pH of about 7. The aqueous mixture was then extracted with dichloromethane (3×150 mL) and dichloromethane:methanol (10:1, 3×150 mL). The combined organic layers were dried, filtered, and concentrated under reduced pressure to give the title compound (1.60 g, 71% yield). MS (APCI): m/z 268 (M+H)⁺.

### B. 1-Benzhydryl-azetidine-3-carboxylic acid methoxymethylamide

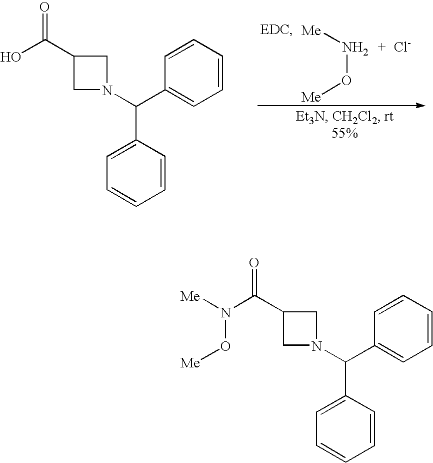

1-[3-(Dimethylamino)propyl]-3-ethylcarbodiimide hydrochloride (8.0 g, 42 mmol) was added to a suspension of 1-benzhydryl-azetidine-3-carboxylic acid (7.42 g, 27.8 mmol), N,O-dimethyl-hydroxyl-amine hydrochloride (4.24 g, 43.5 mmol), and triethylamine (11.6 mL, 83.3 mmol) in dichloromethane (150 mL). The suspension was then stirred at room temperature for 60 minutes. The suspension was diluted with dichloromethane (300 mL), and the resulting solution washed with water (3×100 mL). The organic layer was then dried (magnesium sulfate), filtered, and concentrated under reduced pressure. The resulting solid was purified via medium pressure liquid chromatography eluting with dichloromethane:methanol (40:1) to deliver 4.76 g (55% yield) of the title compound as a white solid (mp 103-106° C.); MS (APCI+): m/z 311 (M+H)⁺.

### C. 1-(1-Benzhydryl-azetidin-3-yl)-propan-1-one

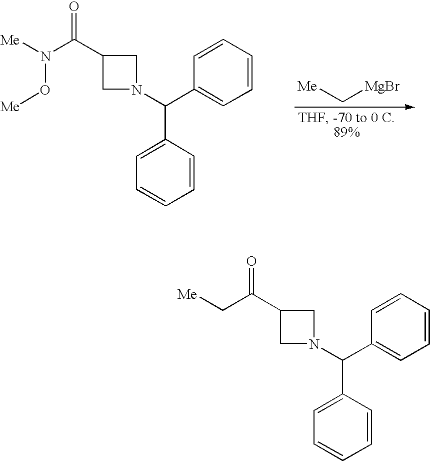

A solution of ethylmagnesium bromide in tetrahydrofuran (1.0 M, 32.5 mL) was add to a solution of 1-benzhydryl-azetidine-3-carboxylic acid methoxymethylamide (3.36 g, 10.8 mmol) at −70° C. in tetrahydrofuran (60 mL). The resulting reaction mixture was then stirred at 0° C. for 1 hour. The reaction was then poured into a saturated aqueous solution of ammonium chloride (75 mL) at 0° C. The mixture was then extracted with diethyl ether (300 mL); the aqueous layer was then extracted again with diethyl ether (2×100 mL). The combined organic layers were dried over magnesium sulfate, filtered, and concentrated under reduced pressure. The crude product was purified via medium pressure chromatography eluting with dichloromethane:methanol (40:1) to afford 2.69 g (89% yield) of the title compound as a waxy yellow solid (mp 73-75° C.); MS (APCI+): m/z 280 (M+H)⁺.

### D. N-[1-(1-Benzhydryl-azetidin-3-yl)propyl]-2,2,2-trifluoroacetamide

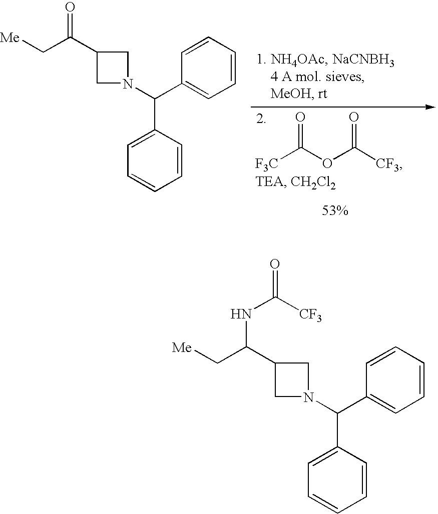

Ammonium acetate (6.00 g, 77.8 mmol) was added to a mixture of 1-(1-benzhydryl-azetidin-3-yl)-propan-1-one (2.59 g, 9.27 mmol) and 4 angstrom molecular sieves (2.60 g) in methanol (80 mL). The mixture was cooled to 0° C., and sodium cyanoborohydride (1.17 g, 18.5 mmol) was added in several portions. The mixture was then stirred at room temperature for 22 hours. The suspension was filtered, and filtrate was concentrated under reduced pressure. The resulting residue was partially dissolved in dichloromethane (500 mL). The mixture was then washed with saturated aqueous sodium carbonate (100 mL). The layers were separated, and the aqueous layer was extracted with dichloromethane (100 mL). The combined organic layers were dried over magnesium sulfate, filtered, and concentrated under reduced pressure to give 2.59 g of a clear oil which was used without further purification.

A solution of the crude diamine (2.59 g) and triethylamine (3.86 mL, 27.7 mmol) in dichloromethane (60 mL) at 0° C. was treated with trifluoroacetic anhydride (1.06 mL, 13.9 mmol). The resulting solution was then stirred at room temperature for 45 minutes. After 45 minutes, an additional amount of trifluoroacetic anhydride (350 μL) was added, and stirring continued for 15 minutes at room temperature. The solution was cooled to 0° C., and saturated aqueous sodium bicarbonate (10 mL) was added. The mixture was then partitioned between dichloro-methane (300 mL) and saturated aqueous bicarbonate (40 mL). The layers were separated; the organic layer washed with water (50 mL), dried over magnesium sulfate, filtered, and concentrated under reduced pressure. The resulting oil was purified via medium pressure liquid chromatography eluting with a gradient of hexanes:ethyl acetate (80:20 to 60:40) to give 1.84 g (53% yield) of the title compound. MS (APCI+) m/z 377 (M+H)⁺.

### E. N-(1-Azetidin-3-yl-propyl)-2,2,2-trifluoroacetamide hydrochloride

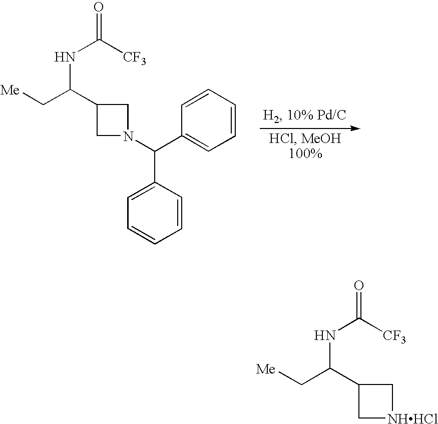

A mixture of N-[1-(1-benzhydryl-azetidin-3-yl)propyl]-2,2,2-trifluoroacetamide (1.72 g, 4.57 mmol), 10% Pd/C (2.02 g), concentrated hydrochloric acid (12.0 M, 0.380 mL) in methanol (60 mL) was hydrogenated at 50 psi for 6 hours. An additional amount of 10% Pd/C (1.5 g) was added, and the hydrogenation continued for 22 hours. The solvent was removed under reduced pressure to deliver a yellow residue. The residue was then concentrated several times with toluene and then dried in vacuo at 50° C. for several hours to deliver the title compound contaminated with diphenylmethane. The crude material was triturated with hexanes to provide 1.13 g, (100% yield) of the title compound. MS (APCI+): m/z 211 (M+H)⁺.

### EXAMPLE 5

### Preparation of 2,2,2-Trifluoro-N-(3-methyl-azetidin-3-ylmethyl)-acetamide hydrochloride

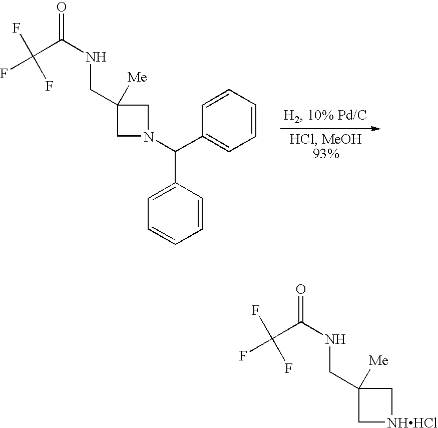

A mixture of N-(1-Benzhydryl-3-methyl-azetidin-3-ylmethyl)-2,2,2-trifluoro-acetamide (3.19 g, 8.80 mmol), 10% Pd/C (2.5 g), concentrated hydrochloric acid (12 M, 0.732 mL) in methanol (50 mL) was hydrogenated at 50 psi for 8 hours. The solvent was removed under reduced pressure to deliver a yellow residue. The residue was then concentrated several times with toluene. The resulting solid was then triturated with hexanes, and the supernatant was discarded. The resulting white solid was dried in vacuo to provide 1.91 g (93% yield) of the title compound. MS (APCI+): m/z 197 (M+H)⁺.

### EXAMPLE 6

### Preparation of (R) and (S)-1-Pyrrolidin-3-yl-cyclopropylamine

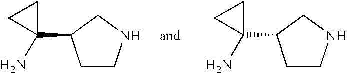

### A. N,N-Dibenzylacrylamide

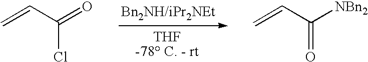

A round bottom flask was charged with tetrahydrofuran (3750 mL) and cooled to −78° C. under nitrogen. Acryloyl chloride (55.7 g, 48.9 mL, 0.615 mol) and diisopropylethylamine (87.3 g, 118 mL, 0.676 mol) were added followed by the slow addition (over a period of 20 minutes) of dibenzylamine (109.6 g, 106 mL, 0.555 mol). The reaction mixture was allowed to warm up to room temperature and stirred at room temperature for 1.0 hour. A large quantity of white precipitate was observed and thin layer chromatography indicated the reaction was complete. The solids were removed by filtration and the filtrate was concentrated under vacuum to afford a quantitative yield of the title compound.

### B. N-(Trimethylsilylmethyl)-α-methylbenzylamine

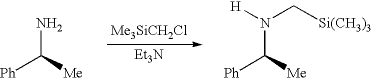

A mixture of (S)-(−)-α-methylbenzylamine (100 g, 106.4 mL, 0.82 mol), chloromethyltrimethylsilane (115.1 mL, 101.2 g, 0.82 mol), and triethylamine (126.5 mL, 96.2 g, 0.95 mol) was heated at reflux for 24 hours until LCMS indicated the reaction was complete. The reaction mixture was triturated with heptane and the HCl salt was filtered off. The heptane filtrate was concentrated to an oily residue which was distilled under vacuum (42-50° C./0.4-0.7 mmHg) to furnish 67.8 g (40% yield) of the title compound.

### C. N-(Methoxymethyl)-N-(trimethylsilylmethyl)-α-methylbenzylamine

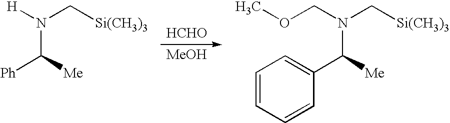

To a stirred solution of aqueous formaldehyde (37%, 152.1 g, 1.9 mol) at 0° C. was added the N-(trimethylsilylmethyl)-α-methylbenzylamine from the previous step (310 g, 1.5 mol) over a period of 0.5 hour followed by the addition of methanol (100 mL) and potassium carbonate (200 g). The reaction mixture was stirred at 0-10° C. for 1-2 hours. The mixture was filtered and the filtrate was extracted with diethyl ether (1 time). The ether layer was dried with sodium sulfate and concentrated to an oil which was distilled using a Kugelrohl apparatus to furnish 210 g (56%) of the title compound.

### D. 1-(1-Phenyl ethyl)-pyrrolidine-3-carboxylic acid dibenzylamide

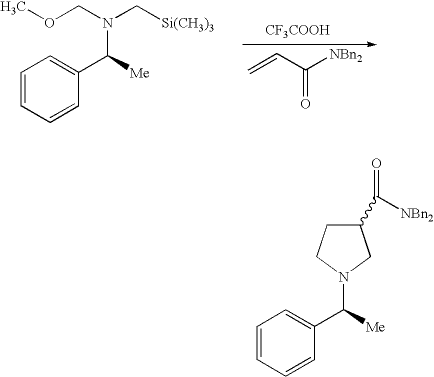

N,N-Dibenzylacrylamide (79.5 g, 0.317 mol) and N-(methoxymethyl)-N-(trimethylsilylmethyl)-α-methylbenzylmine (103 g, 0.412 mol) were dissolved in CH₂Cl₂ (1500 mL) and cooled to 0° C. Trifluoroacetic acid solution (1.0 M in CH₂Cl₂, 27 mL) was added over a period of 20 minutes and the resulting reaction mixture was stirred at room temperature overnight. The mixture washed with aqueous NaHCO₃, brine, dried over Na₂SO₄ and concentrated. The residue was purified by flash chromatography (heptane-EtOAc-Et₃N/10:2:0.1) to furnish 97.7 g of the title compound (77% yield) as a mixture of two diasteromers.

### E. Dibenzyl-{1-[1-(1-phenyl-ethyl)-pyrrolidin-3-yl]-cyclopropyl}-amine

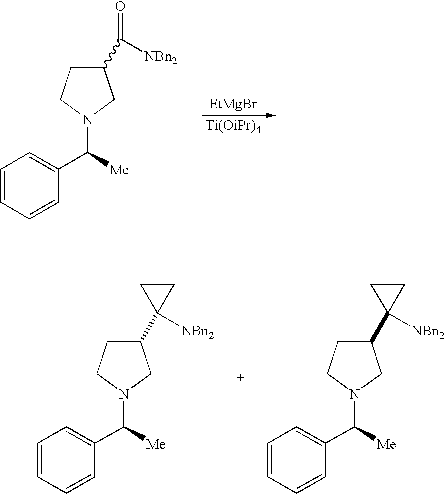

To a round bottom flask charged with tetrahydrofuran (1400 mL) was added ethylmagnesium bromide (EtMgBr) (3.0 M in Et₂O, 178 mL, 0.534 mol) at −78° C. A solution of Ti(OiPr)₄ (64.8 g, 66.0 mL, 0.228 mol) in THF (150 mL) was added then added at a rate to maintain the temperature below −68° C. The dark solution was allowed to stir at −68° C. for 3 minutes before a solution of 1-(1-phenyl-ethyl)-pyrrolidine-3-carboxylic acid dibenzylamide (86.6 g, 0.218 mol) in THP (150 mL) was added below −68° C. The reaction mixture was allowed to warm to room temperature, and then was stirred at room temperature for 1.0 hour and then was heated at reflux for 1.0 hour. The reaction mixture was then cooled to 8° C. EtMgBr (3.0 M in ether, 150 mL, 0.450 mol) was added followed by the rapid addition of Ti(OiPr)₄ (54.6 g, 55.6 mL, 0.192 mol) in THF (150 mL). The resulting mixture was stirred at room temperature for 1.0 hour before it was quenched with aqueous ammonium chloride (3000 mL) and water (800 mL). The mixture was filtered through Celite, rinsed with ether. The organic layer was separated. The aqueous layer was made basic (pH˜8.5) with aqueous NaOH and extracted with ether. The combined organic layers were dried over Na₂SO₄, concentrated and purified by flash chromatograph (heptane-EtOa\Ac-Et₃N/10:1:0.1) to provide the title compound as a mixture of stereoisomers, which were separated prior to subsequent transformations. Isomer 1 (31.3 g, 35%) as colorless crystals (mp 76-76.5° C.). The stereochemical structure of isomer 1 was confirmed by a single crystal X-ray diffraction experiment.

Isomer 2: The impure oil (18 g) from the above purification was further chromatographed with heptane/methylbutyl ether (MTBE)/Et₃N (100:0.5:0.5) to furnish 11 g of isomer 2 which was about 90% pure as a colorless oil. This oil was dissolved in Et₂O (350 mL) and titrated with 2.0 M Et₂O—HCl (12.8 mL). The resulting white solid was collected by filtration, rinsed with ether, dissolved in MeOH, neutralized with 15% NaOH, extracted with ether (2 times), washed with brine, dried over Na₂SO₄, concentrated under vacuum to afford a thick oil which was recrystallized in EtOH at −30° C. to furnish 10.1 g of the title compound (22% yield) as colorless crystals (mp 61-61.3° C.).

### F. S-1-Pyrrolidin-3-yl-cyclopropylamine

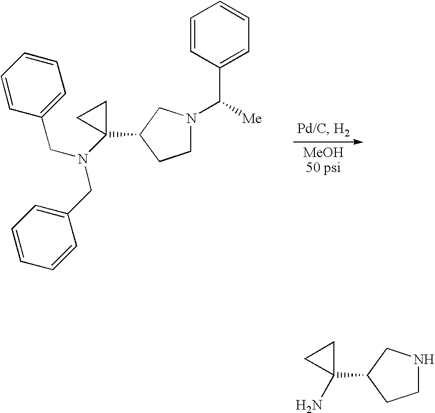

Dibenzyl-{1-[1-(1-phenyl-ethyl)-pyrrolidin-3-yl]-cyclopropyl}-amine (3.00 g, 7.32 mmol) was charged with 20% Pd/C and subjected to 50 psi hydrogenation conditions. After 48 hours the reaction was filtered and concentrated to give 764 mg of the title compound. (yield: 83%). MS (APCI+): m/z 127 (M+H)⁺.

### G. (R)-1-Pyrrolidin-3-yl-cyclopropylamine

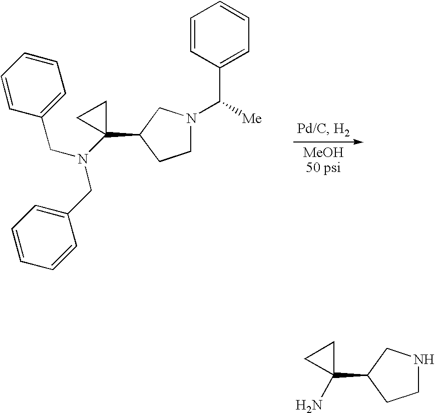

Dibenzyl-{1-[1-(1-phenyl-ethyl)-pyrrolidin-3-yl]-cyclopropyl}-amine (3.01 g, 7.32 mmol) was charged with 20% Pd/C and subjected to 50 psi hydrogenation conditions. After 48 hours the reaction was filtered and concentrated to give 844 mg of the title compound. (yield: 91%). MS (APCI+): m/z 127 (M+H)⁺.

### EXAMPLE 7

### Preparation of Pyrrolidin-3-ylmethyl-carbamic acid tert-butyl ester

### A. (S)-Methanesulfonic acid 1-benzyl-pyrrolidin-3-yl ester

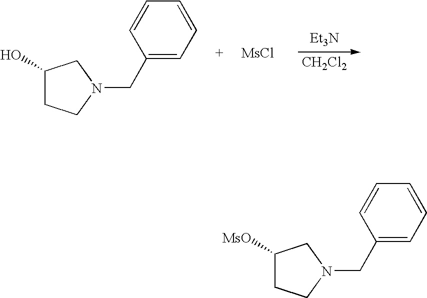

1-Benzyl-pyrrolidin-3-ol (Synthetic Communications, 1985) (25.01 g, 141 mmol) was taken up in dichloromethane and charged with triethylamine (29 mL). The resulting solution was cooled to 0° C. and charged with mesyl chloride (13.1 mL). After 14 hours the reaction washed with saturated sodium bicarbonate followed with water and brine. The organic layer was dried and concentrated to give the title compound (30.2 g, 84% yield). MS (APCI+): m/z 256 (M+H)⁺.

### B. (R)-1-Benzyl-pyrrolidine-3-carbonitrile

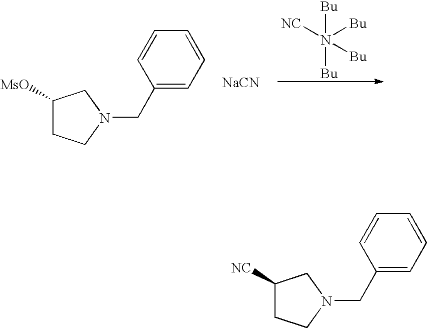

(R)-1-Benzyl-pyrrolidine-3-carbonitrile (29.8 g, 117 mmol) was taken up in acetonitrile and charged with sodium cyanide (20.2 g, 412 mmol) and tetra butyl ammonium cyanide (3.11 g, 11.6 mmol) then heated to reflux. After 48 hours the reaction was diluted with ethyl acetate and washed with saturated sodium bicarbonate, water and brine. The organic layer was dried, concentrated and purified via column chromatography (3:1 hexanes/ethyl acetate) leaving 15.4 g of the title compound (71% yield). MS (APCI+): m/z 187 (M+H)⁺.

### C. (R) —C-(1-Benzyl-pyrrolidin-3-yl)-methylamine

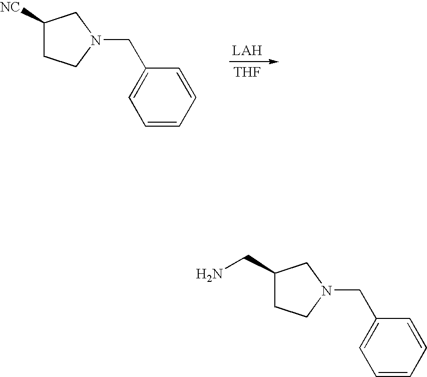

1-Benzyl-pyrrolidine-3-carbonitrile (5.08 g, 27.3 mmol) was taken up in THF and cooled to 0° C. After 10 minutes LAH (2.09 g, 55.1 mmol) in THF at 0° C. was slowly added to the pyrrolidine solution. Gas evolution was observed, and the reaction was allowed to continue at 0° C. for 30 minutes. The reaction was allowed to warm to room temperature and stirred for 2 additional hours. The reaction was quenched with water (2 mL), 1N NaOH (2 mL), and again water (6 mL). The resulting slurry was filtered over a pad of celite which washed with dichloromethane and the combined filtrates were concentrated to give 4.2 g of the title compound (yield: 82%). MS (APCI+): m/z 191 (M+H)⁺.

### D. (R)-(1-Benzyl-pyrrolidin-3-ylmethyl)-carbamic acid tert-butyl ester

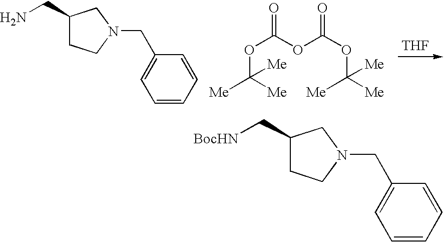

R-C-(1-Benzyl-pyrrolidin-3-yl)-methylamine (2.033 g, 10.7 mmol) was taken up in THF and charged with boc anhydride (6.787 g, 31 mmol). The resulting solution was heated gently to 46° C. After 6 hours the resulting solution was cooled to room temperature and concentrated. The crude residue was taken up in dichloromethane and washed with 1.0 N HCl. The organic washes were purified via chromatography (0-10% MeOH/CH₂Cl₂) to provide 2.4 g of the title compound (yield: 76%). MS (APCI+): m/z 291 (M+H)⁺.

### E. Pyrrolidin-3-ylmethyl-carbamic acid tert-butyl ester

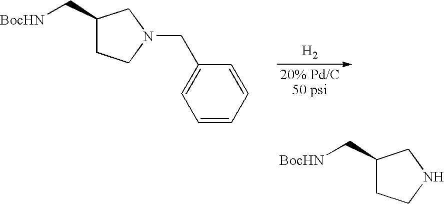

(R)-(1-Benzyl-pyrrolidin-3-ylmethyl)-carbamic acid tert-butyl ester (1.00 g, 3.44 mmol) was charged with 20% Pd/C and hydrogenated at 50 psi. After 48 hours, the reaction was filtered and concentrated to give 511 mg of the title compound (yield: 74%). MS (APCI+): m/z 201 (M+H)⁺.

### EXAMPLE 8

### Preparation of Pyrrolidin-3-ylmethyl-carbamic acid tert-butyl ester

### A. [1-(1-Phenyl-ethyl)-pyrrolidin-3-yl]-methanol

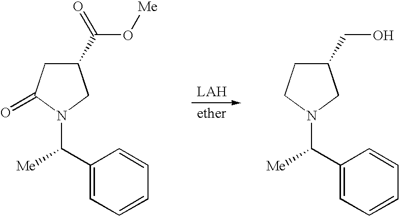

5-Oxo-1-(1-phenyl-ethyl)-pyrrolidine-3-carboxylic acid methyl ester (Journal of Heterocyclic Chemistry, 1992) (10.0 g, 40.5 mmol) was taken up in diethyl ether and added slowly to a slurry of LAH (2.31 g, 60.86 mmol) in diethyl ether. The resulting solution was heated at reflux for 4 hours. The reaction was cooled to room temperature and quenched with a water/ether mixture. The resulting solution was allowed to stir for 1 additional hour at room temperature. The slurry was filtered and washed with dichloromethane. The filtrates were concentrated at reduced pressure to give 7.76 g of the title compound (yield: 94%). MS (APCI+): m/z 206 (M+H)⁺.

### B. 2-[1-(1-Phenyl-ethyl)-pyrrolidin-3-ylmethyl]-isoindole-1,3-dione

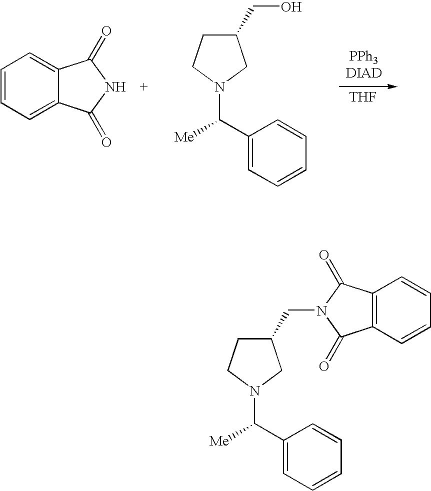

[1-(1-Phenyl-ethyl)-pyrrolidin-3-yl]-methanol (4.4 g, 21.5 mmol) was taken up in THF and charged with triphenyl phosphine (6.27 g, 23.9 mmol), and phthalimide (3.61 g, 24.6 mmol) followed by diisopropylazodicarboxylate (DIAD) (5.08 g, 25.1 mmol) dropwise. After 4 hours the reaction was concentrated and the resulting oil was chromatographed (1-10% isopropyl alcohol/dichloromethane) to provide 5.6 g of the title compound (yield: 77%). MS (APCI+): m/z 335 (M+H)⁺.

### C. C-[1-(1-Phenyl-ethyl)-pyrrolidin-3-yl]-methylamine

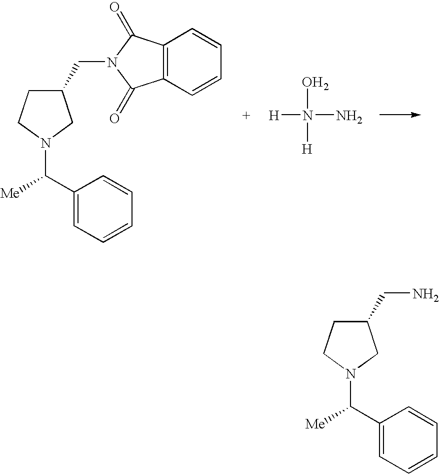

The phthalimide (5.00 g, 14.9 mmol) was taken up in isopropyl alcohol and charged with hydrazine hydrate (7.04 g, 149 mmol). The resulting solution was heated to 60° C. After 1 hour a colorless precipitate had formed. The reaction was diluted with isopropyl alcohol and filtered. The filter cake washed with isopropyl alcohol and the combined filtrates were concentrated to give an off white oily solid. This residue was partitioned between water and 1:3 dichloromethane:ether and the organic layer washed with water then dried over sodium sulfate to give 1.68 g of the title compound (yield: 55%): MS (APCI+): m/z 205 (M+H)⁺.

### D. [1-(1-Phenyl-ethyl)-pyrrolidin-3-ylmethyl]-carbamic acid tert-butyl ester

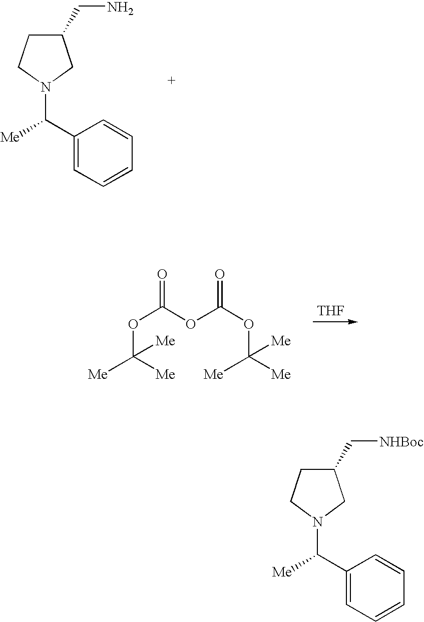

C-[1-(1-Phenyl-ethyl)-pyrrolidin-3-yl]-methylamine (4.01 g, 19.6 mmol) was taken up in THF and charged with boc anhydride (15.3 g, 70.1 mmol). The resulting solution was heated gently to 50° C. After 6 hours the resulting solution was cooled to room temperature and concentrated. The crude was taken up in dichloromethane and washed with 1N HCl. The organics were concentrated to provide the title compound 3.4 g (yield: 58%). MS (APCI+): m/z 305 (M+H)⁺.

### E. Pyrrolidin-3-ylmethyl-carbamic acid tert-butyl ester

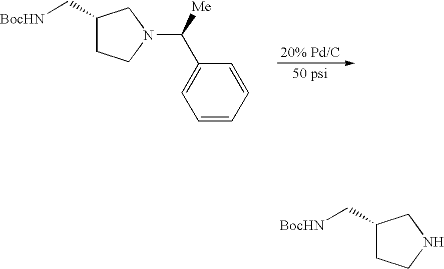

[1-(1-Phenyl-ethyl)-pyrrolidin-3-ylmethyl]-carbamic acid tert-butyl ester (3.50 g, 11.5 mmol) was taken up in methanol and charged with 20% Pd/C then subjected to 50 psi hydrogenation conditions. After 24 hours the reaction was filtered and concentrated leaving 1.75 g of title compound (yield: 76%). MS (APCI+): m/z 201 (M+H)⁺.

### EXAMPLE 9

### Preparation of C-Oxazol-2-yl-C-pyrrolidin-3-yl-methylamine

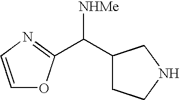

### A. 3-(Hydroxy-oxazol-2-yl-methyl)-pyrrolidine-1-carboxylic acid benzyl ester

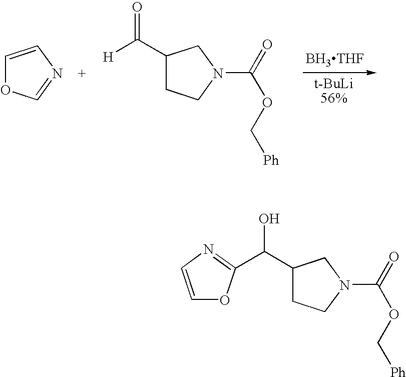

To a solution of oxazole (2.0 g, 29 mmol) in tetrahydrofuran (30 mL) was added borane-tetrahydrofuran complex (32 mL, 1M in THF) dropwise at room temperature. The reaction mixture was cooled to −78° C. and tert-butyllithium (19 mL, 1.7 M in hexanes) was added dropwise. After stirring for 30 minutes, a solution of 3-formyl-pyrrolidine-1-carboxylic acid benzyl ester (2.0 g, 29 mmol) in tetrahydrofuran (5 mL) was added. The reaction mixture was stirred at −78° C. for 5 h, then 5% acetic acid in ethanol (180 mL) was added. The mixture was warmed to room temperature, poured into brine and extracted three times with ethyl acetate. The combined organic layers were dried over magnesium sulfate, filtered and concentrated in vacuo. The crude residue was purified by flash column chromatography (40% to 100% ethyl acetate in hexanes) to afford the title compound (4.9 g, 56%). MS (APCI+): m/z 303 (M+H)⁺.

### B. 3-(Azido-oxazol-2-yl-methyl)-pyrrolidine-1-carboxylic acid benzyl ester

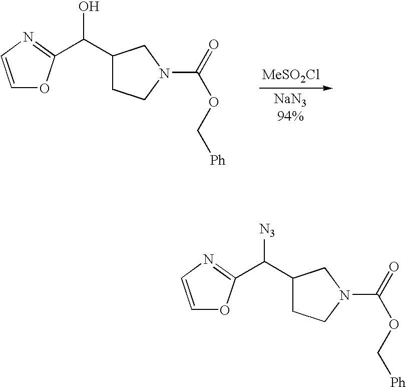

To a cooled (0° C.) solution of 3-(hydroxy-oxazol-2-yl-methyl)-pyrrolidine-1-carboxylic acid benzyl ester (4.9 g, 16 mmol) in dichloromethane (80 mL) was added triethylamine (2.9 mL, 21 mmol), followed by methanesulfonyl chloride (1.51 mL, 19.4 mmol). The solution was warmed to room temperature and stirred overnight. Dichloromethane was added, and the solution washed with saturated aqueous sodium chloride. The organic layer was dried over magnesium sulfate, filtered, and concentrated. The resulting mesylate was used in the next step without further purification.

To a solution of the crude mesylate in N,N-dimethylformamide (80 mL) was added sodium azide (10 g, 160 mmol): The resulting mixture was heated at 80° C. overnight. The reaction mixture was cooled to room temperature, poured into water, and extracted with ethyl acetate. The organic layer was dried over magnesium sulfate, filtered, and concentrated in vacuo. The crude residue was purified by flash column chromatography (0 to 40% ethyl acetate in hexanes) to afford the title compound (4.9 g, 94%) as a colorless oil. MS (APCI+): m/z 328 (M+H)⁺.

### C. 3-(Amino-oxazol-2-yl-methyl)-pyrrolidine-1-carboxylic acid benzyl ester

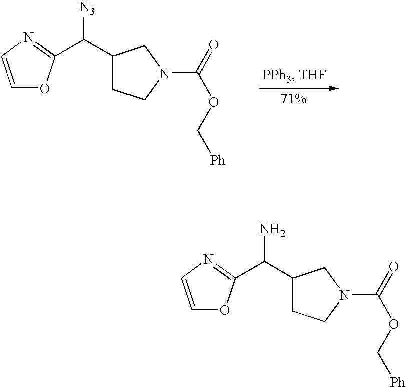

To a solution of 3-(azido-oxazol-2-yl-methyl)-pyrrolidine-1-carboxylic acid benzyl ester (1.0 g, 3.1 mmol) in tetrahydrofuran (20 mL) was added triphenylphosphine (1.85 g, 7.03 mmol) and water (0.60 mL, 31 mmol), and the mixture was allowed to stir at 50° C. for 18 hours. The reaction mixture was cooled to room temperature and concentrated in vacuo. The resulting residue was purified by flash chromatography (1:9 methanol/dichloromethane) to afford the title compound (0.66 g, 71%). ¹H NMR (400 M, CDCl₃) δ 7.64-7.01 (m, 7H), 5.19-5.08 (m, 2H), 4.01-3.12 (m, 5H), 2.74-2.53 (m, 1H), 2.21-1.55 (m, 4H).

### D. C-Oxazol-2-yl-C-pyrrolidin-3-yl-methylamine

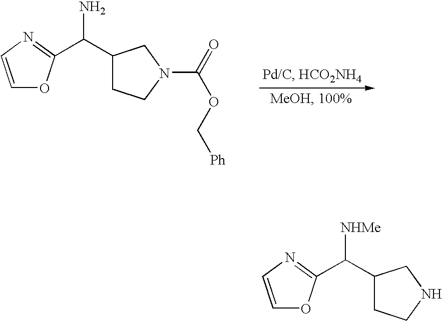

To a solution of 3-(Amino-oxazol-2-yl-methyl)-pyrrolidine-1-carboxylic acid benzyl ester (0.65 g, 2.2 mmol) in methanol (10 mL) was added ammonium formate (0.68 g, 11 mmol) and 10% palladium on carbon (0.70, 0.65 mmol). The reaction mixture was heated at 65° C. for 2.5 hours, cooled to room temperature, and filtered. The filtrate was concentrated in vacuo to afford the title compound (0.36 g, 100%). ¹H NMR (400 M}, CDCl₃) δ 7.95 (s, 1H), 7.14 (s, 1H), 4.04-3.92 (m, 1H), 3.39-2.58 (m, 7H), 2.18-1.51 (m, 3H).

### EXAMPLE 10

### Preparation of (2-Cyano-1-pyrrolidin-3-yl-ethyl)-carbamic acid tert-butyl ester

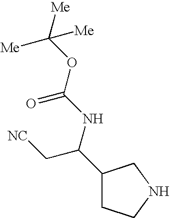

### A. 3-(1-tert-Butoxycarbonylamino-2-cyano-ethyl)-pyrrolidine-1-carboxylic acid benzyl ester

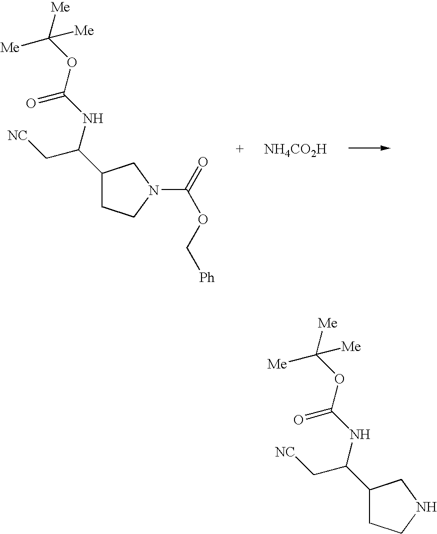

The 3-(1-tert-butoxycarbonylamino-2-cyano-ethyl)-pyrrolidine-1-carboxylic acid benzyl ester isomeric mixture was first purified over silica gel column with 25 to 75% ethyl acetate in hexanes over 50 minutes to give diastereomers A and B. Diastereomer A was subjected to chiral HPLC (Chiralpak AD, 10% ethanol in methanol) to give enantiomers A1 (8.4 minutes) and A2 (12.2 minutes).

To a solution of 3-(1-tert-butoxycarbonylamino-2-cyano-ethyl)-pyrrolidine-1-carboxylic acid benzyl ester (0.53 g, 1.41 mmol) in methanol (25 mL) under nitrogen atmosphere were added ammonium formate (0.27 g, 4.23 mmol) and 10% Pd/C (0.25 g). The nitrogen source was removed and the reaction flask was capped. After 2 days, the reaction mixture was filtered through celite and the filtrate was concentrated in vacuo to give 0.34 g of title compound as a mixture of isomers (100%). MS (APCI+): m/z 240 (M+H)⁺.

### EXAMPLE 11

### Preparation of Pyrrolidin-3-yl-acetonitrile

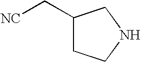

### A. 3-(Toluene-4-sulfonyloxymethyl)-pyrrolidine-1-carboxylic acid benzyl ester

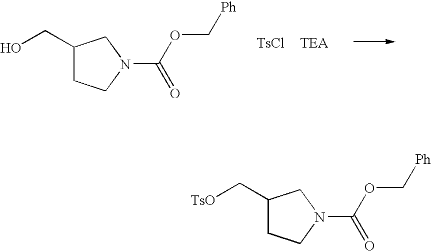

To a solution of 3-hydroxymethyl-pyrrolidine-1-carboxylic acid benzyl ester (1.80 g, 7.65 mmol) in dichloromethane (10 mL) were added triethylamine (1.60 mL, 11.48 mmol) and p-toluenesulfonyl chloride (1.75 g, 9.18 mmol). After 3 hours, the reaction mixture washed with saturated sodium bicarbonate, water and brine. The organic layer was dried over MgSO₄, filtered and filtrate concentrated. Purification via flash column chromatography (ethyl acetate/hexanes gradient) afforded 2.63 g of the title compound (88% yield). MS (APCI+): m/z 390 (M+H)⁺.

### B. 3-Cyanomethyl-pyrrolidine-1-carboxylic acid benzyl ester

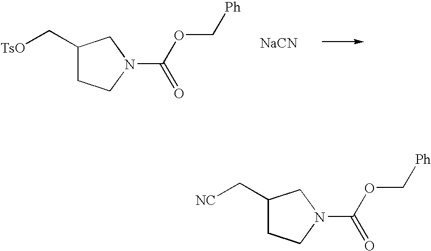

To a solution of 3-(toluene-4-sulfonyloxymethyl)-pyrrolidine-1-carboxylic acid benzyl ester (1.52 g, 3.90 mmol) in DMSO (3 mL) was added sodium cyanide (0.25 g, 5.07 mmol). The reaction mixture was heated to 70° C. After 4 hours, the reaction mixture was poured into saturated sodium bicarbonate and extracted with ethyl acetate. The organic layer was dried over MgSO₄, filtered and the filtrate was concentrated at reduced pressure. Purification via flash column chromatography (ethyl acetate/hexanes gradient) afforded 0.81 g of the title compound (85%). MS (APCI+): m/z 245 (M+H)⁺.

### C. Pyrrolidin-3-yl-acetonitrile

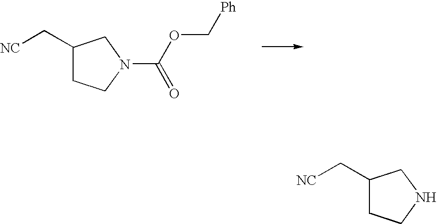

To a solution of 3-cyanomethyl-pyrrolidine-1-carboxylic acid benzyl ester (0.80 g, 3.27 mmol) in methanol (50 mL) were added triethylamine (0.5 mL) and 10% Pd/C (0.2 g). The reaction vessel was pressurized to 50 psi for 24 hours, filtered through celite, and the filtrate was concentrated at reduced pressure to give 0.36 g of the title compound (100% yield). MS (APCI+): m/z 111 (M+H)⁺.

### EXAMPLE 12

### Preparation of 3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-2,2-dimethyl-propionitrile Hydrochloride salt

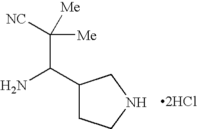

### A. Pyrrolidine-3-carboxylic acid ethyl ester

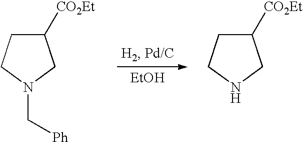

A solution of 1-benzyl-pyrrolidine-3-carboxylic acid ethyl ester (10.00 g, 42.9 mmol) in ethanol (200 mL) was hydrogenated in the presence of 10% Pd/C (2.0 g) at 60 psi for 6 hours. The resulting suspension was filtered through celite, washed with CH₂Cl₂, and concentrated under reduced pressure to leave the crude title compound (7.12 g, 100% yield). ¹H NMR □(CDCl₃) 4.16 (q, 2H), 3.02-3.17 (m, 3H), 2.82-2.94 (m, 2H), 1.91-2.07 (m, 2H), 1.26 (t, 3H). LCMS (APCI⁺) 144 (100%, MH⁺).

### B. Pyrrolidine-1,3-dicarboxylic acid 1-tert-butyl ester 3-ethyl ester

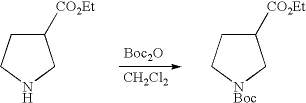

To a solution of crude pyrrolidine-3-carboxylic acid ethyl ester (7.12 g) in CH₂Cl₂ (50 mL) at 0° C. was added a solution of di-tert-butyl dicarbonate (10.30 g, 47.2 mmol) in CH₂Cl₂ (50 mL) over 10 minutes. After warming to room temperature over 18 hours, the reaction mixture washed with water, then brine, dried (Na₂SO₄) and concentrated under reduced pressure to leave the title compound which was used without further purification (10.4 g, 100% yield). ¹H NMR □(CDCl₃) 4.14 (q, 2H), 3.27-3.69 (m, 4H), 3.02 (m, 1H), 2.07-2.16 (m, 2H), 1.46 (s, 9H), 1.27 (t, 3H).

### C. 3-Hydroxymethyl-pyrrolidine-1-carboxylic acid tert-butyl ester

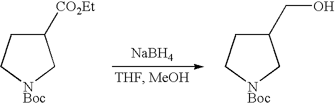

To a solution of pyrrolidine-1,3-dicarboxylic acid 1-tert-butyl ester 3-ethyl ester (10.4 g, 42.9 mmol) in tetrahydrofuran (50 mL) and methanol (50 mL) at 0° C. was added sodium borohydride (NaBH) (3.25 g, 86 mmol) in portions over 30 minutes. After 18 hours, more NaBH (3.25 g, 86 mmol) was added. After a further 24 hours, the reaction mixture was diluted with ethyl acetate, quenched with saturated aqueous Na₂CO₃ and stirred for 15 minutes. The layers were separated, the aqueous layer extracted with ethyl acetate, and then the combined organic layers washed twice with water, once with brine, dried (Na₂SO₄) and concentrated under reduced pressure. The crude product was purified by column chromatography (CH₂Cl₂ to CH₂Cl₂:MeOH 95:5 to 9:1) to give the title compound (8.09 g, 94% yield). ¹H NMR □(CDCl₃) 3.25-3.69 (m, 5H), 3.11 (m, 1H), 2.40 (m, 1H), 1.97 (m, 1H), 1.67 (m, 1H), 1.46 (s, 9H).

### D. 3-Formyl-pyrrolidine-1-carboxylic acid tert-butyl ester

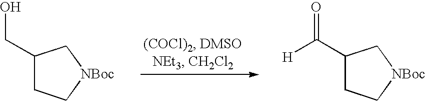

To a solution of oxalyl chloride (3.86 mL, 44.2 mmol) in CH₂Cl₂ (80 mL) at −78° C. under N₂ was added a solution of dimethyl sulfoxide (6.28 mL, 88.5 mmol) in CH₂Cl₂ (20 mL). After 10 minutes, a solution of 3-hydroxymethyl-pyrrolidine-1-carboxylic acid tert-butyl ester (8.09 g, 40.2 mmol) in CH₂Cl₂ (30 mL) was added over 15 minutes. After a further 30 minutes, triethylamine (28.0 mL, 201 mmol) was added, and the reaction mixture was stirred for 1 hour at −78° C. then 1 hour at room temperature. The reaction mixture washed twice with water then with brine, dried (Na₂SO₄) and concentrated under reduced pressure. The crude product was purified by column chromatography (hexanes:ethyl acetate 9:1 to 1:1) to give the title compound (6.98 g, 87%). ¹H □(CDCl₃) 9.69 (d, J=1.7 Hz, 1H), 3.26-3.80 (m, 4H), 3.03 (m, 1H), 2.02-2.29 (m, 2H), 1.46 (s, 9H).

### E. 3-(Benzenesulfonyl-tert-butoxycarbonylamino-methyl)-pyrrolidine-1-carboxylic acid tert-butyl ester

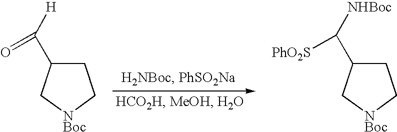

To a suspension of tert-butyl carbamate (589 mg, 5.03 mmol) and sodium benzenesulfinate (1.24 g, 7.55 mmol) in water (50 mL) was added a solution of 3-formyl-pyrrolidine-1-carboxylic acid tert-butyl ester (1.00 g, 5.03 mmol) in methanol (5 mL), followed by formic acid (0.19 mL, 5.03 mmol). The reaction mixture was heated to 60° C. for 2 hours, then stood at room temperature for 7 days. The resulting white solid was filtered off, washed with water and dried thoroughly under reduced pressure to give the title compound (868 mg, 39% yield). ¹H NMR □(CDCl₃) 7.91 (d, 2H), 7.50-7.68 (m, 3H), 4.82-5.18 (m, 2H), 3.71 (m, 1H), 3.54 (m, 1H), 3.31 (m, 1H), 2.90-3.19 (m, 2H), 2.35 (m, 0.5H), 2.18 (m, 0.5H), 1.76-1.99 (m, 1H), 1.47 (s, 9H), 1.21 (s, 4.5H), 1.18 (s, 4.5H.

### F. 3-(1-tert-Butoxycarbonylamino-2-cyano-2,2-dimethyl-ethyl)-pyrrolidine-1-carboxylic acid tert-butyl ester

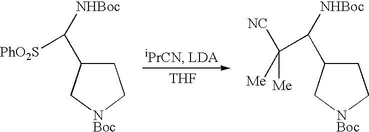

To solution isobutyronitrile (4.07 mL, 45 mmol) in dry THF (100 mL) at −78° C. under an atmosphere of nitrogen was added lithium diisopropylamide (30.3 mL of a 1.5 M solution in cyclohexane, 45 mmol). After 1 hour, this solution was transferred by cannula to a stirred suspension of 3-(benzenesulfonyl-tert-butoxycarbonylamino-methyl)-pyrrolidine-1-carboxylic acid tert-butyl ester (2.00 g, 4.55 mmol) in dry THF (100 mL) at −78° C. After 7 hours, the reaction was slowly warmed to room temperature overnight. The reaction was then quenched with saturated aqueous ammonium chloride (NH₄Cl) and extracted twice with CH₂Cl₂. The organic phase washed with saturated aqueous NaHCO₃, then dried (Na₂SO₄) and concentrated under reduced pressure. The crude product was purified by column chromatography, firstly with hexanes: EtOAc 3:1 to 2:1, and then with CH₂Cl₂: MeOH 99.5:0.5 to 99:1) to give the title compound (1.52 g, 91% yield). ¹H NMR □(CDCl₃) 4.64-4.79 (m, 1H), 3.42-3.85 (m, 3H), 2.93-3.29 (m, 2H), 2.54 (m, 1H), 1.96-2.14 (m, 1H), 1.74-1.80 (m, 1H), 1.35-1.47 (m, 24H). LCMS (APCI⁻) 366 (100%, (M−H)⁻).

### G. 3-Amino-2,2-dimethyl-3-pyrrolidin-3-yl-propionitrile dihydrochloride salt

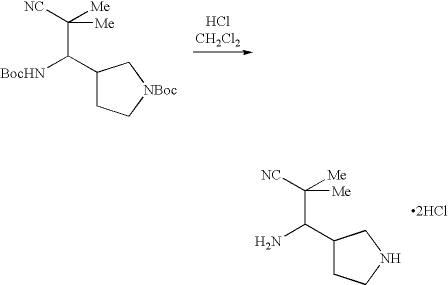

To a solution of 3-(1-tert-butoxycarbonylamino-2-cyano-2,2-dimethyl-ethyl)-pyrrolidine-1-carboxylic acid tert-butyl ester (1.52 g, 4.3 mmol) in CH₂Cl₂ (100 mL) at 0° C. was added HCl (21.5 mL of a 4 M solution in dioxane, 86 mmol). After 10 minutes, the reaction mixture was warmed to room temperature and stirred for 18 hours before it was concentrated under reduced pressure. The oily residue was taken up in water, extracted twice with CH₂Cl₂, and the aqueous phase concentrated under reduced pressure to give the title compound (704 mg, 73%). ¹H NMR □(D₂O) 3.68-3.82 (m, 2H), 3.52-3.63 (m, 1H), 3.17-3.45 (m, 2H), 2.86-3.12 (m, 1H), 2.46 (m, 1H), 1.89-2.10 (m, 1H), 1.60 (s, 1.5H), 1.59 (s, 1.5H), 1.57 (s, 1.5H), 1.56 (s, 1.511). LCMS (APCI⁺) 168 (100%, MH⁺).

### EXAMPLE 13

### Preparation of (±)-N-[1-azetidin-3-yl)-2-cyanoethyl]-2,2,2-trifluoroacetamide hydrochloride

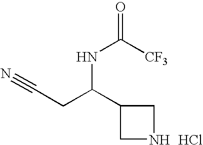

### A. Cis/trans-3-(1-Benzhydrylazetidin-3-yl)-acrylonitrile

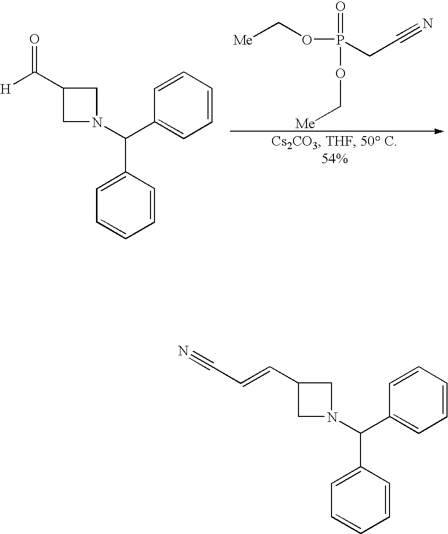

A solution of 1-benzhydrylazetidine-3-carbaldehyde (1.55 g, 6.17 mmol), diethyl(cyanomethyl)phosphonate (1.30 mL, 8.02 mmol), and cesium carbonate (2.61 g, 8.02 mmol) in tetrahydrofuran (30 mL) was heated at 50° C. for 2 hours. The solution was cooled to room temperature and diluted with ethyl acetate (100 mL). The solution was then washed with saturated aqueous ammonium chloride (20 mL). The organic layer was then dried (magnesium sulfate), filtered, and concentrated under reduced pressure. The resulting residue was purified via medium pressure liquid chromatography eluting with a gradient of hexanes:ethyl acetate (90:10 to 75:25) to deliver 913 mg (54%) of the title compound as a 1:1 mixture of cis/trans-isomers. The isomers were collected separately but later combined. cis-isomer: MS (APCI) (M+1)/Z 275.0; m.p.=117-120° C. trans-isomer: MS (APCI) (M+1/Z) 275.0; m.p.=108-110° C.

### B. (±)-3-Amino-3-(1-benzhydrylazetidin-3-yl)-propionitrile

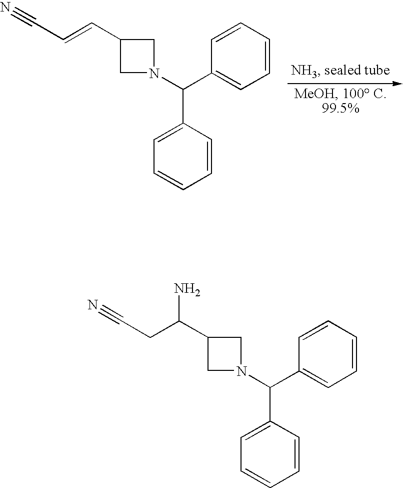

A saturated solution of ammonia in methanol (30 mL) was added to a 1:1 mixture of cis/trans-3-(1-benzhydrylazetidin-3-yl)-acrylonitrile (863 mg, 3.15 mmol). The resulting suspension was then heated in a sealed tube at 100° C. for 19 hours. After cooling to room temperature, the solution was concentrated under reduced pressure to deliver 912 mg (99.5%) of the title compound as an oil. MS (APCI) (M+1)/Z 292.1.

### C. (±)—N-[1-(1-benzhydryl-azetidin-3-yl)-2-cyanoethyl]-2,2,2-trifluoroacetamide

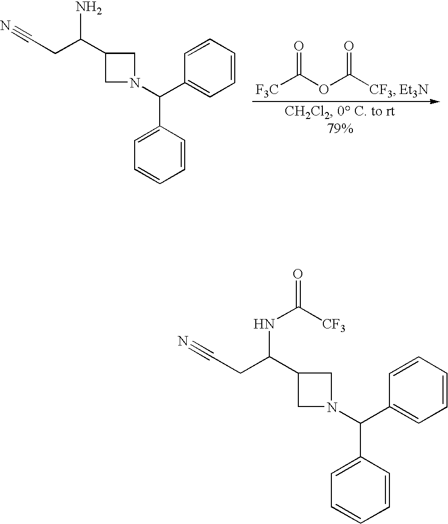

A solution of 3-amino-3-(1-benzhydrylazetidin-3-yl)-propionitrile (905 mg, 3.11 mmol) and triethylamine (1.30 mL, 9.32 mmol) in dichloromethane (30 mL) at 0° C. was treated with trifluoroacetic anhydride (0.659 mL, 4.67 mmol). The solution was then stirred at room temperature for 45 minutes. The solution was then cooled to 0° C., and water (5 mL) was added. The mixture was then further diluted with dichloromethane (50 mL) and water (15 mL). The layers were separated, and the organic layer washed with water (2×20 mL). The organic layer was then dried (magnesium sulfate), filtered, and concentrated under reduced pressure. The resulting residue was purified via medium pressure liquid chromatography eluting with a gradient of hexanes:ethyl acetate (75:25 to 55:45) to deliver 952 mg (79%) of the title compound. MS (APCI) (M+1)/Z 388.0.

### D. (±)-N-[1-azetidin-3-yl)-2-cyanoethyl]-2,2,2-trifluoroacetamide hydrochloride

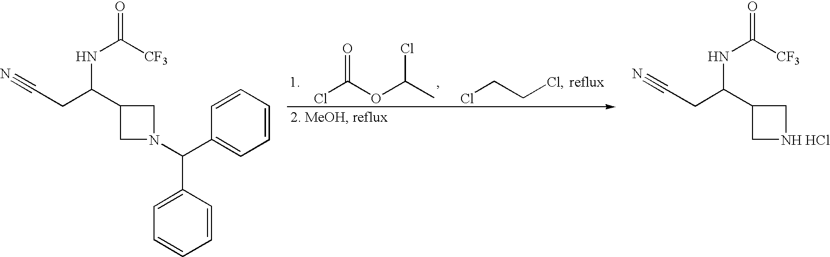

A solution of N-[1-(1-benzhydrylazetidin-3-yl)-2-cyanoethyl]-2,2,2-trifluoroacetamide (491 mg, 1.27 mmol) in dichloroethane (15 mL) was cooled to 0° C., whereupon, 1-chloroethyl chloroformate (0.410 mL, 3.80 mmol) was added. The resulting solution was heated at reflux for 2 hours. The solution was then concentrated under reduced pressure to deliver an oil. Methanol (15 mL) was added to the oil, and the resulting solution was heated at reflux for 2 hours. The solvent was removed under reduced pressure to deliver a thick yellow oil. The oil was triturated with hexanes several times, and the supernatant was discarded. The title compound was delivered as a yellow residue, 391 mg. MS (APCI) (M+1)/Z 222.0.

### EXAMPLE 14

### Preparation of (2-Cyano-1-pyrrolidin-3-yl-ethyl)-methyl-carbamic acid tert-butyl ester

### A. 3-(1-tert-Butoxycarbonylamino-2-cyano-ethyl)-pyrrolidine-1-carboxylic acid benzyl ester

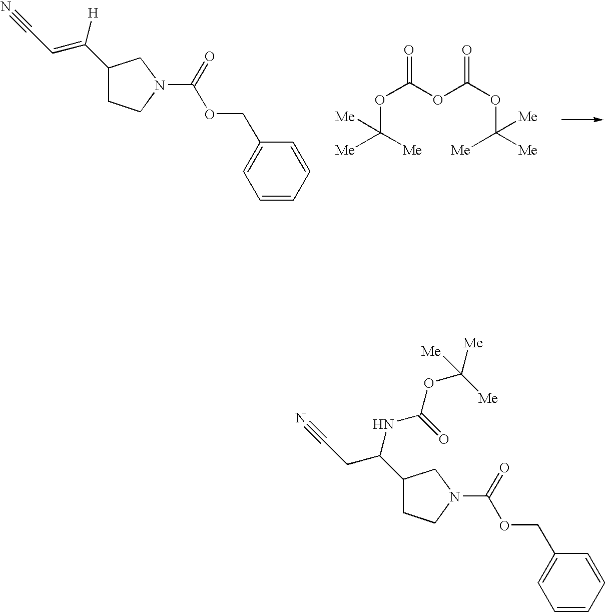

To a solution of 3-(2-cyano-vinyl)-pyrrolidine-1-carboxylic acid benzyl ester (4.40 g, 17.2 mmol) in absolute ethanol (50 mL) was added methyl amine (approximately 3 mL) and the solution was heated in a sealed reactor at 80° C. for 14 hours. The solution was concentrated in vacuo. The resulting amine was dissolved in THF (100 mL), Boc anhydride (5.62 g, 25.7 mmol) was added, and the solution was stirred at room temperature for 17 hours. The solution was conc. in vacuo. The residue was taken up in ethyl acetate (100 mL), washed with satd. aq. NH₄Cl (100 mL) and brine (100 mL), dried with MgSO₄ and concentrated in vacuo. The crude product was purified on a 120 g silica gel column eluted with 20 to 60% ethyl acetate in hexanes over 60 minutes at 50 mL/min to give 6.08 g of the title compound in multiple fractions (combined yield: 91%). MS (APCI+): m/z 288 (M+H-Boc). Diastereomer A (top spot) yield was 2.59 g (39%) and diastereomer B (bottom spot) yield was 2.82 g (42%). 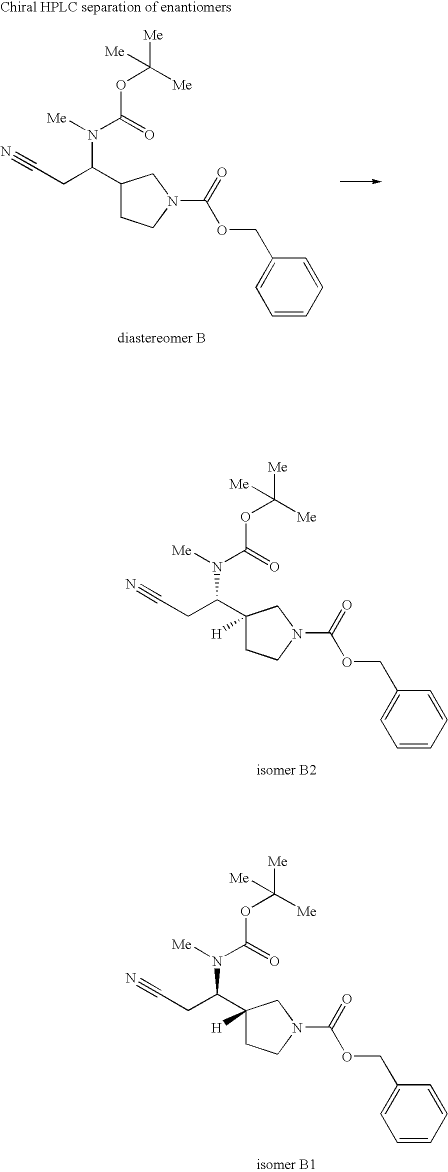
Diastereomer B (2.1 g) was separated by chiral HPLC using a ChiralPak AD column eluted with a methanol/ethanol gradient to give 0.87 g of isomer B1 (41%) and 0.53 g of isomer B2 (25%).

### B. (2-Cyano-1-pyrrolidin-3-yl-ethyl)-methyl-carbamic acid tert-butyl ester

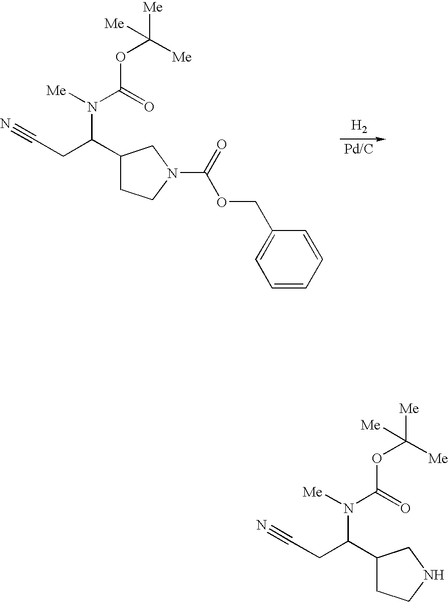

A solution of 3-(1-tert-butoxycarbonylamino-2-cyano-ethyl)-pyrrolidine-1-carboxylic acid benzyl ester (diastereomer B, 0.690 g, 1.78 mmol) in THF (50 mL) was hydrogenated with 10% Pd/C. The catalyst was removed by filtration and the filtrate was concentrated in vacuo to give 0.436 g of title compound (yield: 97%). MS (APCI+): m/z 254 (M+H).

### EXAMPLE 15

### Alternative Preparation of 3-(1-tert-Butoxycarbonylamino-2-cyano-ethyl)-pyrrolidine-1-carboxylic acid benzyl ester

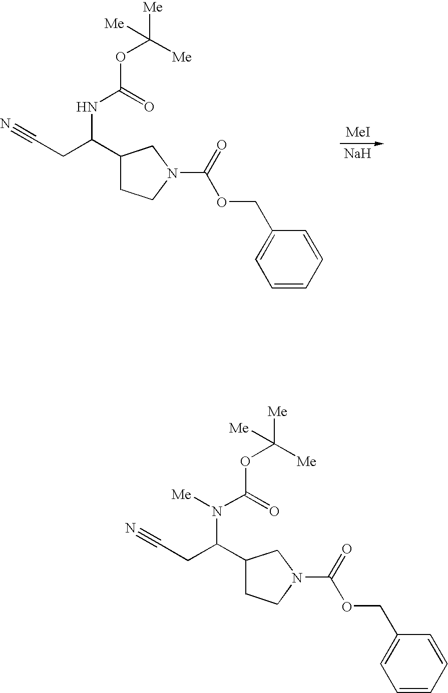

To a solution of 3-(1-tert-butoxycarbonylamino-2-cyano-ethyl)-pyrrolidine-1-carboxylic acid benzyl ester (isomer B2) (586 mg, 1.57 mmol) in anhydrous DMF (12 mL) was added NaH (60 wt %, 188 mg, 4.71 mmol) and the solution was stirred at room temperature for 1 hour. Methyl iodide (1.78 g, 12.5 mmol) was then added to the mixture and it was stirred at room temperature for 1 hour. The solution was poured into satd. aq. NH4Cl (80 mL) and extracted with diethyl ether (120 mL). The organics were washed with brine (50 mL), dried with MgSO₄ and concentrated in vacuo. The crude was run on a 10 g silical gel column eluted with 20 to 70% ethyl acetate in hexanes over 1 hour to give 0.44 g of the title compound (yield: 72%). MS (APCI+): m/z 288 (M+H-Boc).

### B. Coupling of Sidechain Precursors to Aminoquinazolinedione Cores

Coupling of the sidechain precursor to the quinazolinedione core precursor to provide the compounds of the present invention occurs as described in WO/02 102793, priority date Jun. 19, 2001 and WO/01 53273, priority date Oct. 18, 2000, and references cited therein, or as indicated below.

### EXAMPLE 14

### Preparation of 3-Amino-3-[1-(3-amino-1-cyclopropyl-6-fluoro-8-methoxy-2,4-dioxo-1,2,3,4-tetrahydro-quinazolin-7-yl)-pyrrolidin-3-yl]-propionitrile

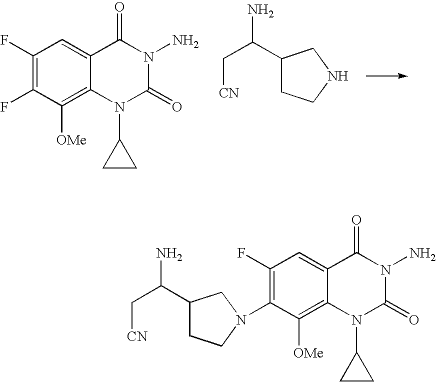

To a solution of 3-amino-3-pyrrolidin-3-yl-propionitrile (0.350 g, 2.51 mmol)) in DMSO (4 mL) was added 3-amino-1-cyclopropyl-6,7-difluoro-8-methoxy-1H-quinazoline-2,4-dione (0.475 g, 1.68 mmol) followed by 1,1,3,3-tetramethylguanidine (0.420 mL, 3.35 mmol). The reaction mixture was heated to 90° C. and stirred overnight. The reaction mixture was poured into water and extracted with ethyl acetate. The organic layer was dried over magnesium sulfate, filtered and concentrated. The crude residue was purified by flash column chromatography (0 to 4% methanol in dichloromethane) to afford 410 mg of the crude title compound. The product was dissolved in dichloromethane and HCl gas was bubbled in for 5 minutes. The mixture was concentrated, and the remaining solid was precipitated from hot ethanol. The mixture was filtered to give 310 mg (50% yield) of the title compound as the HCl salt. MS (APCI+): m/z 403 (M+H)⁺.

### EXAMPLE 15

### Preparation of 3-Amino-3-[1-(3-1-cyclopropyl-6-fluoro-8-methyl-2,4-dioxo-1,2,3,4-tetrahydro-quinazolin-7-yl)-pyrrolidin-3-yl]-propionitrile

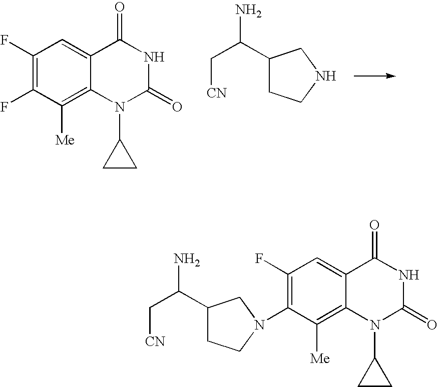

To a solution of 3-amino-3-pyrrolidin-3-yl-propionitrile (0.200 g, 1.44 mmol)) in DMSO (3 mL) was added 1-cyclopropyl-6,7-difluoro-8-methyl-1H-quinazoline-2,4-dione (0.242 g, 0.958 mmol) followed by 1,1,3,3-tetramethyl guanidine (0.180 mL, 1.44 mmol). The reaction mixture was heated to 90° C. and stirred overnight. The reaction mixture was poured into water and extracted with ethyl acetate. The organic layer was dried over magnesium sulfate, filtered and concentrated. The crude residue was purified by flash column chromatography (0 to 5% methanol in dichloromethane) to afford 87 mg of the crude product. The product was dissolved in dichloromethane and a saturated solution of HCl in dichloromethane (3 mL) was added. After stirring for 15 minutes, the mixture was concentrated and the remaining solid was precipitated from hot ethanol. The mixture was filtered to give 39 mg (11% yield) of the title compound as the HCl salt. MS (APCI+): m/z 370 (M+H)⁺.

### EXAMPLE 16

### Preparation of 3-Amino-3-[1-(3-1-cyclopropyl-6-fluoro-8-methyl-2,4-dioxo-1,2,3,4-tetrahydro-quinazolin-7-yl)-pyrrolidin-3-yl]-propionitrile

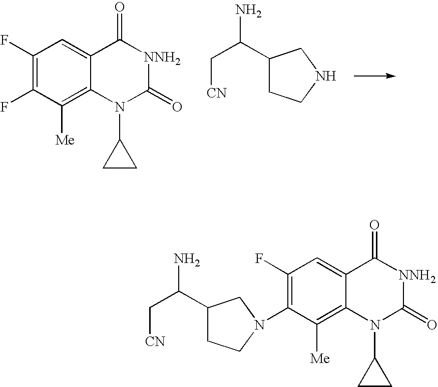

To a solution of 3-amino-3-pyrrolidin-3-yl-propionitrile (0.307 g, 2.21 mmol)) in DMSO (4 mL) was added 3-amino-1-cyclopropyl-6,7-difluoro-8-methyl-1H-quinazoline-2,4-dione (0.393 g, 1.47 mmol) followed by 1,1,3,3-tetramethylguanidine (0.369 mL, 2.94 mmol). The reaction mixture was heated to 90° C. and stirred overnight. The reaction mixture was poured into water and extracted with ethyl acetate. The organic layer was dried over magnesium sulfate, filtered and concentrated. The crude residue was purified by flash column chromatography (0 to 4% methanol in dichloromethane) to afford 210 mg (37%) of the title compound. MS (APCI+): m/z 387 (M+H)⁺.

### EXAMPLE 17

### Preparation of 3-[2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile

### A. [2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-yl]-carbamic acid tert-butyl ester

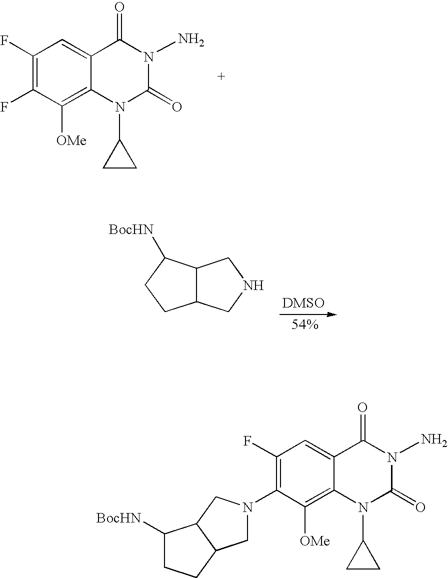

To a solution of 3-amino-1-cyclopropyl-6,7-difluoro-8-methoxy-1H-quinazoline-2,4-dione [WO 01/53273] (0.500 g, 1.77 mmol) in dimethylsulfoxide (4 mL) was added (octahydro-cyclopenta[c]pyrrol-4-yl)-carbamic acid tert-butyl ester [WO 96/39407] (1.2 g, 5.1 mmol), and the reaction mixture was heated at 110° C. After 5 hours, the mixture was cooled to room temperature and water was added. The resulting solid was filtered and purified on a 40M Biotage flash column (4:1 ethyl acetate/hexanes) to afford the title compound (0.470 g, 54%) as a white solid. MS: m/z 490.0 (M+H)⁺.

### B. 3-Amino-7-(4-amino-hexahydro-cyclopenta[c]pyrrol-2-yl)-1-cyclopropyl-6-fluoro-8-methoxy-1H-quinazoline-2,4-dione

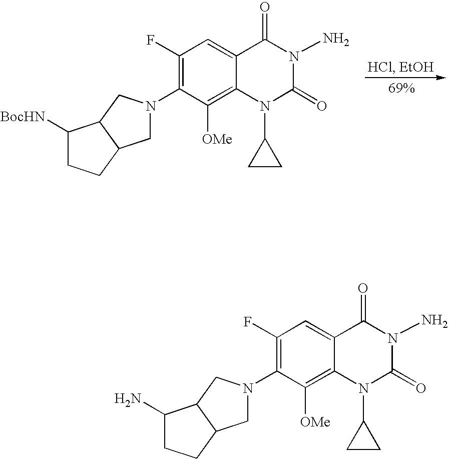

To a solution of [2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-yl]-carbamic acid tert-butyl ester (0.470 g, 0.96 mmol) in ethyl acetate (5 mL) was added 5 mL of saturated hydrochloric acid in ethanol, and the reaction mixture was allowed to stir for 5 hours. The solvent was removed in vacuo, and the product was recrystallized from 4 mL of 2:1 ethanol/isopropyl alcohol to afford the title compound (0.258 g, 69%); mp 250-252° C.

### C. Preparation of 3-[2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile

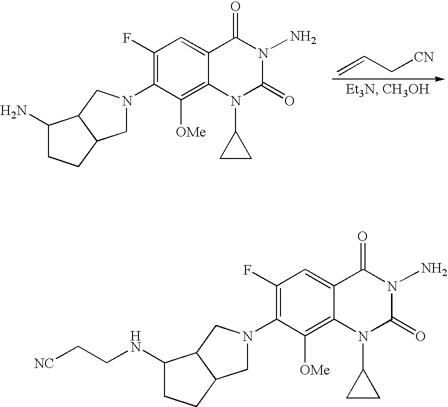

To a cooled (0° C.) solution of 3-amino-7-(4-amino-hexahydro-cyclopenta[c]pyrrol-2-yl)-1-cyclopropyl-6-fluoro-8-methoxy-1H-quinazoline-2,4-dione (0.125 g, 0.321 mmol) and triethylamine (89.5 μL, 0.642 mmol) in methanol (1 mL) was added acrylonitrile (23.2 μL, 0.353 mmol). The reaction mixture was allowed to warm to room temperature and stirred for 18 hours. The volatiles were removed in vacuo, and the resulting residue was purified on a 12 g Isco flash column (0:100 to 5:95 methanol/dichloromethane) to afford the title compound (88 mg, 62%). MS (APCI+): m/z 443.3 (M+H)⁺.

### EXAMPLE 18

### Preparation of 3-{[1-(3-Amino-1-cyclopropyl-6-fluoro-8-methyl-2,4-dioxo-1,2,3,4-tetrahydro-quinazolin-7-yl)-4-methyl-pyrrolidin-3-ylmethyl]-amino}-propionitrile

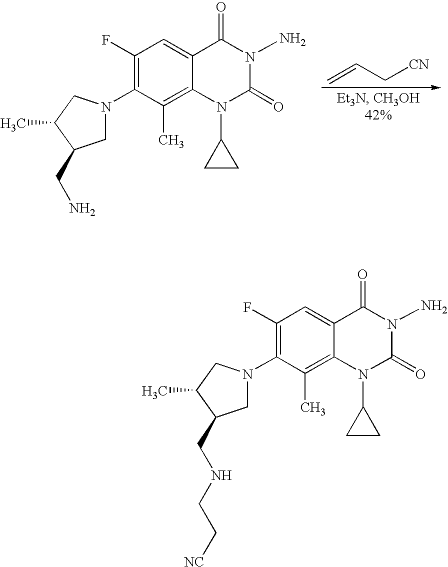

To a cooled (0° C.) solution of 3-amino-7-(trans-3-aminomethyl-4-methylpyrrolidin-1-yl)-1-cyclopropyl-6-fluoro-8-methyl-1H-quinazoline-2,4-dione hydrochloride [WO 01/53273] (0.022 g, 0.061 mmol) and triethylamine (17.0 μL, 0.122 mmol) in methanol (0.5 mL) was added acrylonitrile (4.4 μL, 0.067 mmol). The reaction mixture was allowed to warm to room temperature and stirred for 18 hours. The volatiles were removed in vacuo, and the resulting residue was purified via flash column (0:100 to 5:95 methanol/dichloromethane) to afford the title compound (10.5 mg, 42%) as a white solid. MS: m/z 415.0 (M+H)⁺.

### EXAMPLE 19

### Preparation of 3-({[1-(3-Amino-1-cyclopropyl-6-fluoro-8-methyl-2,4-dioxo-1,2,3,4-tetrahydro-quinazolin-7-yl)-pyrrolidin-3-yl]-oxazol-2-yl-methyl}-amino)-propionitrile

### A. 3-Amino-7-[3-(amino-oxazol-2-yl-methyl)-pyrrolidin-1-yl]-1-cyclopropyl-6-fluoro-8-methyl-1H-quinazoline-2,4-dione

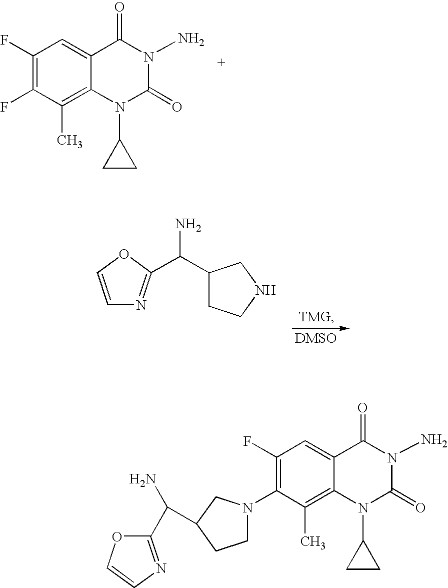

To a mixture of 3-amino-1-cyclopropyl-6,7-difluoro-8-methyl-1H-quinazoline-2,4-dione [WO 01/53273] (0.36 g, 2.2 mmol) and C-Oxazol-2-yl-C-pyrrolidin-3-yl-methylamine (0.41 g, 1.5 mmol) in dimethylsulfoxide (1.5 mL) was added 1,1,3,3-tetramethylguanidine (0.70 mL, 0.65 mmol). The reaction mixture was heated at 85° C. for 48 hours, cooled to the room temperature, treated with brine (20 mL), and extracted with ethyl acetate (2×20 mL). The combined organic extracts were washed with brine, dried over sodium sulfate, filtered, and concentrated in vacuo. The resulting residue was purified by flash chromatography (19:1 dichloromethane/methanol) to afford the title compound, which was used in the next step without further purification.

### B. {[1-(3-Amino-1-cyclopropyl-6-fluoro-8-methyl-2,4-dioxo-1,2,3,4-tetrahydro-quinazolin-7-yl)-pyrrolidin-3-yl]-oxazol-2-yl-methyl}-carbamic acid tert-butyl ester

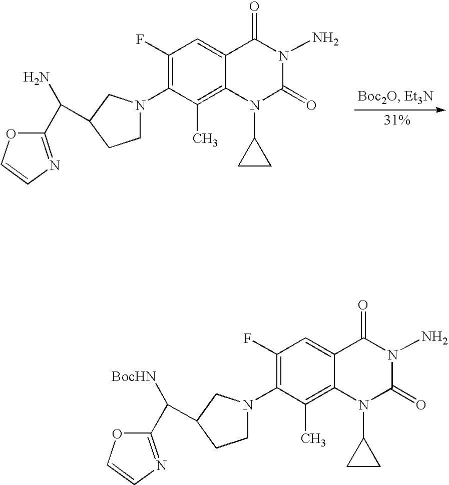

To the solution of 3-Amino-7-[3-(amino-oxazol-2-yl-methyl)-pyrrolidin-1-yl]-1-cyclopropyl-6-fluoro-8-methyl-1H-quinazoline-2,4-dione (0.2 g) in dichloromethane (15 mL) was added triethylamine (0.16 mL) and di-tert-butyl dicarbonate (0.14 g), and the mixture was stirred at room temperature for 18 hours. The solvent was removed in vacuo, and the residue was purified by flash chromatography (9:1 ethyl acetate/methanol) to afford the title compound (0.24 g, 31%). ¹H NMR (400 MHz, CDCl₃): δ 7.74-7.55 (m, 2H), 7.11 (d, 1H), 5.42-4.98 (m, 4H), 3.61-3.25 (m, 5H), 2.94-2.77 (m, 1H), 2.39 (d, 3H), 2.21-1.79 (m, 2H), 1.41 (s, 9H), 1.21-0.90 (m, 2H), 0.69-0.52 (m, 2H).

### C. 3-Amino-7-[3-(amino-oxazol-2-yl-methyl)-pyrrolidin-1-yl-1-cyclopropyl-6-fluoro-8-methyl-1H-quinazoline-2,4-dione

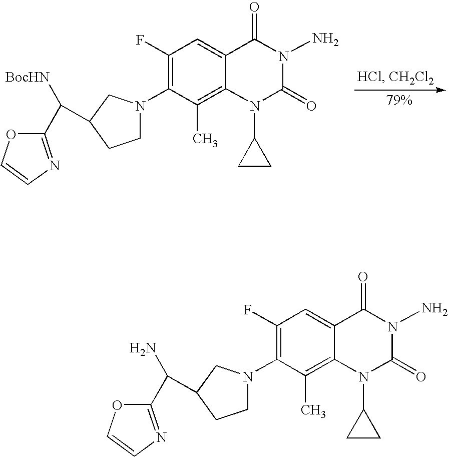

To a solution of {[1-(3-Amino-1-cyclopropyl-6-fluoro-8-methyl-2,4-dioxo-1,2,3,4-tetrahydro-quinazolin-7-yl)-pyrrolidin-3-yl]-oxazol-2-yl-methyl}-carbamic acid tert-butyl ester (0.24 g, 0.47 mmol) in dichloromethane (5 mL) was added 15 mL of saturated hydrogen chloride gas in diethyl ether, and the reaction mixture was stirred for 2 hours. The solvent was removed in vacuo to afford a white solid which was triturated with diethyl ether to afford the title compound (0.17 g, 79%). MS: m/z 414.9 (M+H)⁺.

### D. 3-({[1-(3-Amino-1-cyclopropyl-6-fluoro-8-methyl-2,4-dioxo-1,2,3,4-tetrahydro-quinazolin-7-yl)-pyrrolidin-3-yl]-oxazol-2-yl-methyl}-amino)-propionitrile

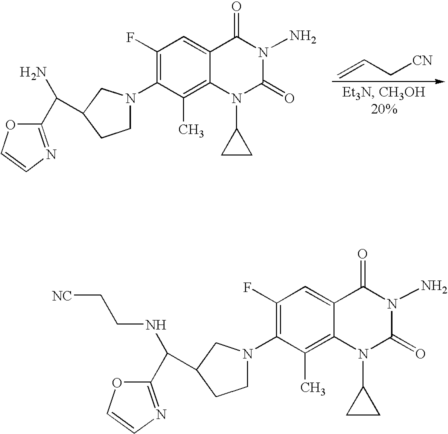

To a cooled (0° C.) solution of 3-Amino-7-[3-(amino-oxazol-2-yl-methyl)-pyrrolidin-1-yl]-1-cyclopropyl-6-fluoro-8-methyl-1H-quinazoline-2,4-dione (0.083 g, 0.20 mmol) and triethylamine (55.8 μL, 0.400 mmol) in methanol (1 mL) was added acrylonitrile (43.5 μL, 0.661 mmol). The reaction mixture was allowed to warm to room temperature and stirred for 72 hours. The volatiles were removed in vacuo, and the resulting residue was purified on a 12 g Isco flash column (0:100 to 5:95 methanol/dichloromethane) to afford the title compound (19.1 mg, 20%) as a 60:40 mixture of diastereomers as a yellow solid. MS: m/z 468.1 (M+H)⁺.

### EXAMPLE 20

### Preparation of 3-{[1-(3-Amino-1-cyclopropyl-6-fluoro-8-methyl-2,4-dioxo-1,2,3,4-tetrahydro-quinazolin-7-yl)-4-fluoro-pyrrolidin-3-ylmethyl]-amino}-propionitrile

### A. [1-(3-Amino-1-cyclopropyl-6-fluoro-8-methyl-2,4-dioxo-1,2,3,4-tetrahydro-quinazolin-7-yl)-4-fluoro-pyrrolidin-3-ylmethyl]-carbamic acid tert-butyl ester

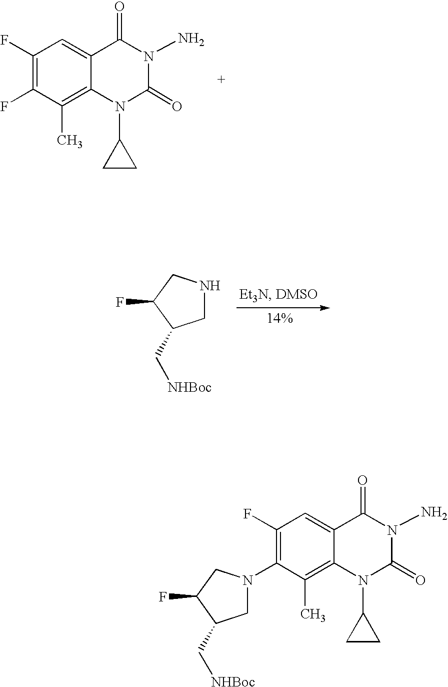

A mixture of 3-amino-1-cyclopropyl-6,7-difluoro-8-methyl-1H-quinazoline-2,4-dione [WO 01/53273] (0.37 g, 1.38 mmol), trans-(4-fluoropyrrolidine-3-ylmethyl)carbamic acid tert-butyl ester [WO 01/53273] (0.60 g, 2.7 mmol), and triethylamine (0.77 mL, 5.5 mmol) in dimethyl sulfoxide (4 mL) was heated in a sealed tube at 120° C. for 2 days. The mixture was cooled, diluted with water, and extracted with ethyl acetate. The combined organic extracts were dried over sodium sulfate, filtered, and concentrated in vacuo. The residue was purified by preparative thin layer chromatography (8:92 methanol/dichloromethane) to afford the title compound (0.09 g, 14%) as a thick oil. ¹H NMR (400 MHz, CDCl₃) δ 8.00 (br s, 1H), 7.58 (d, 1H), 5.18 (br s, 2H), 5.07 (d, 1H), 3.96-3.83 (m, 1H), 3.58-3.39 (m, 2H), 3.35-3.09 (m, 2H), 3.04-2.82 (m, 1H), 2.80-2.40 (m, 5H), 1.51 (s, 9H), 1.20 (m, 2H), 0.68 (m, 2H).

### B. 3-Amino-7-(3-aminomethyl-4-fluoro-pyrrolidin-1-yl)-1-cyclopropyl-6-fluoro-8-methyl-1H-quinazoline-2,4-dione

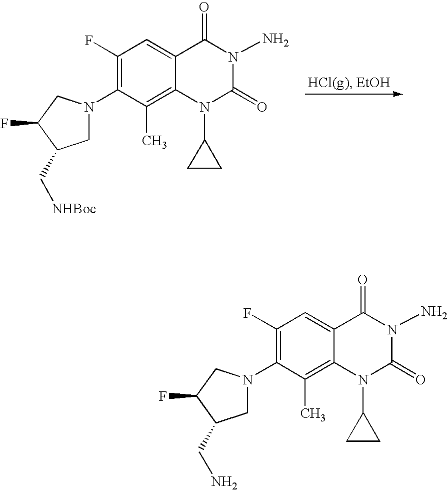

Hydrogen chloride gas was bubbled into a cooled (0° C.) solution of [1-(3-Amino-1-cyclopropyl-6-fluoro-8-methyl-2,4-dioxo-1,2,3,4-tetrahydro-quinazolin-7-yl)-4-fluoro-pyrrolidin-3-ylmethyl)-carbamic acid tert-butyl ester (0.090 g, 0.19 mmol) in anhydrous ethanol (4 mL) for 10 minutes. The suspension was slowly warmed to room temperature and stirred for 16 hours. The solvent was evaporated in vacuo, washed with diethyl ether, and dried to provide the title compound (0.76 g); mp 170-172° C.

### C. 3-{[1-(3-Amino-1-cyclopropyl-6-fluoro-8-methyl-2,4-dioxo-1,2,3,4-tetrahydro-quinazolin-7-yl)-4-fluoro-pyrrolidin-3-ylmethyl]-amino}-propionitrile

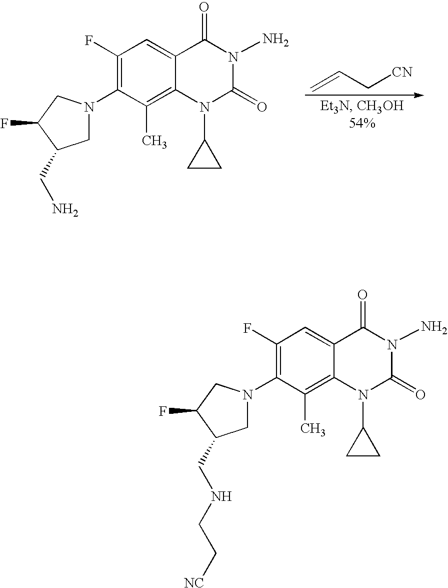

To a cooled (0° C.) solution of 3-amino-7-(3-aminomethyl-4-fluoro-pyrrolidin-1-yl)-1-cyclopropyl-6-fluoro-8-methyl-1H-quinazoline-2,4-dione (0.037 g, 0.101 mmol) and triethylamine (28.2 μL, 0.202 mmol) in methanol (1 mL) was added acrylonitrile (7.3 μL, 0.11 mmol). The reaction mixture was allowed to warm to room temperature and stirred for 65 hours. The volatiles were removed in vacuo, and the resulting residue was purified on a 12 g Isco flash column (0:100 to 5:95 methanol/dichloromethane) to afford the title compound (23.0 mg, 54%) as a yellow solid. MS: m/z 419.2 (M+H)⁺.

### EXAMPLE 21

### Preparation of 3-Amino-3-[1-(1-cyclopropyl-6-fluoro-8-methyl-2,4-dioxo-1,2,3,4-tetrahydro-quinazolin-7-yl)-pyrrolidin-3-yl]-propionitrile

### A. {2-Cyano-1-[1-(1-cyclopropyl-6-fluoro-8-methyl-2,4-dioxo-1,2,3,4-tetrahydro-quinazolin-7-yl)-pyrrolidin-3-yl]-ethyl}-carbamic acid tert-butyl ester

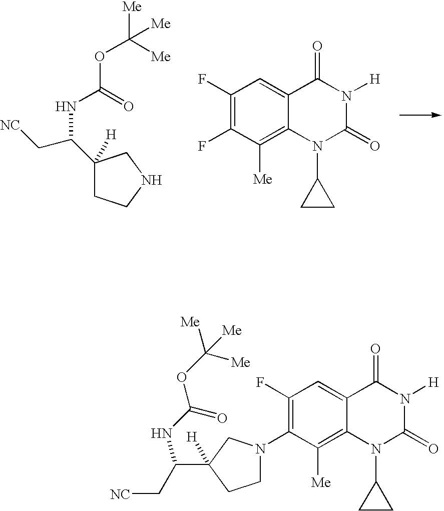

To a solution of (2-cyano-1-pyrrolidin-3-yl-ethyl)-carbamic acid tert-butyl ester (0.340 g, 1.4 mmol)) in DMSO (4 mL) was added 1-cyclopropyl-6,7-difluoro-8-methyl-1H-quinazoline-2,4-dione (0.240 g, 0.96 mmol) followed by 1,1,3,3-tetramethylguanidine (0.180 mL, 1.4 mmol). The reaction mixture was heated to 90° C. and stirred overnight. The reaction mixture was poured into water and extracted with ethyl acetate. The organic layer was dried over magnesium sulfate, filtered and concentrated. The crude residue was purified by flash column chromatography (0 to 70% ethyl acetate in hexanes) to afford 130 mg (29%) of the title compound. MS (APCI+): m/e 472 (M+H)⁺.

### B. 3-Amino-3-[1-(1-cyclopropyl-6-fluoro-8-methyl-2,4-dioxo-1,2,3,4-tetrahydro-quinazolin-7-yl)-pyrrolidin-3-yl]-propionitrile

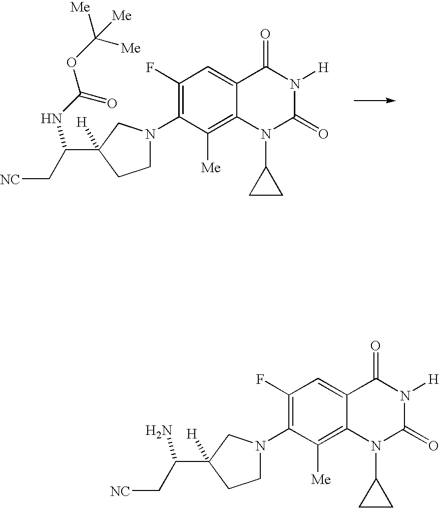

To a solution of {2-Cyano-1-[1-(1-cyclopropyl-6-fluoro-8-methyl-2,4-dioxo-1,2,3,4-tetrahydro-quinazolin-7-yl)-pyrrolidin-3-yl]-ethyl}-carbamic acid tert-butyl ester (0.13 g, 0.28 mmol) in dichloromethane (10 mL) was bubbled in HCl gas for 2 minutes. The reaction mixture stirred for 3 hours and was concentrated. 1N NaOH solution was added and the mixture was concentrated. The crude residue was purified by flash chromatography (0-10% methanol in dichloromethane) to afford 50 mg (50%) of the crude product. The mixture was dissolved in ethanol (5 mL) and 10% HCl in ethanol was added. The mixture was concentrated to give the title compound as the HCl salt. MS (APCI+): m/e 372 (M+H)+.

### EXAMPLE 22

### Preparation of 3-{1-[1-(3-Amino-1-cyclopropyl-6-fluoro-8-methoxy-2,4-dioxo-1,2,3,4-tetrahydro-quinazolin-7-yl)-pyrrolidin-3-yl]-ethylamino}-propionitrile

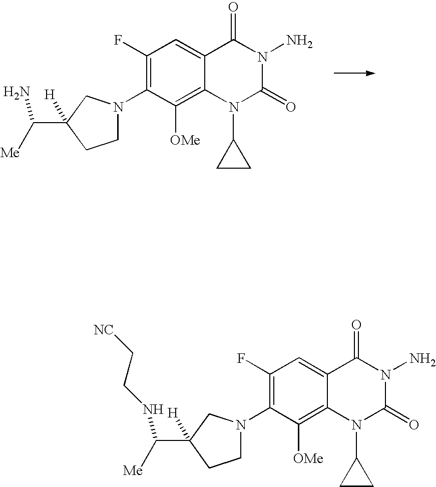

To a solution of 3-Amino-7-[3-(1-amino-ethyl)-pyrrolidin-1-yl]-1-cyclopropyl-6-fluoro-8-methoxy-1H-quinazoline-2,4-dione (0.31 g, 0.81 mmol) in methanol (4 mL) was added triethylamine (0.23 mL, 1.6 mmol) followed by acrylonitrile (0.064 mL, 0.98 mmol). The reaction stirred at room temperature overnight. An additional 0.042 mL of acrylonitrile was added and the mixture stirred overnight again. The reaction mixture was concentrated and the crude residue purified by flash column chromatography (0 to 5% methanol in dichloromethane) to afford the title compound as the free amine, which was diluted with dichloromethane. HCl gas was bubbled in for 30 sec. The mixture was concentrated to afford 165 mg (47%) of the title compound as the HCl salt. MS (APCI+): m/e 431 (M+H)⁺.

### EXAMPLE 23

### Preparation of [1-(3-Amino-1-cyclopropyl-6-fluoro-8-methyl-2,4-dioxo-1,2,3,4-tetrahydro-quinazolin-7-yl)-pyrrolidin-3-yl]-acetonitrile

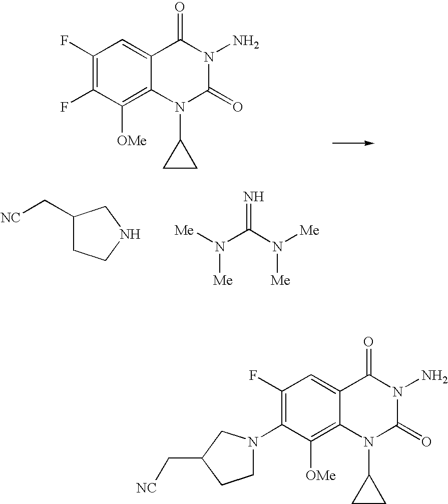

To a solution of 3-amino-1-cyclopropyl-6,7-difluoro-8-methyl-1H-quinazoline-2,4-dione (0.35 g, 1.31 mmol) in DMSO (3 mL) were added pyrrolidin-3-yl-acetonitrile (0.21 g, 1.97 mmol) and 1,1,3,3-tetramethylguanidine (0.30 g, 2.62 mmol). The reaction mixture was heated to 90° C. After 20 hours, the reaction mixture was poured into saturated sodium bicarbonate solution and extracted with ethyl acetate. The organic layer was dried over MgSO₄, filtered, and filtrate concentrated. The crude residue was purified by flash column chromatography (50% ethyl acetate in hexanes to 100% ethyl acetate) to afford 110 mg of the title compound (23% yield). MS (APCI+): m/z 358 (M+H)⁺.

### EXAMPLE 24

### Preparation of 3-[1-(3-Amino-1-cyclopropyl-6-fluoro-8-methyl-2,4-dioxo-1,2,3,4-tetrahydro-quinazolin-7-yl)-pyrrolidin-3-yl]-3-methylamino-propionitrile

### A. {1-[1-(3-Amino-1-cyclopropyl-6-fluoro-8-methyl-2,4-dioxo-1,2,3,4-tetrahydro-quinazolin-7-yl)-pyrrolidin-3-yl]-2-cyano-ethyl}-carbamic acid tert-butyl ester

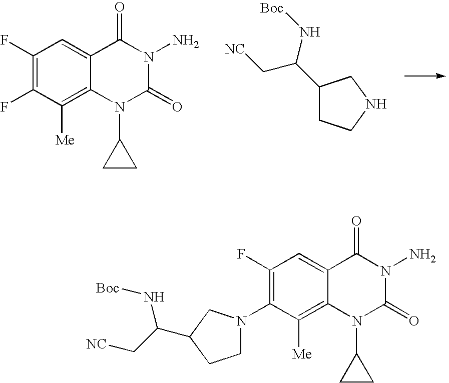

To a solution of 3-amino-1-cyclopropyl-6,7-difluoro-8-methyl-1H-quinazoline-2,4-dione (0.31 g, 1.16 mmol) in DMSO (2 mL) were added (2-cyano-1-pyrrolidin-3-yl-ethyl)-carbamic acid tert-butyl ester (0.33 g, 1.39 mmol) and tetramethylguanidine (0.27 g, 2.32 mmol). The reaction mixture was heated to 90° C. After 20 hours, the reaction mixture was poured into saturated sodium bicarbonate solution and extracted with ethyl acetate. The organic layer was dried over MgSO₄, filtered, and filtrate concentrated. The crude residue was purified by flash column chromatography (50% ethyl acetate in hexanes to 100% ethyl acetate over 50 minutes) to afford 133 mg of the title compound (24%). MS (APCI+): m/z 487 (M+H)⁺.

### B. 3-[1-(3-Amino-1-cyclopropyl-6-fluoro-8-methyl-2,4-dioxo-1,2,3,4-tetrahydro-quinazolin-7-yl)-pyrrolidin-3-yl]-3-methylamino-propionitrile

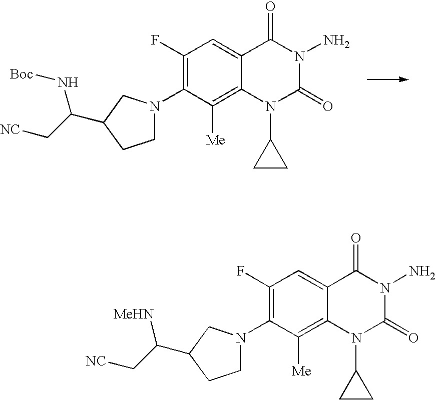

To a solution of {1-[1-(3-Amino-1-cyclopropyl-6-fluoro-8-methyl-2,4-dioxo-1,2,3,4-tetrahydro-quinazolin-7-yl)-pyrrolidin-3-yl]-2-cyano-ethyl}-carbamic acid tert-butyl ester (0.13 g, 0.27 mmol) in dichloromethane (5 mL) was added HCl (2M in ether, 2 mL, 4 mmol). After 5 hours, the precipitate formed was filtered, washed with hexanes and dried under vacuo to give 0.11 g of title compound as the HCl salt (97%).

### EXAMPLE 25

### Preparation of 3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-2,2-dimethyl-propionitrile

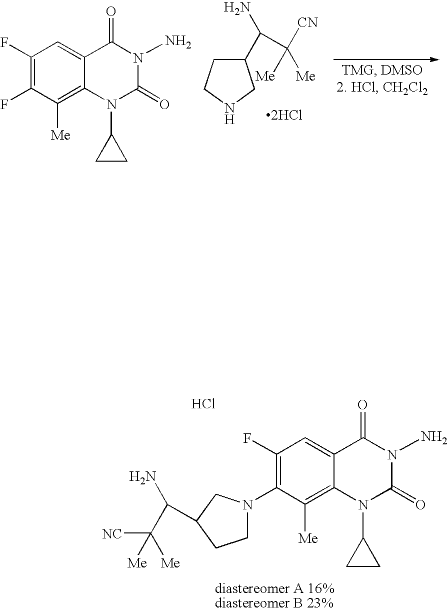

A solution of 3-amino-2,2-dimethyl-3-pyrrolidin-3-yl-propionitrile dihydrochloride salt (150 mg, 0.625 mmol), 3-amino-1-cyclopropyl-6,7-difluoro-8-methyl-1H-quinazoline-2,4-dione (133 mg, 0.50 mmol), 1,1,3,3-tetramethylguanidine (0.25 mL, 2.0 mmol) in DMSO (0.5 mL) was heated to 100° C. for 17 hours. After cooling to room temperature, the reaction mixture was subjected to preparative HPLC purification (Synergi Polar RP column, 0.8 mL/min, gradient NH₄⁺HCO₂⁻ pH 3.45:90% aqueous MeCN 90:10 to 1:1) to give firstly diastereomer A followed by diastereomer B. Each diastereomer was further purified by chromatography (CH₂Cl₂:MeOH:conc. NH₃ 95:5:0.5). The diastereomers were separately taken up in HCl (2 mL of a 1.25 mol L⁻¹ solution in MeOH), and concentrated under reduced pressure to give diastereomer A (35 mg, 16%). HPLC 97% (99.5:0.5 mixture of diastereomers). ¹H NMR □(D₂O) 7.53 (d, 1H), 4.76 (m, 1H), 3.63-3.80 (m, 3H), 3.44-3.54 (m, 2H), 2.89 (m, 1H), 2.45 (s, 3H), 2.35 (m, 1H), 1.98 (m, 1H), 1.61 (s, 3H), 1.58 (s, 3H), 1.15 (m, 2H), 0.64 (m, 2H). LCMS (APCI⁺) 415 (100%, MH⁺). HRMS (FAB⁺) Calc. for C₂₁H₂₈FN₆O₂ 415.22578. Found 415.22696; and diastereomer B (52 mg, 23%). HPLC 93% (99:1 mixture of diastereomers). 1H NMR □(D₂O) 7.54 (d, 1H), 4.76 (m, 1H), 3.65-3.76 (m, 3H), 3.42-3.51 (m, 2H), 2.95 (m, 1H), 2.46 (s, 3H), 2.39 (m, 1H), 2.02 (m, 1H), 1.62 (s, 3H), 1.59 (s, 3H), 1.15 (m, 2H), 0.64 (m, 2H). LCMS (APCI⁺) 415 (100%, MH⁺). HRMS (FAB⁺) Calc. for C₂₁H₂₈FN₆O₂ 415.22578. Found 415.22697.

### EXAMPLE 26

### Preparation of 3-{[1-(3-Amino-1-cyclopropyl-6-fluoro-8-methoxy-2,4-dioxo-1,2,3,4-tetrahydro-quinazolin-7-yl)-4-fluoro-pyrrolidin-3-ylmethyl]-amino}-propionitrile

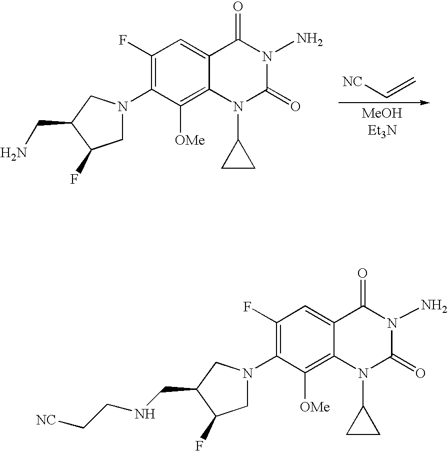

The 3-Amino-7-(3-aminomethyl-4-fluoro-pyrrolidin-1-yl)-1-cyclopropyl-6-fluoro-8-methoxy-1H-quinazoline-2,4-dione (WO2001053273A1) (0.101 g, 0.233 mmol) was taken up in methanol and charged with acrylonitrile (0.1 mL). The solution was charged with 1 equivalent of triethylamine. After 2 hours the solvent was removed in vacuo. The residue was taken up in dichloromethane and charged with 2N HCl/ether until cloudy. The resulting precipitate was collected via filtration and washed with diethyl ether leaving 62 mg of the title compound as the HCl salt (54% yield) MS (APCI+) m/z 435 (M+H)⁺.

### D. Pharmaceutical Formulations

### EXAMPLE 27

The following illustrates representative pharmaceutical dosage forms, containing a compound of Formula I (“Invention Compound”), for therapeutic or prophylactic use in humans.

(i)

| Tablet | mg/tablet |
|---|---|
| ‘Invention Compound’ | 25.0 |
| Lactose | 50.0 |
| Corn Starch (for mix) | 10.0 |
| Corn Starch (paste) | 10.0 |
| Magnesium Stearate (1%) | 3.0 |
| | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

(ii)

| Tablet | mg/capsule |
|---|---|
| ‘Invention Compound | 10.0 |
| Colloidal Silicon Dioxide | 1.5 |
| Lactose | 465.5 |
| Pregelatinized Starch | 120.0 |
| Magnesium Stearate (1%) | 3.0 |
| | 600.0 |

(iii)

| Preparation for | | |
|---|---|---|
| Oral Solution | Amount | |
| ‘Invention Compound’ | 400 | mg |
| Sorbitol Solution (70% N.F.) | 40 | mL |
| Sodium Benzoate | 20 | mg |
| Saccharin | 5 | mg |
| Cherry Flavor | 20 | mg |
| Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

(v)

| Injection 1 (1 mg/mL) | Amount |
|---|---|
| ‘Invention Compound’ | 1.0 |
| Dibasic Sodium Phosphate | 12.0 |
| Monobasic Sodium Phosphate | 0.7 |
| Sodium Chloride | 4.5 |
| N Sodium hydroxide solution | q.s. |
| (pH adjustment to 7.0-7.5) | |
| Water for injection | q.s. ad 1 mL |

(vi)

| Injection 2 (10 mg/mL) | Amount |
|---|---|
| ‘Invention Compound’ | 10.0 |
| Dibasic Sodium Phosphate | 1.1 |
| Monobasic Sodium Phosphate | 0.3 |
| Polyethylene glyco 400 | 200.0 |
| N hydrochloric acid solution | q.s. |
| (pH adjustment to 7.0-7.5) | |
| Water for injection | q.s. ad 1 mL |

(vii)

| Injection 2 (10 mg/mL) | Amount |
|---|---|
| ‘Invention Compound’ | 20.0 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of formula I: 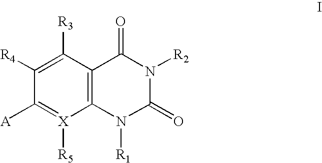
or a pharmaceutically acceptable salt thereof, wherein:
X is N or C, provided that when X is N, R₅ is absent at that position;
R₁ is (C₁-C₆)alkyl,
halo(C₁-C₆)alkyl,
(C₃-C₆)Cycloalkyl,
halo(C₃-C₆)cycloalkyl
aryl, and
heteroaryl;
CH₂(C₃-C₆)cycloalkyl;
R₂ is H,
NH₂, 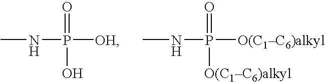
NH(C₁-C₆)alkyl,
NH(C₃-C₆)cycloalkyl,
NH-aryl,
NH-heteroaryl,
NHSO₂—(C₁-C₆)alkyl,
NHSO₂-aryl,
NHSO₂-heteroaryl, 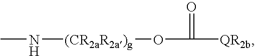
wherein g is an integer from 1 to 10, Q is O, NH, or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 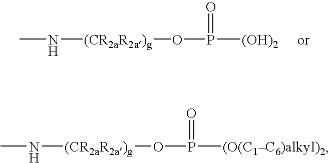
wherein R₂ₐ and R_{2a′} are as defined above, 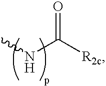
wherein [symbol] indicates the point of attachment, p is 0 or 1, and R_{2c} is H,
(C₁-C₆)alkyl,
O(C₁-C₆)alkyl,
(C₃-C₇)cycloalkyl,
aryl,
heterocyclo,
heteroaryl, or 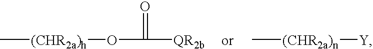
wherein R₂ₐ, R_{2b}, and Q, are as defined above, and h and n are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 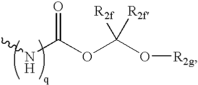
wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
(C₁-C₆)alkyl,
(C₃-C₇)cycloalkyl,
aryl, or
heterocyclo, or
heteroaryl;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy
nitrile; or
R₁ and R₅ taken together with the carbons to which they are attached form a substituted or unsubstituted 5- or 6-membered substituted or unsubstituted ring containing 0, 1, or 2 heteroatoms selected from O, S, SO, SO₂, or NRₓ, wherein Rₓ is H or (C₁-C₆)alkyl; and
A is 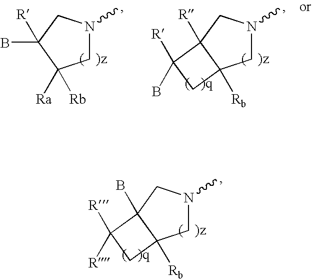
wherein z is 0, 1, or 2 and q is 0, 1, 2, or 3;
Rₐ and R_{b} are each independently H, (C₁-C₆)alkyl, (C₁-C₆)alkoxy, halo(C₁-C₆)alkyl, halo, or Rₐ and R_{b} taken together with the carbon to which they are attached form C═O, C═NO(C₁-C₆)alkyl, or a 3, 4, 5 or 6-membered substituted or unsubstituted ring;
R′, R″, R′″, and R″″ are each independently H,
(C₁-C₆)alkyl,
—O(C₁-C₆)alkyl,
halo(C₁-C₆)alkyl,
aryl, or
heteroaryl;
and B is 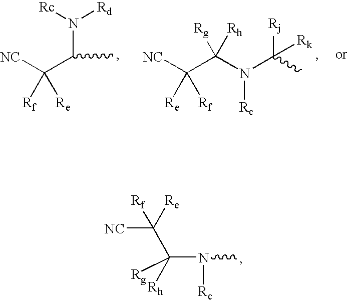
provided that when B is 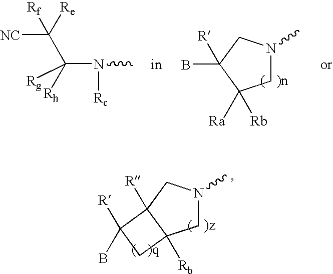
R′ is not —O(C₁-C₆)alkyl, and wherein “ [symbol] ” indicates the point of attachment;
R_{c} and R_{d} are each independently H,
(C₆-C₆)alkylnitrile; 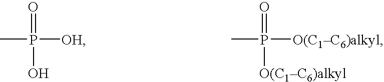
(C₁-C₆)alkyl,
(C₃-C₆)cycloalkyl,
heteroaryl,
SO₂—(C₁-C₆)alkyl,
SO₂-aryl,
SO₂-heteroaryl, 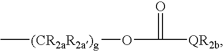
wherein g is an integer from 1 to 10, Q is as defined above, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 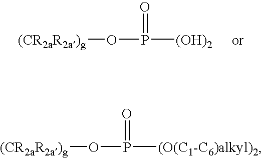
wherein R₂ₐ and R_{2a′} are as defined above, 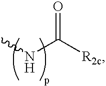
wherein [symbol] indicates the point of attachment, p is 0 or 1, and R_{2c} is H,
(C₁-C₆)alkyl,
O(C₁-C₆)alkyl,
(C₃-C₇)cycloalkyl,
aryl,
heterocyclo,
heteroaryl, or 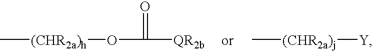
wherein R₂ₐ, R_{2b}, and Q are as defined above, and h and j are each independently integers from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 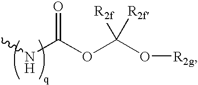
wherein q is 0 or 1, R_{2f} and R_{2f′} are each independently H, (C₁-C₆)alkyl, aryl, or heteroaryl, or taken together with the carbon to which they are attached form a 3, 4, 5, or 6 membered substituted or unsubstituted ring, and R_{2g} is
(C₁-C₆)alkyl,
(C₃-C₇)cycloalkyl,
aryl, or
heterocyclo, or
heteroaryl;
Rₑ , and R_{f} are each independently X, C₁-C₆ alkyl, haloalkyl, halo, or Rₑ and R_{f} taken together with the carbon to which they are attached form a 3, 4, 5 or 6-membered substituted or unsubstituted ring;
R_{g} and Rₕ are each independently H, C₁-C₆ alkyl, haloalkyl, or taken together with the carbon to which they are attached to form a 3, 4, 5 or 6-membered substituted or unsubstituted ring; and
Rⱼ and Rₖ, are each independently H, (C₁-C₆)alkyl, haloalkyl, (C₁-C₆)alkyl-NR_{c}R_{d}, (C₁-C₆)alkyl-OR_{c}, aryl, heteroaryl, heterocycle, 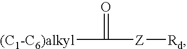
wherein Z is O or NR_{c}, or Rⱼ and Rₖ taken together with the carbon to which they are attached to form a 3, 4, 5 or 6-membered substituted or unsubstituted ring.

2. The compound of claim 1, wherein
X is C or N;
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy,
difluoromethoxy, or trifluoromethoxy when X is C.

3. The compound of claim 2, wherein
X is C or N;
R₁ is cyclopropyl or fluorocyclopropyl;
R₃ is H or NH₂;
R₄ is H or F;
R₅ is halo, methyl, or methoxy.

4. The compound of claim 1, wherein R₁, R₃, and R₅ are as provided in the following structures, and R₂ is NH₂ or H, and R₄ is H or F:

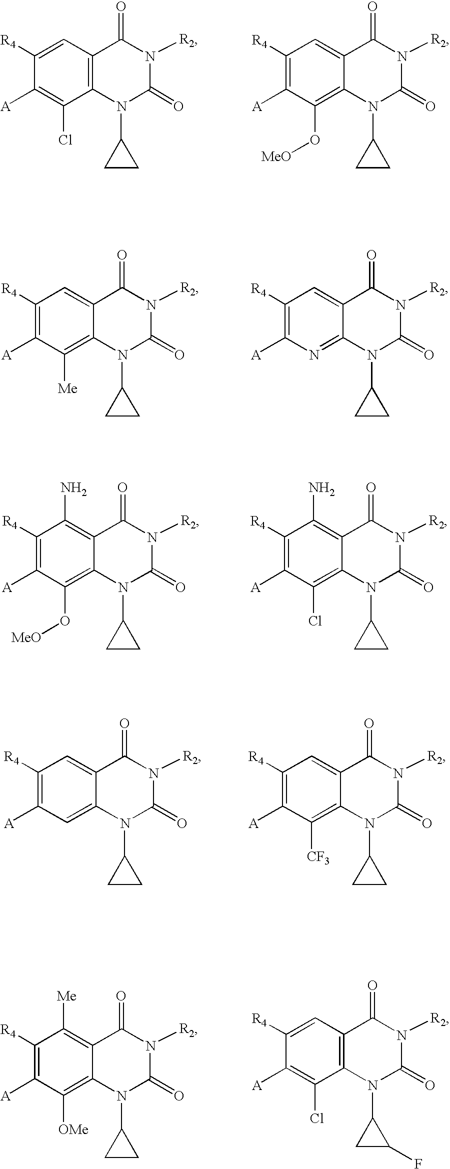

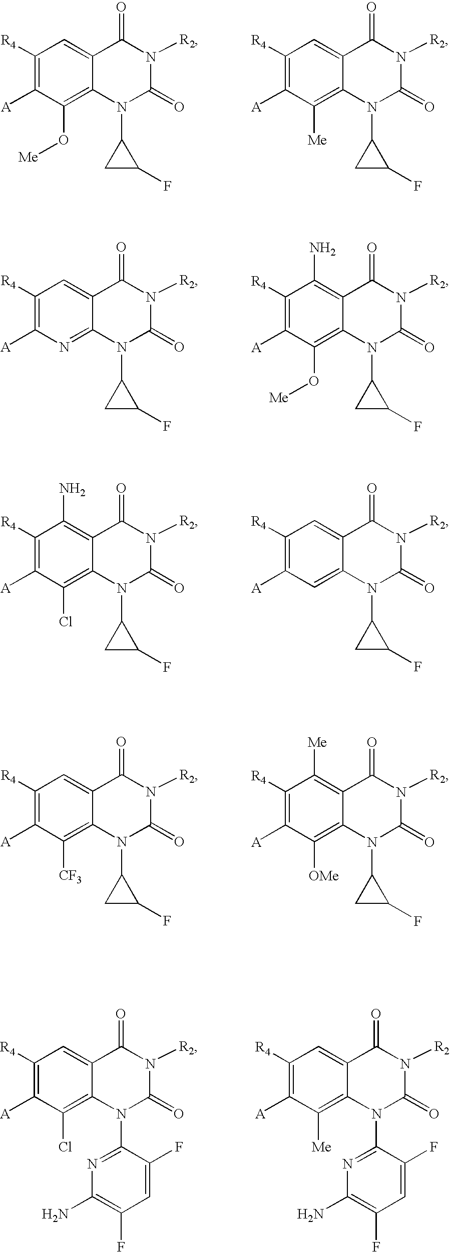

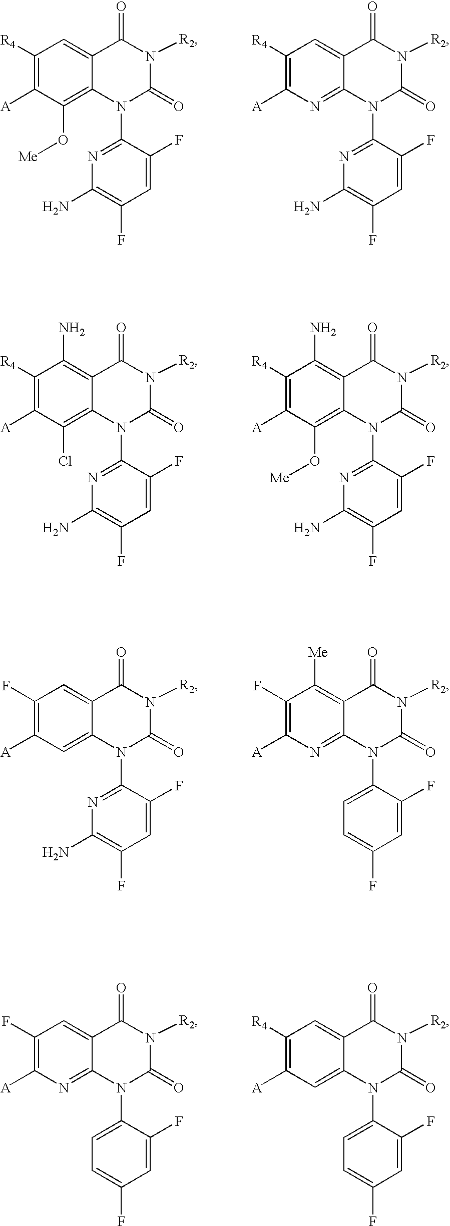

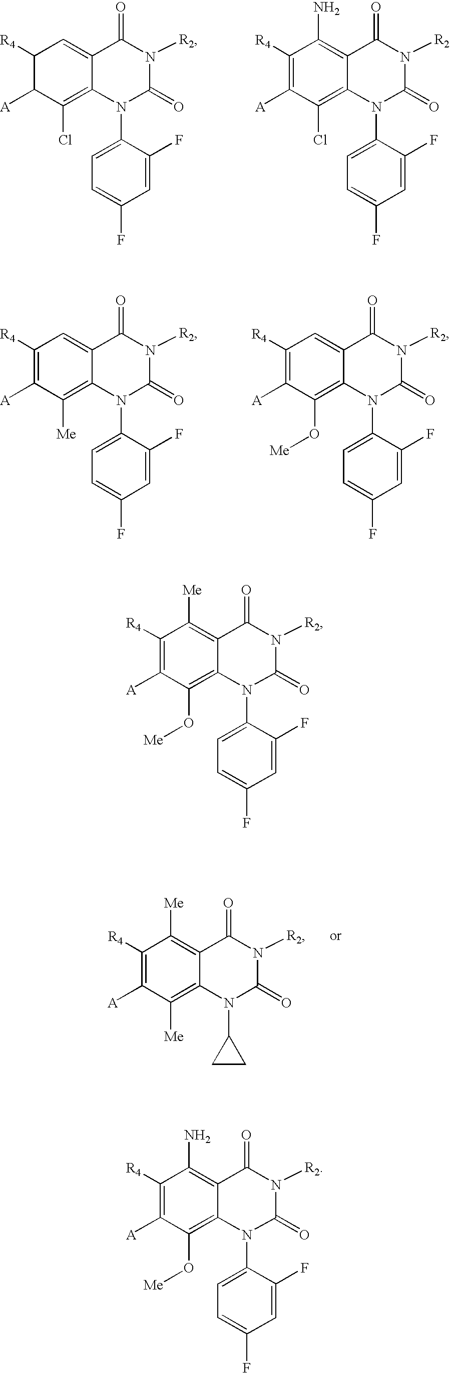

5. The compound of claim 1, wherein
z is 0, 1, 2, when q is 2 or 3 or z is 1 or 2 when q is 0, 1, 2, or 3;
Rₐ and R_{b} are each independently H, methyl, ethyl, fluoro, fluoromethyl, trifluoromethyl, fluorethyl, methoxy, MeO—N, or taken together with the carbons to which they are attached form a cyclopropyl ring;
R′, R″, R′″, and R″″ each independently H, fluoro, methyl, ethyl, fluoromethyl, fluoroethyl, phenyl, benzyl, or methoxy;
R_{c} and R_{d} each independently are H, methyl, or ethyl;
Rₑ and R_{f} each independently are H, methyl, or ethyl;
R_{g} and Rₕ each independently are H, methyl, ethyl, fluoromethyl, difluoromethyl, trifluoroethyl, cyclopropyl, phenyl, isoxazolyl, carboxymethyl, carboxyethyl, or 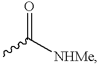
or taken together with the carbons to which they are attached form Δ; and
Rⱼ and Rₖ each independently are H, methyl, ethyl, fluoromethyl, difluoromethyl, trifluoroethyl, cyclopropyl, phenyl, isoxazolyl, carboxymethyl, carboxyethyl, or 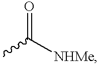
or taken together with the carbons to which they are attached form Δ.

6. The compound of claim 1, wherein A is 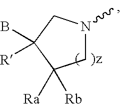
and z is 0, 1, or 2, selected from the group consisting of: 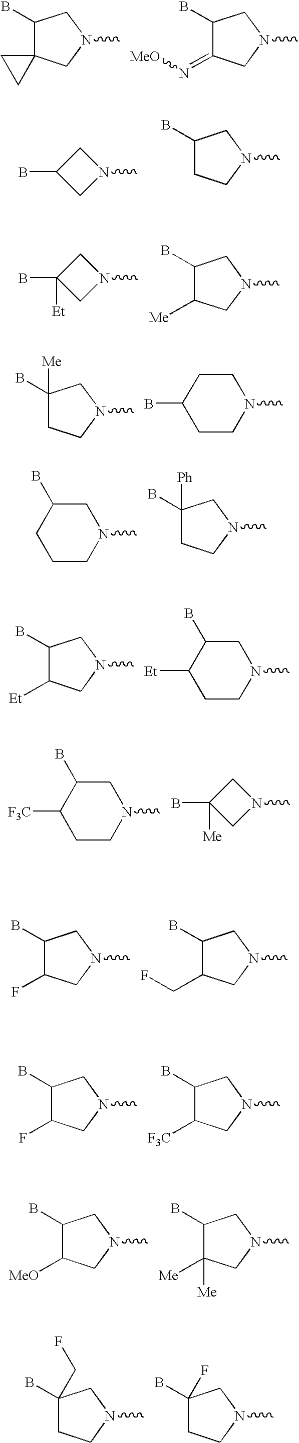 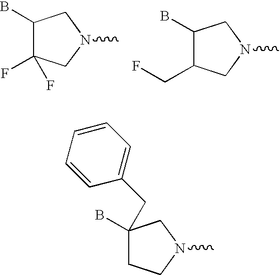
wherein [symbol] indicates the point of attachment, or A is 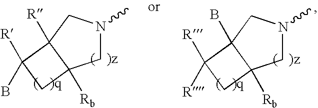
wherein z is 0, 1, or 2 and q is 0, 1, 2, or 3, selected from the group consisting of: 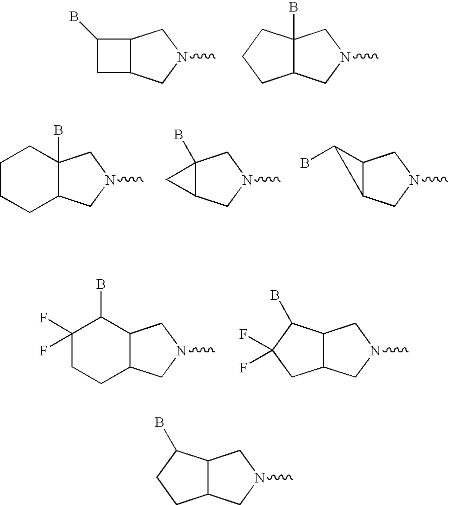
wherein [symbol] indicates the point of attachment.

7. The compound of claim 6, wherein B is 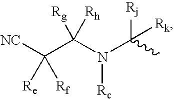
or 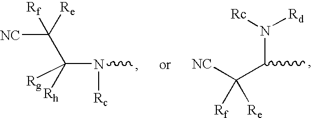
and is selected from the group consisting of: 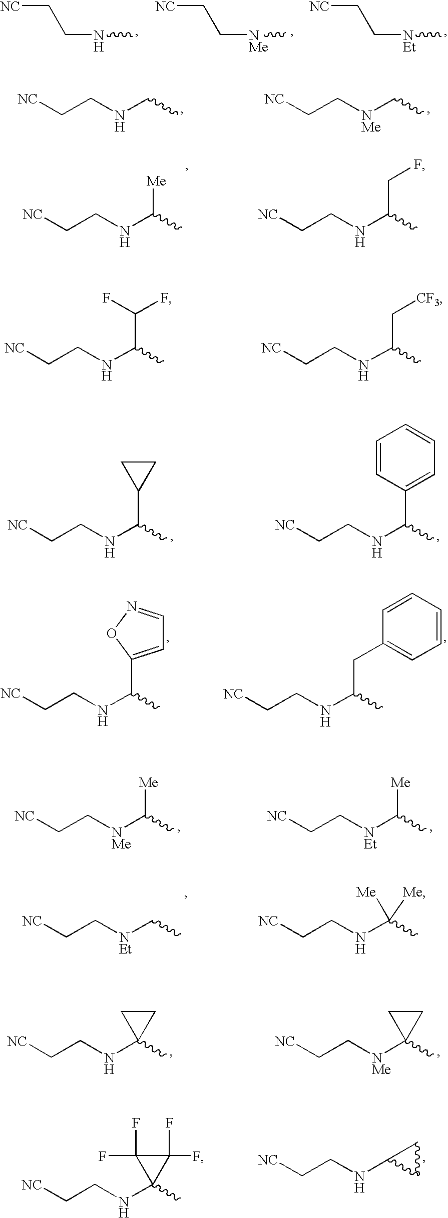 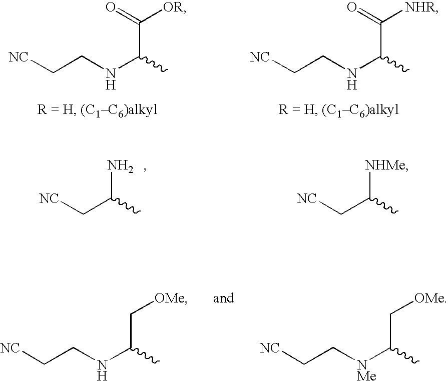
wherein [symbol] indicates the point of attachment.

8. The compound of claim 6, wherein 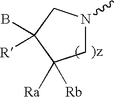
is selected from the group consisting of: 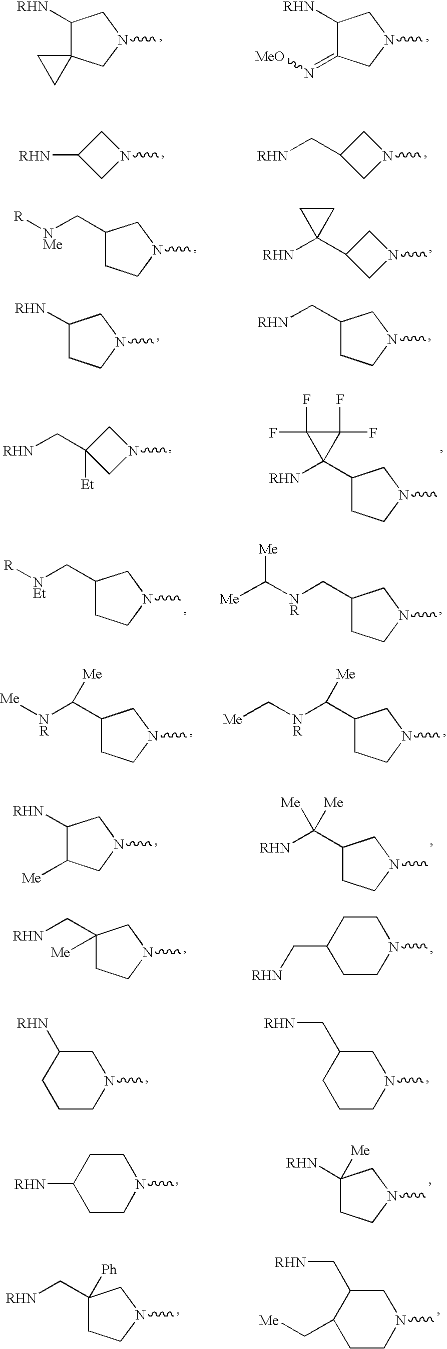 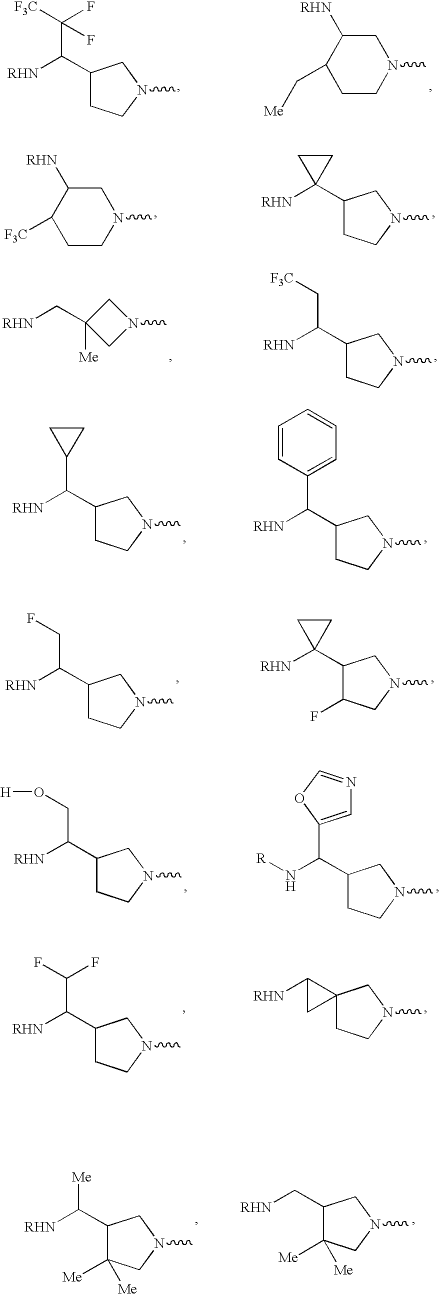 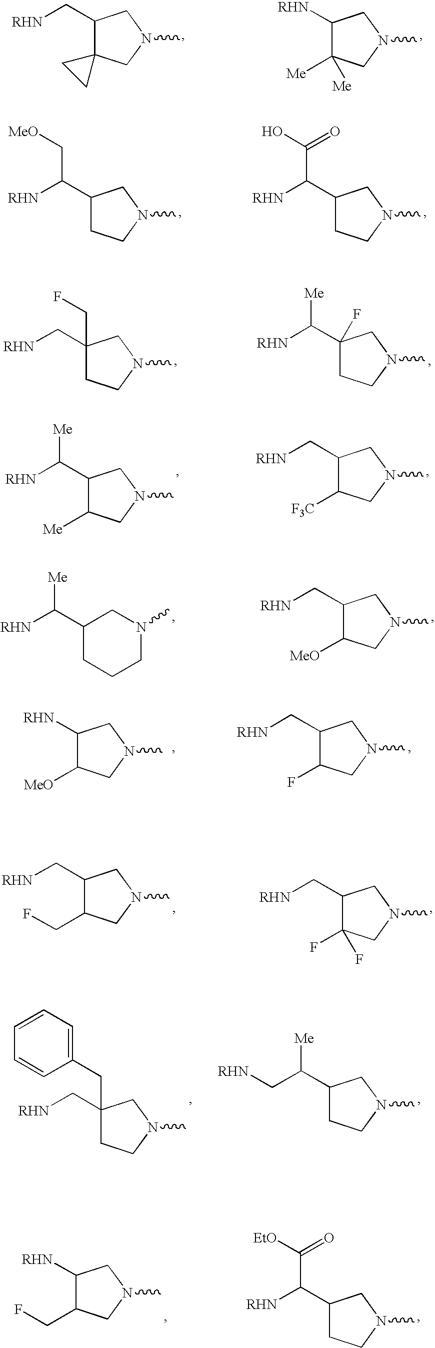 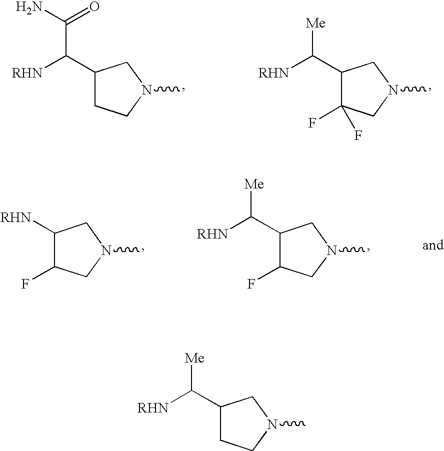
wherein R is CH₂CH₂CN and wherein [symbol] indicates the point of attachment.

9. The compound of claim 6, wherein 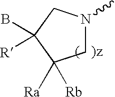
is selected from the group consisting of: 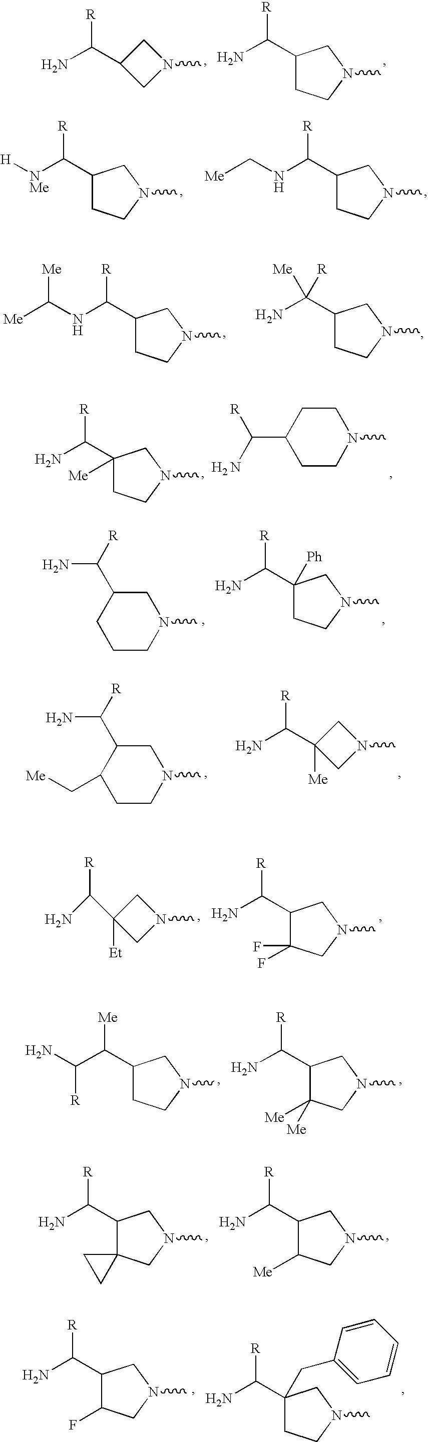 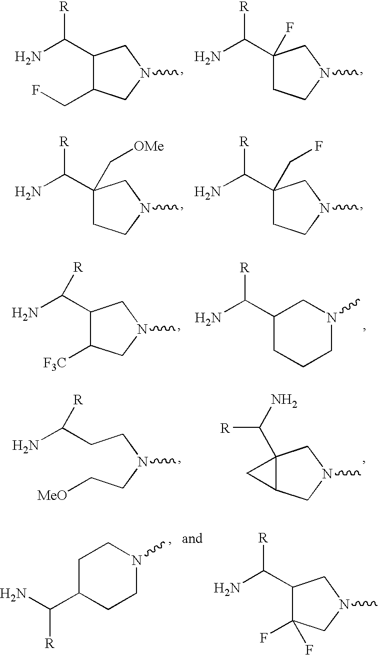
wherein R is CH₂CN, and wherein [symbol] indicates the point of attachment.

10. The compound of claim 6, wherein 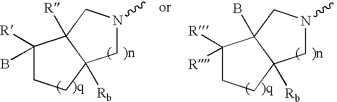
is selected from the group consisting of: 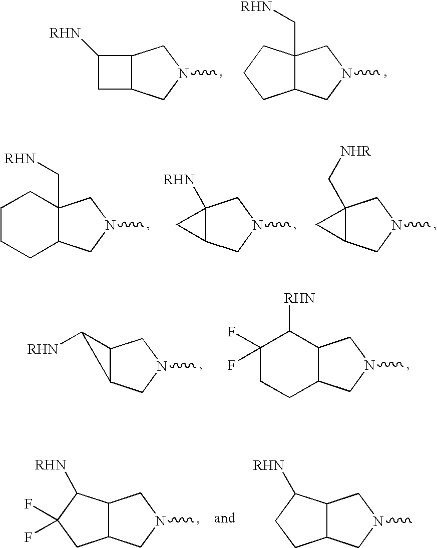
wherein R is CH₂CH₂CN, and wherein [symbol] indicates the point of attachment.

11. The compound of claim 6, wherein 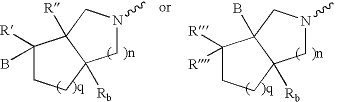
is selected from the group consisting of: 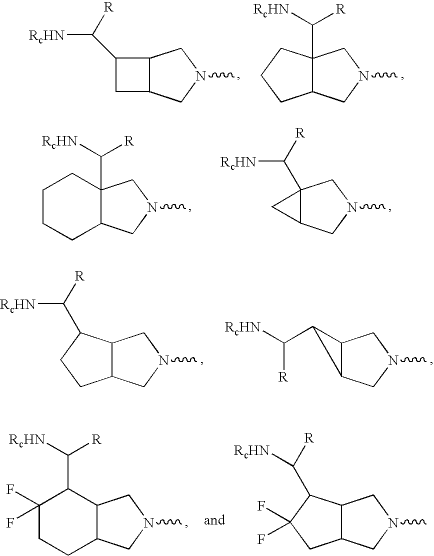
wherein R_{c} is H or (C₁-C₆)alkyl, R is CH₂CN, and wherein [symbol] indicates the point of attachment.

12. The compound of claim 1, which is a compound of formulas II, III, IV, V, or VI:

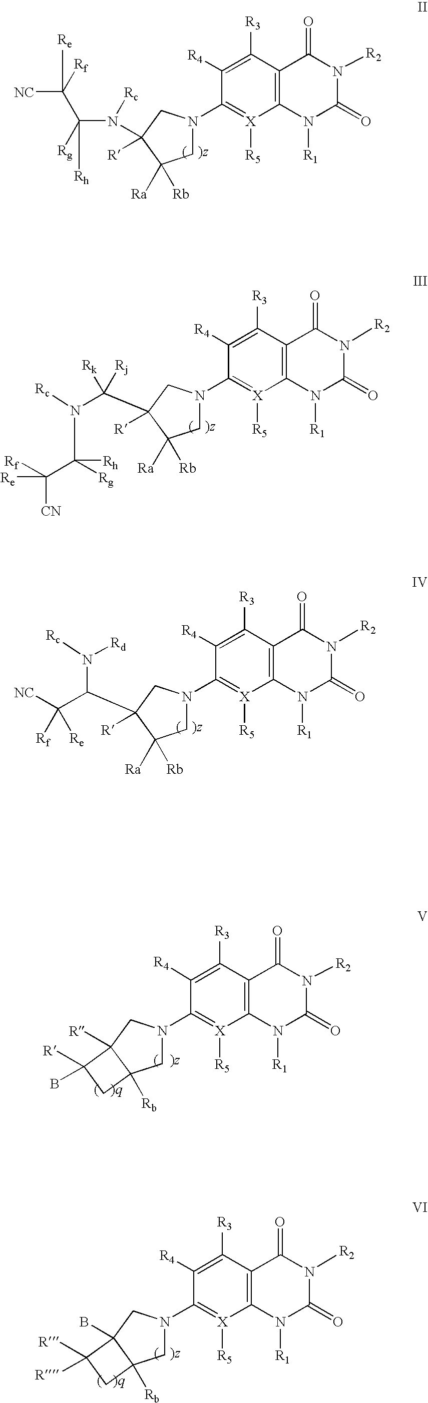

13. The compound, or a pharmaceutically acceptable salt thereof, of claim 1 selected from the group consisting of:
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-amino}-propionitrile;
3-{[1-(1-Cyclopropyl-6-fluoro-8-methoxy-2,4-dioxo-1,2,3,4-tetrahydro-quinazolin-7-yl)-pyrrolidin-3-ylmethyl]-amino}-propionitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-amino}-propionitrile;
3-{1-[1-Cyclopropyl-6-fluoro-8-methyl-2,4-dioxo-1,2,3,4-tetrahydro-quinazolin-7-yl)-pyrrolidin-3-ylmethyl]-amino}-propionitrile;
3-{[1-(3-Amino-1-cyclopropyl-6-fluoro-8-methoxy-2,4-dioxo-1,2,3,4-tetrahydro-quinazolin-7-yl)-pyrrolidin-3-yl]-ethyl]-methyl-amino]-propionitrile;
3-{[1-(1-Cyclopropyl-6-fluoro-8-methoxy-2,4-dioxo-1,2,3,4-tetrahydro-quinazolin-7-yl)-pyrrolidin-3-ylmethyl]-methyl-amino}-propionitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-propionitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-propionitrile;
3-{1-[1-(3-Amino-1-cyclopropyl-6-fluoro-8-methoxy-2,4-dioxo-1,2,3,4-tetrahydro-quinazolin-7-yl)-pyrrolidin-3-yl]-ethyl amino}-propionitrile;
3-{1-[1(1-Cyclopropyl-6-fluoro-8-methoxy-2,4-dioxo-1,2,3,4-tetrahydro-quinazolin-7-yl)-pyrrolidin-3-yl]-ethylamino}-propionitrile;
3-{1-[1-(1-Cyclopropyl-6-fluoro-8-methyl-2,4-dioxo-1,2,3,4-tetrahydro-quinazolin-7-yl)-pyrrolidin-3-yl]-ethylamino}-propionitrile;
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-ethyl}-methyl-amino)-propionitrile;
3-({1-[1-(1-Cyclopropyl-6-fluoro-8-methoxy-2,4-dioxo-1,2,3,4-tetrahydro-quinazolin-7-yl)-pyrrolidin-3-yl]-ethyl}-methyl-amino)-propionitrile;
3-({1-[1-(3-Amino-1-cyclopropyl-6-fluoro-8-methyl-2,4-dioxo-1,2,3,4-tetrahydro-quinazolin-7-yl)-pyrrolidin-3-yl]-ethyl}-methyl-amino)-propionitrile;
3-({1-[1-(1-Cyclopropyl-6-fluoro-8-methyl-2,4-dioxo-1,2,3,4-tetrahydro-quinazolin-7-yl)-pyrrolidin-3-yl]-ethyl}-methyl-amino)-propionitrile;
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-amino}-propionitrile;
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-amino}-propionitrile;
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-amino}-propionitrile;
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-amino}-propionitrile;
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-methyl-amino}-propionitrile;
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-methyl-amino}-propionitrile;
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-methyl-amino}-propionitrile;
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-methyl-amino}-propionitrile;
3-{1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropylamino}-propionitrile;
3-≡1-[1-(1-Cyclopropyl-6-fluoro-8-methoxy-2,4-dioxo-1,2,3,4-tetrahydro-quinazolin-7-yl)-pyrrolidin-3-yl]-cyclopropylamino}-propionitrile;
3-{1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropylamino}-propionitrile;
3-{1-[1-(1-Cyclopropyl-6-fluoro-8-methyl-2,4-dioxo-1,2,3,4-tetrahydro-quinazolin-7-yl)-pyrrolidin-3-yl]-cyclopropylamino}-propionitrile;
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropyl}-methyl-amino)-propionitrile;
3-({1-[1-(1-Cyclopropyl-6-fluoro-8-methoxy-2,4-dioxo-1,2,3,4-tetrahydro-quinazolin-7-yl)-pyrrolidin-3-yl]-cyclopropyl}-methyl-amino)-propionitrile;
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-methyl-amino)-propionitrile;
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-methyl-amino)-propionitrile;
N-[2-[1-(3-Amino-1-cyclopropyl-6-fluoro-8-methoxy-2,4-dioxo-1,2,3,4-tetrahydro-quinazolin-7-yl)-pyrrolidin-3-yl]-2-(2-cyano-ethylamino)-ethyl]-acetamide;
N-[2-[1-(1-Cyclopropyl-6-fluoro-8-methoxy-2,4-dioxo-1,2,3,4-tetrahydro-quinazolin-7-yl)-pyrrolidin-3-yl]-2-(2-cyano-ethylamino)-ethyl]-acetamide;
N-[2-[1-(3-Amino-1-cyclopropyl-6-fluoro-8-methyl-2,4-dioxo-1,2,3,4-tetrahydro-quinazolin-7-yl)-pyrrolidin-3-yl]-2-(2-cyano-ethylamino)-ethyl]-acetamide;
N-[2-[1-(1-Cyclopropyl-6-fluoro-8-methyl-2,4-dioxo-1,2,3,4-tetrahydro-quinazolin-7-yl)-pyrrolidin-3-yl]-2-(2-cyano-ethylamino)-ethyl]-acetamide;
N-{2-[1-(3-Amino-1-cyclopropyl-6-fluoro-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(1-Cyclopropyl-6-fluoro-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(3-Amino-1-cyclopropyl-6-fluoro-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(1-Cyclopropyl-6-fluoro-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide;
3-{[1-(3-Amino-1-cyclopropyl-8-methoxy-2,4-dioxo-1,2,3,4-tetrahydro-quinazolin-7-yl)-pyrrolidin-3-ylmethyl]-amino}-propionitrile;
3-{[1-(1-Cyclopropyl-8-methoxy-2,4-dioxo-1,2,3,4-tetrahydro-quinazolin-7-yl)-pyrrolidin-3-ylmethyl]-amino}-propionitrile;
3-{[1-(3-Amino-1-cyclopropyl-8-methyl-2,4-dioxo-1,2,3,4-tetrahydro-quinazolin-7-yl)-pyrrolidin-3-ylmethyl]-amino}-propionitrile.
3-{[1-(1-Cyclopropyl-8-methyl-2,4-dioxo-1,2,3,4-tetrahydro-quinazolin-7-yl)-pyrrolidin-3-ylmethyl]-amino}-propionitrile;
3-{[1-(3-Amino-1-cyclopropyl-8-methoxy-2,4-dioxo-1,2,3,4-tetrahydro-quinazolin-7-yl)-pyrrolidin-3-ylmethyl]-methyl-amino}-propionitrile; and
3-{[1-(1-Cyclopropyl-8-methoxy-2,4-dioxo-1,2,3,4-tetrahydro-quinazolin-7-yl)-pyrrolidin-3-ylmethyl]-methyl-amino}-propionitrile.

14. The compound, or a pharmaceutically acceptable salt thereof, of claim 1 selected from the group consisting of:
3-{[1-(3-Amino-1-cyclopropyl-8-methyl-2,4-dioxo-1,2,3,4-tetrahydro-quinazolin-7-yl)-pyrrolidin-3-ylmethyl]-methyl-amino-}-propionitrile;
3-{[1-(1-Cyclopropyl-8-methyl-2,4-dioxo-1,2,3,4-tetrahydro-quinazolin-7-yl)-pyrrolidin-3-ylmethyl]-methyl-amino}-propionitrile;
3-{1-[1-(3-Amino-1-cyclopropyl-8-methoxy-2,4-dioxo-1,2,3,4-tetrahydro-quinazolin-7-yl)-pyrrolidin-3-yl]-ethylamino}-propionitrile;
3-{1-[1-(1-Cyclopropyl-8-methoxy-2,4-dioxo-1,2,3,4-tetrahydro-quinazolin-7-yl)-pyrrolidin-3-yl]-ethylamino}-propionitrile;
3-{1-[1-(3-Amino-1-cyclopropyl-8-methyl-2,4-dioxo-1,2,3,4-tetrahydro-quinazolin-7-yl)-pyrrolidin-3-yl]-ethylamino}-propionitrile;
3-{1-[1-(1-Cyclopropyl-8-methyl-2,4-dioxo-1,2,3,4-tetrahydro-quinazolin-7-yl)-pyrrolidin-3-yl]-ethylamino}-propionitrile;
3-({1-[1-(3-Amino-1-cyclopropyl-8-methoxy-2,4-dioxo-1,2,3,4-tetrahydro-quinazolin-7-yl)-pyrrolidin-3-yl]-ethyl}-methyl-amino)-propionitrile;
3-({1-[1-(1-Cyclopropyl-8-methoxy-2,4-dioxo-1,2,3,4-tetrahydro-quinazolin-7-yl)-pyrrolidin-3-yl]-ethyl}-methyl-amino)-propionitrile;
3-({1-[1-(3-Amino-1-cyclopropyl-8-methyl-2,4-dioxo-1,2,3,4-tetrahydro-quinazolin-7-yl)-pyrrolidin-3-yl]-ethyl}-ethyl-amino)-propionitrile;
3-({1-[1-(1-Cyclopropyl-8-methyl-2,4-dioxo-1,2,3,4-tetrahydro-quinazolin-7-yl)-pyrrolidin-3-yl]-ethyl}-ethyl-amino)-propionitrile;
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-amino}-propionitrile;
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-amino}-propionitrile;
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-amino}-propionitrile;
3-{[1-(1-Cyclopropyl-8-methyl-2,4-dioxo-1,2,3,4-tetrahydro-quinazolin-7-yl)-3-methyl-pyrrolidin-3-ylmethyl]-amino}-propionitrile;
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-methyl-amino}-propionitrile;
3-{[1-(1-Cyclopropyl-8-methoxy-2,4-dioxo-1,2,3,4-tetrahydro-quinazolin-7-yl)-3-methyl-pyrrolidin-3-ylmethyl]-methyl-amino}-propionitrile;
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-methyl-amino}-propionitrile;
3-{[1-(1-Cyclopropyl-8-methyl-2,4-dioxo-1,2,3,4-tetrahydro-quinazolin-7-yl)-3-methyl-pyrrolidin-3-ylmethyl]-methyl-amino}-propionitrile;
3-{1-[1-(3-Amino-1-cyclopropyl-8-methoxy-2,4-dioxo-1,2,3,4-tetrahydro-quinazolin-7-yl)-pyrrolidin-3-yl]-cyclopropylamino}-propionitrile;
3-{1-[1-(1-Cyclopropyl-8-methoxy-2,4-dioxo-1,2,3,4-tetrahydro-quinazolin-7-yl)-pyrrolidin-3-yl]-cyclopropylamino}-propionitrile;
3-{1-[1-(3-Amino-1-cyclopropyl-8-methyl-2,4-dioxo-1,2,3,4-tetrahydro-quinazolin-7-yl)-pyrrolidin-3-yl]-cyclopropylamino}-propionitrile;
3-{1-[1-(1-Cyclopropyl-8-methyl-2,4-dioxo-1,2,3,4-tetrahydro-quinazolin-7-yl)-pyrrolidin-3-yl]-cyclopropylamino}-propionitrile;
3-({1-[1-(3-Amino-1-cyclopropyl-8-methoxy-2,4-dioxo-1,2,3,4-tetrahydro-quinazolin-7-yl)-pyrrolidin-3-yl]-cyclopropyl}-methyl-amino)-propionitrile;
3-({1-[1-(1-cyclopropyl-8-methoxy-2,4-dioxo-1,2,3,4-tetrahydro-quinazolin-7-yl)-pyrrolidin-3-yl]-cyclopropyl}-methyl-amino)-propionitrile;
3-({1-[1-(3-Amino-1-cyclopropyl-8-methyl-2,4-dioxo-1,2,3,4-tetrahydro-quinazolin-7-yl)-pyrrolidin-3-yl]-cyclopropyl}-methyl-amino)-propionitrile;
3-({1-[1-(1-Cyclopropyl-8-methyl-2,4-dioxo-1,2,3,4-tetrahydro-quinazolin-7-yl)-pyrrolidin-3-yl]-cyclopropyl}-methyl-amino)-propionitrile;
N-[2-[1-(3-Amino-1-cyclopropyl-8-methoxy-2,4-dioxo-1,2,3,4-tetrahydro-quinazolin-7-yl)-pyrrolidin-3-yl]-2-(2-cyano-ethylamino)-ethyl]-acetamide;
N-[2-[1-(1-Cyclopropyl-8-methoxy-2,4-dioxo-1,2,3,4-tetrahydro-quinazolin-7-yl)-pyrrolidin-3-yl]-2-(2-cyano-ethylamino)-ethyl]-acetamide;
N-[2-[1-(3-Amino-1-cyclopropyl-8-methyl-2,4-dioxo-1,2,3,4-tetrahydro-quinazolin-7-yl)-pyrrolidin-3-yl]-2-(2-cyano-ethylamino)-ethyl]-acetamide;
N-[2-[1-(1-Cyclopropyl-8-methyl-2,4-dioxo-1,2,3,4-tetrahydro-quinazolin-7-yl)-pyrrolidin-3-yl]-2-(2-cyano-ethylamino)-ethyl]-acetamide;
N-{2-[1-(3-Amino-1-cyclopropyl-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(1-Cyclopropyl-8-methoxy-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(3-Amino-1-cyclopropyl-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide;
N-{2-[1-(1-Cyclopropyl-8-methyl-2,4-dioxo-1,2,3,4-tetrahydro-quinazolin-7-yl)-pyrrolidin-3-yl]-2-[(2-cyano-ethyl)-methyl-amino]-ethyl}-acetamide;
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-amino}-butyronitrile;
3-{[1-(1-Cyclopropyl-6-fluoro-8-methoxy-2,4-dioxo-1,2,3,4-tetrahydro-quinazolin-7-yl)-pyrrolidin-3-ylmethyl]-amino}-butyronitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-amino}-butyronitrile;
3-{[1-(1-Cyclopropyl-6-fluoro-8-methyl-2,4-dioxo-1,2,3,4-tetrahydro-quinazolin-7-yl)-pyrrolidin-3-ylmethyl]-amino}-butyronitrile;
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-methyl-amino}-butyronitrile;
3-{[1-(1-Cyclopropyl-6-fluoro-8-methoxy-2,4-dioxo-1,2,3,4-tetrahydro-quinazolin-7-yl)-pyrrolidin-3-ylmethyl]-methyl-amino}-butyronitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-methyl-amino}-butyronitrile;
3-{[1-(1-Cyclopropyl-6-fluoro-8-methyl-2,4-dioxo-1,2,3,4-tetrahydro-quinazolin-7-yl)-pyrrolidin-3-ylmethyl]-methyl-amino}-butyronitrile;
3-[5-(3-Amino-1-cyclopropyl-6-fluoro-8-methoxy-2,4-dioxo-1,2,3,4-tetrahydro-quinazolin-7-yl)-5-aza-spiro[2.4]hept-1-ylamino]-propionitrile;
3-[5-(1-Cyclopropyl-6-fluoro-8-methoxy-2,4-dioxo-1,2,3,4-tetrahydro-quinazolin-7-yl)-5-aza-spiro[2.4]hept-1-ylamino]-propionitrile; and
3-[5-(3-Amino-1-cyclopropyl-6-fluoro-8-methyl-2,4-dioxo-1,2,3,4-tetrahydro-quinazolin-7-yl)-5-aza-spiro[2.4]hept-1-ylamino]-propionitrile.

15. The compound, or a pharmaceutically acceptable salt thereof of claim 1 selected from the group consisting of:
3-[5-(1-Cyclopropyl-6-fluoro-8-methyl-2,4-dioxo-1,2,3,4-tetrahydro-quinazolin-7-yl)-5-aza-spiro[2.4]hept-1-ylamino]-propionitrile;
3-{1-[1-(3-Amino-1-cyclopropyl-6-fluoro-8-methoxy-2,4-dioxo-1,2,3,4-tetrahydro-quinazolin-7-yl)-azetidin-3-yl]-propylamino}-propionitrile;
3-{1-[1-(1-Cyclopropyl-6-fluoro-8-methoxy-2,4-dioxo-1,2,3,4-tetrahydro-quinazolin-7-yl)-azetidin-3-yl]-propylamino}-propionitrile;
3-{1-[1-(3-Amino-1-cyclopropyl-6-fluoro-8-methyl-2,4-dioxo-1,2,3,4-tetrahydro-quinazolin-7-yl)-azetidin-3-yl]-propylamino}-propionitrile;
3-{1-[1-(1-Cyclopropyl-6-fluoro-8-methyl-2,4-dioxo-1,2,3,4-tetrahydro-quinazolin-7-yl)-azetidin-3-yl]-propylamino}-propionitrile;
3-({1-[1-(3-Amino-1-cyclopropyl-6-fluoro-8-methoxy-2,4-dioxo-1,2,3,4-tetrahydro-quinazolin-7-yl)-azetidin-3-yl]-propyl}-methyl-amino)-propionitrile.
3-({1-[1-(1-Cyclopropyl-6-fluoro-8-methoxy-2,4-dioxo-1,2,3,4-tetrahydro-quinazolin-7-yl)-azetidin-3-yl]-propyl}-methyl-amino)-propionitrile;
3-({1-[1-(3-Amino-1-cyclopropyl-6-fluoro-8-methyl-2,4-dioxo-1,2,3,4-tetrahydro-quinazolin-7-yl)-azetidin-3-yl]-propyl}-methyl-amino)-propionitrile;
3-({1-[1-(1-Cyclopropyl-6-fluoro-8-methyl-2,4-dioxo-1,2,3,4-tetrahydro-quinazolin-7-yl)-azetidin-3-yl]-propyl}-methyl-amino)-propionitrile;
3-{1-[1-(3-Amino-1-cyclopropyl-6-fluoro-8-methoxy-2,4-dioxo-1,2,3,4-tetrahydro-quinazolin-7-yl)-azetidin-3-yl]-cylopropylamino}-propionitrile;
3-{1-[1-(1-Cyclopropyl-6-fluoro-8-methoxy-2,4-dioxo-1,2,3,4-tetrahydro-quinazolin-7-yl)-azetidin-3-yl]-cyclopropylamino}-propionitrile;
3-{1-[1-(3-Amino-1-cyclopropyl-6-fluoro-8-methyl-2,4-dioxo-1,2,3,4-tetrahydro-quinazolin-7-yl)-azetidin-3-yl]-cyclopropylamino}-propionitrile;
3-{1-[1-(1-Cyclopropyl-6-fluoro-8-methyl-2,4-dioxo-1,2,3,4-tetrahydro-quinazolin-7-yl)-azetidin-3-yl]-cyclopropylamino}-propionitrile;
3-({1-[1-(3-Amino-1-cyclopropyl-6-fluoro-8-methoxy-2,4-dioxo-1,2,3,4-tetrahydro-quinazolin-7-yl)-azetidin-3-yl]-cyclopropyl}-methyl-amino)-propionitrile;
3-({1-[1-(1-Cyclopropyl-6-fluoro-8-methoxy-2,4-dioxo-1,2,3,4-tetrahydro-quinazolin-7-yl)-azetidin-3-yl]-cyclopropyl}-methyl-amino)-propionitrile;
3-({1-[1-(3-Amino-1-cyclopropyl-6-fluoro-8-methyl-2,4-dioxo-1,2,3,4-tetrahydro-quinazolin-7-yl)-azetidin-3-yl]-cyclopropyl}-methyl-amino)-propionitrile;
3-({1-[1-(1-Cyclopropyl-6-fluoro-8-methyl-2,4-dioxo-1,2,3,4-tetrahydro-quinazolin-7-yl)-azetidin-3-yl]-cyclopropyl)-methyl-amino)-propionitrile;
3-({1-[1-(3-Amino-1-cyclopropyl-6-fluoro-8-methoxy-2,4-dioxo-1,2,3,4-tetrahydro-quinazolin-7-yl)-azetidin-3-yl]-cyclopropyl}-ethyl-amino)-propionitrile;
3-({1-[1-(1-Cyclopropyl-6-fluoro-8-methoxy-2,4-dioxo-1,2,3,4-tetrahydro-quinazolin-7-yl)-azetidin-3-yl]-cyclopropyl}-ethyl-amino)-propionitrile;
3-({1-[1-(3-Amino-1-cyclopropyl-6-fluoro-8-methyl-2,4-dioxo-1,2,3,4-tetrahydro-quinazolin-7-yl)-azetidin-3-yl]-cyclopropyl}ethyl-amino)-propionitrile;
3-({1-[1-(1-Cyclopropyl-6-fluoro-8-methyl-2,4-dioxo-1,2,3,4-tetrahydro-quinazolin-7-yl)-azetidin-3-yl]-cyclopropyl}-ethyl-amino)-propionitrile;
3-{[1-(3-Amino-1-cyclopropyl-6-fluoro-8-methoxy-2,4-dioxo-1,2,3,4-tetrahydro-quinazolin-7-yl)-pyrrolidin-3-ylmethyl]-ethyl-amino}-propionitrile;
3-{[1-(1-Cyclopropyl-6-fluoro-8-methoxy-2,4-dioxo-1,2,3,4-tetrahydro-quinazolin-7-yl)-pyrrolidin-3-ylmethyl]-ethyl-amino}-propionitrile;
3-{[1-(3-Amino-1-cyclopropyl-6-fluoro-8-methyl-2,4-dioxo-1,2,3,4-tetrahydro-quinazolin-7-yl)-pyrrolidin-3-ylmethyl]-ethyl-amino}-propionitrile;
3-{[1-(1-Cyclopropyl-6-fluoro-8-methyl-2,4-dioxo-1,2,3,4-tetrahydro-quinazolin-7-yl)-pyrrolidin-3-ylmethyl]-ethyl-amino}-propionitrile;
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-ethyl}-ethyl-amino)-propionitrile;
3-({1-[1-(1-Cyclopropyl-6-fluoro-8-methoxy-2,4-dioxo-1,2,3,4-tetrahydro-quinazolin-7-yl)-pyrrolidin-3-yl]-ethyl}-ethyl-amino)-propionitrile;
3-({1-[1-(3-Amino-1-cyclopropyl-6-fluoro-8-methyl-2,4-dioxo-1,2,3,4-tetrahydro-quinazolin-7-yl)-pyrrolidin-3-yl]-ethyl}-ethyl-amino)-propionitrile;
3-({1-[1-(1-Cyclopropyl-6-fluoro-8-methyl-2,4-dioxo-1,2,3,4-tetrahydro-quinazolin-7-yl)-pyrrolidin-3-yl]-ethyl}-ethyl-amino)-propionitrile;
3-{[1-(3-Amino-1-cyclopropyl-6-fluoro-8-methoxy-2,4-dioxo-1,2,3,4-tetrahydro-quinazolin-7-yl)-3-methyl-pyrrolidin-3-ylmethyl]-ethyl-amino}-propionitrile;
3-{[1-(1-Cyclopropyl-6-fluoro-8-methoxy-2,4-dioxo-1,2,3,4-tetrahydro-quinazolin-7-yl)-3-methyl-pyrrolidin-3-ylmethyl]-ethyl-amino}-propionitrile;
3-{[1-(3-Amino-1-cyclopropyl-6-fluoro-8-methyl-2,4-dioxo-1,2,3,4-tetrahydro-quinazolin-7-yl)-3-methyl-pyrrolidin-3-ylmethyl]-ethyl-amino}-propionitrile;
3-{[1-(1-Cyclopropyl-6-fluoro-8-methyl-2,4-dioxo-1,2,3,4-tetrahydro-quinazolin-7-yl)-3-methyl-pyrrolidin-3-ylmethyl]-ethyl-amino}-propionitrile;
3-({1-[1-(3-Amino-1-cyclopropyl-6-fluoro-8-methoxy-2,4-dioxo-1,2,3,4-tetrahydro-quinazolin-7-yl)-pyrrolidin-3-yl]-cyclopropyl}-ethyl-amino)-propionitrile;
3-({1-[1-(1-Cyclopropyl-6-fluoro-8-methoxy-2,4-dioxo-1,2,3,4-tetrahydro-quinazolin-7-yl)-pyrrolidin-3-yl]-cyclopropyl}-ethyl-amino)-propionitrile;
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile;
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile;
N-{2-[1-(3-Amino-1-cyclopropyl-6-fluoro-5-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide;
N-{2-[1-(1-Cyclopropyl-6-fluoro-8-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide;
N-{2-[1-(3-Amino-1-cyclopropyl-6-fluoro-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide;
N-{2-[1-(1-Cyclopropyl-6-fluoro-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide;
3-{[1-(3-Amino-1-cyclopropyl-8-methoxy-2,4-dioxo-1,2,3,4-tetrahydro-quinazolin-7-yl)-pyrrolidin-3-ylmethyl]-ethyl-amino}-propionitrile,
3-{[1-(1-Cyclopropyl-8-methoxy-2,4-dioxo-1,2,3,4-tetrahydro-quinazolin-7-yl)-pyrrolidin-3-ylmethyl]-ethyl-amino}-propionitrile;
3-({1-[1-(3-Amino-1-cyclopropyl-8-methoxy-2,4-dioxo-1,2,3,4-tetrahydro-quinazolin-7-yl)-pyrrolidin-3-yl]-ethyl}-ethyl-amino)-propionitrile; and
3-({1-[1-(1-Cyclopropyl-8-methoxy-2,4-dioxo-1,2,3,4-tetrahydro-quinazolin-7-yl)-pyrrolidin-3-yl]-ethyl}-ethyl-amino)-propionitrile.

16. The compound, or a pharmaceutically acceptable salt thereof, of claim 1 selected from the group consisting of:
3-({1-[1-(3-Amino-1-cyclopropyl-8-methyl-2,4-dioxo-1,2,3,4-tetrahydro-quinazolin-7-yl)-pyrrolidin-3-yl]-ethyl}-methyl-amino)-propionitrile;
3-({1-[1-(1-Cyclopropyl-8-methyl-2,4-dioxo-1,2,3,4-tetrahydro-quinazolin-7-yl)-pyrrolidin-3-yl]-ethyl}-methyl-amino)-propionitrile;
3-{[1-(3-Amino-1-cyclopropyl-8-methoxy-2,4-dioxo-1,2,3,4-tetrahydro-quinazolin-7-yl)-3-methyl-pyrrolidin-3-ylmethyl]-ethyl-amino}-propionitrile;
3-{[1-(1-Cyclopropyl-8-methoxy-2,4-dioxo-1,2,3,4-tetrahydro-quinazolin-7-yl)-3-methyl-pyrrolidin-3-ylmethyl]-ethyl-amino}-propionitrile;
3-{[1-(3-Amino-1-cyclopropyl-8-methyl-2,4-dioxo-1,2,3,4-tetrahydro-quinazolin-7-yl)-3-methyl-pyrrolidin-3-ylmethyl]-ethyl-amino}-propionitrile;
3-{[1-(1-Cyclopropyl-8-methyl-2,4-dioxo-1,2,3,4-tetrahydro-quinazolin-7-yl)-3-methyl-pyrrolidin-3-ylmethyl]-ethyl-amino}-propionitrile;
3-({1-[1-(3-Amino-1-cyclopropyl-8-methoxy-2,4-dioxo-1,2,3,4-tetrahydro-quinazolin-7-yl)-pyrrolidin-3-yl]-cyclopropyl}-ethyl-amino)-propionitrile;
3-({1-[1-(1-Cyclopropyl-8-methoxy-2,4-dioxo-1,2,3,4-tetrahydro-quinazolin-7-yl)-pyrrolidin-3-yl]-cyclopropyl}-ethyl-amino)-propionitrile;
3-({1-[1-(3-Amino-1-cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile;
3-({1-[1-(1-Cyclopropyl-6-fluoro-8-methyl-2,4-dioxo-1,2,3,4-tetrahydro-quinazolin-7-yl)-pyrrolidin-3-yl]-cyclopropyl}-ethyl-amino)-propionitrile;
N-{2-[1-(3-Amino-1-cyclopropyl-8-methoxy-2,4-dioxo-1,2,3,4-tetrahydro-quinazolin-7-yl)-pyrrolidin-3-yl]-2-[(2-cyano-ethyl)-ethyl-amino]-ethyl}-acetamide;
N-{2-[(2-Cyano-ethyl)-ethyl-amino]-2-[1-(1-cyclopropyl-8-methoxy-2,4-dioxo-1,2,3,4-tetrahydro-quinazolin-7-yl)-pyrrolidin-3-yl]-ethyl}-acetamide;
N-{2-[1-(3-Amino-1-cyclopropyl-8-methyl-2,4-dioxo-1,2,3,4-tetrahydro-quinazolin-7-yl)-pyrrolidin-3-yl]-2-[(2-cyano-ethyl)-ethyl-amino]-ethyl}-acetamide;
N-{2-[(2-Cyano-ethyl)-ethyl-amino]-2-[1-(1-cyclopropyl-8-methyl-2,4-dioxo-1,2,3,4-tetrahydro-quinazolin-7-yl)-pyrrolidin-3-yl]-ethyl}-acetamide;
3-{1-[1-(3-Amino-1-cyclopropyl-8-methyl-2,4-dioxo-1,2,3,4-tetrahydro-quinazolin-7-yl)-azetidin-3-yl]-propylamino}-propionitrile;
3-{1-[1-(1-Cyclopropyl-8-methyl-2,4-dioxo-1,2,3,4-tetrahydro-quinazolin-7-yl)-azetidin-3-yl]-propylamino}-propionitrile;
3-{[1-(3-Amino-1-cyclopropyl-8-methyl-2,4-dioxo-1,2,3,4-tetrahydro-quinazolin-7-yl)-3-methyl-azetidin-3-ylmethyl]-amino}-propionitrile;
3-{[1-(1-Cyclopropyl-8-methyl-2,4-dioxo-1,2,3,4-tetrahydro-quinazolin-7-yl)-3-methyl-azetidin-3-ylmethyl]-amino}-propionitrile;
3-{1-[1-(3-Amino-1-cyclopropyl-8-methyl-2,4-dioxo-1,2,3,4-tetrahydro-quinazolin-7-yl)-azetidin-3-yl]-cyclopropylamino}-propionitrile;
3-{1-[1-(1-Cyclopropyl-8-methyl-2,4-dioxo-1,2,3,4-tetrahydro-quinazolin-7-yl)-azetidin-3-yl]-cyclopropylamino}-propionitrile;
3-{[1-(3-Amino-1-cyclopropyl-8-methyl-2,4-dioxo-1,2,3,4-tetrahydro-quinazolin-7-yl)-3-ethyl-azetidin-3-ylmethyl]-amino}-propionitrile;
3-[1-(1-Cyclopropyl-8-methyl-2,4-dioxo-1,2,3,4-tetrahydro-quinazolin-7-yl)-3-ethyl-azetidin-3-ylmethyl]-amino}-propionitrile=;
3-Amino-3-[1-(5-amino-8-fluoro-3-methyl-4,6-dioxo-2,3,5,6-tetrahydro-4H-1-oxa-3a,5-diaza-phenalen-9-yl)-pyrolidin-3-yl]-propionitrile;
3-Amino-3-[1-(8-fluoro-3-methyl-4,6-dioxo-2,3,5,6-tetrahydro-4H-1-oxa-3a,5-diaza-phenalen-9-yl)-pyrolidin-3-yl]-propionitrile;
3-Amino-3-[1-(3-amino-1-cyclopropyl-6-fluoro-2,4-dioxo-1,2,3,4-tetrahydro-pyrido[2,3-d]pyrimidin-7-yl)-pyrrolidin-3-yl]-propionitrile;
3-Amino-3-[1-(1-cyclopropyl-6-fluoro-2,4-dioxo-1,2,3,4-tetrahydro-pyrido[2,3-d]pyrimidin-7-yl)-pyrrolidin-3-yl]-propionitrile;
3-Amino-3-[1-(3-amino-1-cyclopropyl-6-fluoro-8-methoxy-2,4-dioxo-1,2,3,4-tetrahydro-quinazolin-7-yl)-pyrrolidin-3-yl]-propionitrile;
3-Amino-3-[1-(1-cyclopropyl-6-fluoro-8-methoxy-2,4-dioxo-1,2,3,4-tetrahydro-quinazolin-7-yl)-pyrrolidin-3-yl]-propionitrile;
3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-propionitrile;
3-Amino-3-[1-(1-cyclopropyl-6-fluoro-8-methyl-2,4-dioxo-1,2,3,4-tetrahydro-quinazolin-7-yl)-pyrrolidin-3-yl]-propionitrile;
3-Amino-3-[1-(3-amino-1-cyclopropyl-6-fluoro-2,4-dioxo-1,2,3,4-tetrahydro-quinazolin-7-yl)-pyrrolidin-3-yl]-propionitrile;
3-Amino-3-[1-(1-cyclopropyl-6-fluoro-2,4-dioxo-1,2,3,4-tetrahydro-quinazolin-7-yl)-pyrrolidin-3-yl]-propionitrile;
3-Amino-3-[1-(3-amino-1-cyclopropyl-8-methoxy-2,4-dioxo-1,2,3,4-tetrahydro-quinazolin-7-yl)-pyrrolidin-3-yl]-propionitrile;
3-Amino-3-[1-(1-cyclopropyl-8-methoxy-2,4-dioxo-1,2,3,4-tetrahydro-quinazolin-7-yl)-pyrrolidin-3-yl]-propionitrile;
3-Amino-3-[1-(3-amino-1-cyclopropyl-8-methyl-2,4-dioxo-1,2,3,4-tetrahydro-quinazolin-7-yl)-pyrrolidin-3-yl]-propionitrile;
3-Amino-3-[1-(1-cyclopropyl-8-methyl-2,4-dioxo-1,2,3,4-tetrahydro-quinazolin-7-yl)-pyrrolidin-3-yl]-propionitrile;
3-Amino-3-[1-(3,5-diamino-1-cyclopropyl-8-methoxy-2,4-dioxo-1,2,3,4-tetrahydro-quinazolin-7-yl)-pyrrolidin-3-yl]-propionitrile;
3-Amino-3-[1-(5-amino-1-cyclopropyl-8-methoxy-2,4-dioxo-1,2,3,4-tetrahydro-quinazolin-7-yl)-pyrrolidin-3-yl]-propionitrile;
3-Amino-3-[1-(3,5-diamino-1-cyclopropyl-8-methyl-2,4-dioxo-1,2,3,4-tetrahydro-quinazolin-7-yl)-pyrrolidin-3-yl]-propionitrile;
3-Amino-3-[1-(5-amino-1-cyclopropyl-8-methyl-2,4-dioxo-1,2,3,4-tetrahydro-quinazolin-7-yl)-pyrrolidin-3-yl]-propionitrile;
3-Amino-3-[1-(3-amino-1-cyclopropyl-6-fluoro-8-methoxy-2,4-dioxo-1,2,3,4-tetrahydro-quinazolin-7-yl)-pyrrolidin-3-yl]-2,2-dimethyl-propionitrile;
3-Amino-3-[1-(1-Cyclopropyl-6-fluoro-8-methoxy-2,4-dioxo-1,2,3,4-tetrahydro-quinazolin-7-yl)-pyrrolidin-3-yl]-2,2-dimethyl-propionitrile;
3-Amino-3-[1-(3-amino-1-cyclopropyl-6-fluoro-8-methyl-2,4-dioxo-1,2,3,4-tetrahydro-quinazolin-7-yl)-pyrrolidin-3-yl]-2,2-dimethyl-propionitrile;
3-Amino-3-[1-(1-cyclopropyl-6-fluoro-8-methyl-2,4-dioxo-1,2,3,4-tetrahydro-quinazolin-7-yl)-pyrrolidin-3-yl]-2,2-dimethyl-propionitrile; and
3-Amino-3-[1-(3-amino-1-cyclopropyl-8-methoxy-2,4-dioxo-1,2,3,4-tetrahydro-quinazolin-7-yl)-pyrrolidin-3-yl]-2,2-dimethyl-propionitrile.

17. The compound, or a pharmaceutically acceptable salt thereof, of claim 1 selected from the group consisting of:
3-Amino-3-[1-(1-cyclopropyl-8-methoxy-2,4-dioxo-1,2,3,4-tetrahydro-quinazolin-7-yl)-pyrrolidin-3-yl]-2,2-dimethyl-propionitrile;
3-Amino-3-[1-(3-amino-1-cyclopropyl-8-methyl-2,4-dioxo-1,2,3,4-tetrahydro-quinazolin-7-yl)-pyrrolidin-3-yl]-2,2-dimethyl-propionitrile;
3-Amino-3-[1-(1-cyclopropyl-8-methyl-2,4-dioxo-1,2,3,4-tetrahydro-quinazolin-7-yl)-pyrrolidin-3-yl]-2,2-dimethyl-propionitrile;
3-Amino-3-[1-(3,5-diamino-1-cyclopropyl-8-methoxy-2,4-dioxo-1,2,3,4-tetrahydro-quinazolin-7-yl)-pyrrolidin-3-yl]-2,2-dimethyl-propionitrile;
3-Amino-3-[1-(5-amino-1-cyclopropyl-8-methoxy-2,4-dioxo-1,2,3,4-tetrahydro-quinazolin-7-yl)-pyrrolidin-3-yl]-2,2-dimethyl-propionitrile;
3-Amino-3-[1-(3,5-diamino-1-cyclopropyl-8-methyl-2,4-dioxo-1,2,3,4-tetrahydro-quinazolin-7-yl)-pyrrolidin-3-yl]-2,2-dimethyl-propionitrile;
3-Amino-3-[1-(5-amino-1-cyclopropyl-8-methyl-2,4-dioxo-1,2,3,4-tetrahydro-quinazolin-7-yl)-pyrrolidin-3-yl]-2,2-dimethyl-propionitrile;
3-Amino-3-[1-(3-amino-1-cyclopropyl-6-fluoro-2,4-dioxo-1,2,3,4-tetrahydro-quinazolin-7-yl)-pyrrolidin-3-yl]-propionitrile;
3-Amino-3-[1-(1-cyclopropyl-6-fluoro-2,4-dioxo-1,2,3,4-tetrahydro-quinazolin-7-yl)-pyrrolidin-3-yl]-propionitrile;
3-[1-(3-Amino-1-cyclopropyl-6-fluoro-2,4-dioxo-1,2,3,4-tetrahydro-quinazolin-7-yl)-pyrrolidin-3-yl]-3-methylamino-propionitrile;
3-[1-(1-cyclopropyl-6-fluoro-2,4-dioxo-1,2,3,4-tetrahydro-quinazolin-7-yl)-pyrrolidin-3-yl]-3-methylamino-propionitrile;
3-[1-(5-Amino-8-fluoro-3-methyl-4,6-dioxo-2,3,5,6-tetrahydro-4H-1-oxa-3a,5-diaza-phenalen-9-yl)-pyrrolidin-3-yl]-3-methylamino-propionitrile;
3-[1-(8-Fluoro-3-methyl-4,6-dioxo-2,3,5,6-tetrahydro-4H-1-oxa-3a,5-diaza-phenalen-9-yl)-pyrrolidin-3-yl]-3-methylamino-propionitrile;
3-[1-(3-Amino-1-cyclopropyl-6-fluoro-2,4-dioxo-1,2,3,4-tetrahydro-pyrido[2,3-d]pyrimidin-7-yl)-pyrrolidin-3-yl]-3-methylamino-propionitrile;
3-[1-(1-cyclopropyl-6-fluoro-2,4-dioxo-1,2,3,4-tetrahydro-pyrido[2,3-d]pyrimidin-7-yl)-pyrrolidin-3-yl]-3-methylamino-propionitrile;
3-[1-(3-Amino-1-cyclopropyl-6-fluoro-8-methoxy-2,4-dioxo-1,2,3,4-tetrahydro-quinazolin-7-yl)-pyrrolidin-3-yl]-3-methylamino-propionitrile;
3-[1-(1-cyclopropyl-6-fluoro-8-methoxy-2,4-dioxo-1,2,3,4-tetrahydro-quinazolin-7-yl)-pyrrolidin-3-yl]-3-methylamino-propionitrile;
3-[1-(3-Amino-1-cyclopropyl-6-fluoro-8-methyl-2,4-dioxo-1,2,3,4-tetrahydro-quinazolin-7-yl)-pyrrolidin-3-yl]-3-methylamino-propionitrile;
3-[1-(1-cyclopropyl-6-fluoro-8-methyl-2,4-dioxo-1,2,3,4-tetrahydro-quinazolin-7-yl)-pyrrolidin-3-yl]-3-methylamino-propionitrile;
3-[1-(3-Amino-1-cyclopropyl-8-methoxy-2,4-dioxo-1,2,3,4-tetrahydro-quinazolin-7-yl)-pyrrolidin-3-yl]-3-methylamino-propionitrile;
3-[1-(1-cyclopropyl-8-methoxy-2,4-dioxo-1,2,3,4-tetrahydro-quinazolin-7-yl)-pyrrolidin-3-yl]-3-methylamino-propionitrile;
3-[1-(3-Amino-1-cyclopropyl-8-methyl-2,4-dioxo-1,2,3,4-tetrahydro-quinazolin-7-yl)-pyrrolidin-3-yl]-3-methylamino-propionitrile;
3-[1-(1-cyclopropyl-8-methyl-2,4-dioxo-1,2,3,4-tetrahydro-quinazolin-7-yl)-pyrrolidin-3-yl]-3-methylamino-propionitrile;
3-[1-(3,5-Diamino-1-cyclopropyl-8-methoxy-2,4-dioxo-1,2,3,4-tetrahydro-quinazolin-7-yl)-pyrrolidin-3-yl]-3-methylamino-propionitrile;
3-[1-(5-amino-1-cyclopropyl-8-methoxy-2,4-dioxo-1,2,3,4-tetrahydro-quinazolin-7-yl)-pyrolidin-3-yl]-3-methylamino-propionitrile;
3-[1-(3,5-Diamino-1-cyclopropyl-8-methyl-2,4-dioxo-1,2,3,4-tetrahydro-quinazolin-7-yl)-pyrrolidin-3-yl]-3-methylamino-propionitrile;
3-[1-(5-amino-1-cyclopropyl-8-methyl-2,4-dioxo-1,2,3,4-tetrahydro-quinazolin-7-yl)-pyrrolidin-3-yl]-3-methylamino-propionitrile;
3-[1-(3-Amino-1-cyclopropyl-6-fluoro-8-methoxy-2,4-dioxo-1,2,3,4-tetrahydro-quinazolin-7-yl)-pyrrolidin-3-yl]-2,2-dimethyl-3-methylamino-propionitrile;
3-[1-(1-Cyclopropyl-6-fluoro-8-methoxy-2,4-dioxo-1,2,3,4-tetrahydro-quinazolin-7-yl)-pyrrolidin-3-yl]-2,2-dimethyl-3-dimethylamino-propionitrile;
3-[1-(3-Amino-1-cyclopropyl-6-fluoro-8-methyl-2,4-dioxo-1,2,3,4-tetrahydro-quinazolin-7-yl)-pyrrolidin-3-yl]-2,2-dimethyl-3-methylamino-propionitrile;
3-[1-(1-Cyclopropyl-6-fluoro-8-methyl-2,4-dioxo-1,2,3,4-tetrahydro-quinazolin-7-yl)-pyrrolidin-3-yl]-2,2-dimethyl-3-methylamino-propionitrile;
3-[1-(3-Amino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile;
3-[1-(1-Cyclopropyl-8-methoxy-2,4-dioxo-1,2,3,4-tetrahydro-quinazolin-7-yl)-pyrrolidin-3-yl]-2,2-dimethyl-3-methylamino-propionitrile;
3-[1-(3-Amino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile;
3-[1-(1-Cyclopropyl-8-methyl-2,4-dioxo-1,2,3,4-tetrahydro-quinazolin-7-yl)-pyrrolidin-3-yl]-2,2-dimethyl-3-methylamino-propionitrile;
3-[1-(3,5-Diamino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile;
3-[1-(5-amino-1-cyclopropyl-8-methoxy-2,4-dioxo-1,2,3,4-tetrahydro-quinazolin-7-yl)-pyrrolidin-3-yl]-2,2-dimethyl-3-methylamino-propionitrile;
3-[1-(3,5-Diamino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile;
3-[1-(5-amino-1-cyclopropyl-8-methyl-2,4-dioxo-1,2,3,4-tetrahydro-quinazolin-7-yl)-pyrrolidin-3-yl]-2,2-dimethyl-3-methylamino-propionitrile;
3-[2-(3-Amino-1-cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile;
3-[2-(3-Amino-1-cyclopropyl-8-methoxy-2,4-dioxo-1,2,3,4-tetrahydro-quinazolin-7-yl)-octahydro-cyclopenta[c]pyrrol-4-ylamino]-propionitrile;
3-[2-(1-Cyclopropyl-6-fluoro-8-methyl-2,4-dioxo-1,2,3,4-tetrahydro-quinazolin-7-yl)-octahydro-cyclopenta[c]pyrrol-4-ylamino]-propionitrile;
3-[2-(1-Cyclopropyl-6-fluoro-8-methoxy-2,4-dioxo-1,2,3,4-tetrahydro-quinazolin-7-yl)-octahydro-cyclopenta[c]pyrrol-4-ylamino]-propionitrile;
3-[2-(1-Cyclopropyl-8-methoxy-2,4-dioxo-1,2,3,4-tetrahydro-quinazolin-7-yl)-octahydro-cyclopenta[c]pyrrol-4-ylamino]-propionitrile; and
3-[2-(1-Cyclopropyl-8-methyl-2,4-dioxo-1,2,3,4-tetrahydro-quinazolin-7-yl)-octahydro-cyclopenta[c]pyrrol-4-ylamino]-propionitrile.

18. A pharmaceutical formulation comprising a compound of formula I, II, III, IV, V, or VI admixed with a pharmaceutically acceptable diluent, carrier, or excipient.

19. A method of treating a bacterial infection in a mammal, comprising administering to a mammal in need thereof an effective amount of a compound of formula I, II, III, IV, V, or VI.
